(19) United States
(12) Patent Application Publication
(10) Pub. No.: US 2025/0248939 A1
(43) Pub. Date: Aug. 7, 2025

(54) LIPID NANOPARTICLES, NUCLEIC ACIDS, AND METHODS OF USE

(71) Applicant: City of Hope, Duarte, CA (US)

(72) Inventors: Marcin Tomasz Kortylewski, Monrovia, CA (US); Elaine Yanan Kang, Duarte, CA (US)

(21) Appl. No.: 18/856,958

(22) PCT Filed: Apr. 18, 2023
(86) PCT No.: PCT/US2023/065880

§ 371 Date: Oct. 15, 2024

Related U.S. Application Data
(60) Provisional application No. 63/332,192, filed on Apr. 18, 2022.

(51) Int. Cl. A61K 9/1272; A61K 9/51; A61P 35/00; B82Y 5/00; C12N 15/113; C12N 15/117

(52) U.S. Cl. CPC A61K 9/1272; A61K 9/5123; A61P 35/00; C12N 15/113; C12N 15/117; B82Y 5/00; C12N 2310/11; C12N 2310/14; C12N 2310/17

(57) ABSTRACT

Provided herein are, inter alia, nucleic acids, lipid nanoparticles, lipid nanoparticles comprising nucleic acids encapsulated therein, pharmaceutical compositions comprising lipid nanoparticles which comprise nucleic acids encapsulated therein, methods of treating diseases, such as cancer, and methods of delivering lipid nanoparticles to myeloid cells, lymphoid organs, and tumors.

### CROSS-REFERENCE TO RELATED APPLICATIONS

This application claims the benefit of priority to U.S. Application No. 63/332,192 filed Apr. 18, 2022, the disclosure of which is incorporated by reference herein in its entirety.

### STATEMENT AS TO RIGHTS TO INVENTIONS MADE UNDER FEDERALLY SPONSORED RESEARCH AND DEVELOPMENT

This invention was made with government support under grant nos. CA107399 and CA213131 awarded by National Institutes of Health. The government has certain rights in the invention.

### REFERENCE TO A SEQUENCE LISTING, A TABLE, OR A COMPUTER PROGRAM LISTING APPENDIX SUBMITTED AS AN ASCII FILE

The Sequence Listing written in file 048440-837001WO_ST26, created 6 Apr. 2023, having 128,121 bytes, machine format IBM-PC, MS Windows operating system, is hereby incorporated by reference.

### BACKGROUND

Lipid nanoparticles (LNPs) are effective drug delivery systems for biologically active compounds, such as therapeutic nucleic acids, proteins, and peptides, which are otherwise cell impermeable. Drugs based on nucleic acids, which include large nucleic acid molecules such as, in vitro transcribed messenger RNA as well as smaller polynucleotides that interact with a messenger RNA or a gene, have to be delivered to the proper cellular compartment in order to be effective. For example, double-stranded nucleic acids such as double-stranded RNA molecules (dsRNA), including siRNAs suffer from their physico-chemical properties that render them impermeable to cells. Upon delivery into the proper compartment, siRNAs block gene expression through a highly conserved regulatory mechanism known as RNA interference (RNAi). Typically, siRNAs are highly anionic due to their phosphate backbone with up to 50 negative charges. In addition, the two complementary RNA strands result in a rigid helix. These features contribute to the siRNA's poor “drug-like” properties. When administered intravenously, the siRNA is rapidly excreted from the body with a typical half-life in the range of only 10 minutes. Additionally, siRNAs are rapidly degraded by nucleases present in blood and other fluids or in tissues and have been shown to stimulate strong immune responses in vitro and in vivo. mRNA molecules suffer from similar issues of impermeability, fragility, and immunogenicity.

Lipid nanoparticle formulations have improved nucleic acid delivery in vivo. For example, such formulations have significantly reduced siRNA doses necessary to achieve target knockdown in vivo. Typically, such lipid nanoparticle drug delivery systems are multi-component formulations comprising cationic lipids, helper lipids, and lipids containing polyethylene glycol. The positively charged cationic lipids bind to the anionic nucleic acid, while the other components support a stable self-assembly of the lipid nanoparticles.

There is a need in the art for improved lipid nanoparticles and for improved delivery of lipid nanoparticles. The disclosure is directed to this, as well as other, important ends.

### BRIEF SUMMARY

Provided herein are lipid nanoparticles comprising a cationic lipid (e.g., a dilinoleic cationic lipid), a phospholipid, a sterol, and a polyethylene glycol-lipid conjugate. In embodiments, the lipid nanoparticles comprise MC3, HSPC, cholesterol, and PEG2000-DMG. In embodiments, the lipid nanoparticles comprise MC3, DPPG, cholesterol, and PEG2000-DMG. In embodiments, the lipid nanoparticles comprise MC3, HSPC, cholesterol, cholesteryl hemisuccinate, and PEG2000-DMG. In embodiments, the lipid nanoparticles encapsulate a nucleic acid, such as RNA, siRNA, or mRNA. In embodiments, the lipid nanoparticles encapsulate a nucleic acid, such as RNA, miRNA, siRNA, or mRNA.

Provided herein are nucleic acids comprising a CpG ODN attached to a sense strand of STAT3 siRNA. In embodiments, the nucleic acids comprise a CpG ODN attached to a sense strand of STAT3 siRNA, wherein the sense strand of STAT3 siRNA is hybridized to a complementary antisense strand of STAT3 siRNA. Provided herein are nucleic acids comprising a sense strand of STAT3 siRNA. In embodiments, the nucleic acids comprise a sense strand of STAT3 siRNA is hybridized to a complementary antisense strand of STAT3 siRNA.

Provided herein are nucleic acids comprising a CpG ODN attached to an antisense strand of STAT3 siRNA. In embodiments, the nucleic acids comprise a CpG ODN attached to an antisense strand of STAT3 siRNA, wherein the antisense strand of STAT3 siRNA is hybridized to a complementary sense strand of STAT3 siRNA. Provided herein are nucleic acids comprising an antisense strand of STAT3 siRNA. In embodiments, the nucleic acids comprise a an antisense strand of STAT3 siRNA is hybridized to a complementary sense strand of STAT3 siRNA.

Provided herein are methods of delivering a lipid nanoparticle to a myeloid cell in a patient comprising administering to the patient lipid nanoparticles or pharmaceutical compositions comprising lipid nanoparticles as described herein. Provided herein are methods of delivering a lipid nanoparticle to a lymphoid organ or a tumor in a patient comprising administering to the patient lipid nanoparticles or pharmaceutical compositions comprising lipid nanoparticles as described herein. Provided herein are methods of treating cancer in a patient comprising administering to the patient lipid nanoparticles or pharmaceutical compositions comprising lipid nanoparticles as described herein. In embodiments, the cancer is lymphoma, leukemia, glioma, head and neck cancer, or prostate cancer.

These and other embodiments of the disclosure are described herein.

### BRIEF DESCRIPTION OF THE DRAWINGS

FIGS. 1A-1I provide characterization of CSI-1A encapsulated LNP. FIGS. 1A-1D: Selected formulated CSI-1A were deposited to cooper grid and counter stained using uranyl acetate, morphology of these LNPs were assessed through transmission electron microscope (TEM) indicating uniformed morphology for LNP-1 & 2 compared to 3 & 4. Each formulated CSI-1A was characterized through Nanosight (FIG. 1E) and encapsulation efficiency (n=5) using Quant-it RNA quantification kit (FIG. 1F) indicating the average size for LNP-1 and LNP-2 were about 80 nm and LNP-3 and LNP-4 were about 120 nm indicating more aggregation/consistency in production. FIG. 1G: Surface charge of formulated CSI-1A was analyzed through zeta potential (Brookaven) with slight negative charge (−5 mV to −20 mV). FIG. 1H: CSI-1A alone, two selected LNP formulations (LNP-1, LNP-2) and solubilized LNP CSI-1A (prepared by incubation at 37° C. with Triton X) were loaded to native PAGE gel and visualized to ensure proper loading and release. FIG. 1I: 20 μM LNP-2 (CSI-1A) were incubated with human serum (1:1 ratio) at 37 degree Celsius for 1-7 days, cargo was extracted and loaded onto native gel and visualized to analyze overall stability of the formulation in human serum.

FIGS. 2A-2G show immune cell-selective uptake and immunostimulatory properties of naked and LNP-encapsulated CpG-STAT3 siRNA (CSI-1A) in vitro. FIGS. 2A-2B: Naked and two selected CSI-1A^{Cy3} formulation were incubated with human PBMCs for 1 hour (top row) and 4 hours (bottom row) in vitro, shown are representative flow histograms showing the uptake to monocyte, mDC, pDC, B cells and T cells. FIG. 2C: Summarized flow cytometric analysis demonstrating the uptake of fluorescently labeled LNPs by human PBMCs from different donors after 4 h of incubation (equivalent 100 nM CSI-1A) (n=3). FIGS. 2D-2E: IFN-α and IL-6 production by hPBMCs after 48 h of exposure to naked or LNP-encapsulated CSI-1A at 500 nM (n=8 and n=4). Selection of formulations with minimal LNP-dependent/CpG-independent immunostimulatory effects. FIGS. 2F-2G: Stimulation of NF-κB-dependent gene activation in RAW-Blue reporter cells treated with different CSI-1A encapsulated in LNPs or with control LNPs encapsulating a non-CpG oligonucleotide control.

FIGS. 3A-3D show the LNP-encapsulated CSI-IA improved target silencing and antitumor efficacy against human B cell lymphoma. FIGS. 3A-3B: More effective STAT3 silencing in human U251 glioma and in human OCI.Ly3 B-cell lymphoma cells after 72 h of treatment using 100 nM concentrations of the indicated oligonucleotides. Shown are results of Western blotting with quantification in relation to β-actin as a loading control. FIG. 3C: enhanced cell killing activity of LNP-2 (CSI-1A) in comparison to human siSTAT3 or mouse siSTAT3 encapsulated LNP-2 at various concentration indicating combinatorial effect of CpG and siSTAT3 in human lymphoma cell killing. FIG. 3D: LNP-2 encapsulation enhanced antitumor efficacy of CSI-1A against OCI.Ly3 lymphoma in immunodeficient NSG mice. Mice were injected s.c. with 1×10⁶ of Ly3 cells. After 15 days, mice were treated using 0.5 mg/kg of CSI-1A, LNP-1 (CSI-1A) and LNP-2 (CSI-1A) every other day for total of six injections. Shown are means±SEM (n=7).

FIGS. 4A-4C show STAT3 silencing in target human cancer cells U251 glioma (FIG. 4A), SCC1 head and neck squamous cell carcinoma (FIG. 4B) and DU145 prostate cancer (FIG. 4C). Note that only sequence #3 tolerated extensive chemical modifications. Cells were transfected with the indicated oligonucleotides for 24 h before changing media. Total cellular lysates were prepared after additional 48 h to detect STAT3 protein levels using Western blotting with beta-actin used as a loading control. Shown are representative results with quantification of STAT3 bands intensities done densitometrically on ChemiDoc Imaging System (Biorad).

FIGS. 5A-5C show that the unformulated but chemically modified CpG-STAT3siRNA (21mer) molecules show improved resistance to human serum nucleases. FIG. 5A: The unformulated (naked) CpG-STAT3siRNA variants #3.3 and #3.4 were incubated at 40 μM in the presence of 50% human serum at 37° C. for the indicated times. Samples were then resolved on native PAGE gels to visualize the amount of intact oligonucleotides at various times. FIGS. 5B-5C: Oligonucleotide band intensities were quantified densitometrically on ChemiDoc Imaging System (Biorad). The results are combined from two independent experiments, means±SD.

FIG. 6 provides a comparison of silencing efficacy of the standard CpG-STAT3siRNA (CSI-1A) and variants thereof encapsulated in the LNP-2 formulation. STAT3 protein levels in target human cancer cells U251 glioma (upper panel) and OCI.Ly3 B cell lymphoma (lower panel) after treatment using 200 nM of indicated oligonucleotides formulated in LNPs (LNP-2). Total cellular lysates were prepared after 72 h treatment to detect STAT3 protein levels using Western blotting with beta-actin used as a loading control. Shown are representative results with quantification of STAT3 bands intensities done densitometrically on ChemiDoc Imaging System (Biorad).

FIGS. 7A-7F show dramatically improved direct antitumor effects of LNP2-encapsulated CpG-STAT3siRNA (CSI-1A) in vitro and in vivo against xenotransplanted human B cell lymphoma. FIGS. 7A-7D: Dose-dependent STAT3 silencing in OCI.Ly3 (FIGS. 7A-7B) and U2946 (FIGS. 7C-7D) cells treated with LNP2-encapsulated CpG-siSTAT3. Direct cytotoxicity of LNP2-encapsulated CpG-STAT3siRNA or CpG alone, STAT3siRNA alone or scramble RNA control on the same lymphoma cells. FIGS. 7E-7F: LNP2 encapsulation enhances potency of CpG-STAT3siRNA-mediated growth inhibitory effect against subcutaneously-engrafted human B cell lymphoma models, Ly3 (n=7) (FIG. 7E) and U2946 (n=5) (FIG. 7F). NSG mice were implanted with 5×10⁶ lymphoma cells and treated using 0.5 mg/kg of oligonucleotide peritumorally at 15 days or 26 days after tumor engraftment; shown are means±SEM. Each experiment was performed twice.

FIGS. 8A-8F show cell-selective uptake of different LNP (CpG-siRNA) formulations by healthy human PBMC. FIG. 8A: details of tested LNP compositions including ratios of various components. FIG. 8B: physiochemical characterization of three LNP (CpG-siRNA) formulations (DLS, size; Zeta, surface charge). FIGS. 8C-8D: cell-selective uptake of three LNP variants (100 nM) loaded with fluorescently-labeled cargo (CpG-siRNA^{Cy3}), shown are representative histograms of uptake by primary human PBMCs (4 h) and graphs summarizing mean fluorescent intensity (MFI) for various immune cell subsets (monocytes: CD14+, pDC: CD303a+, mDC: CD1c+, B cells: CD19+, T cells: CD3+). FIGS. 8E-8F: results verification using the three LNP (CpG-siRNA) variants (100 nM) labeled with a lipophilic dye (DiD). Shown are means±SEM (n=3).

FIGS. 9A-9F: in vivo biodistribution of systemically administered new LNP-2(CpG-STAT3siRNA) (CSI-1A) formulations in CMM-bearing mice. Leukemia (CMM) bearing mice were administered with about 20 μg of fluorescently DiD-labeled LNP-2(CpG-STAT3siRNA) and major organs were harvested 3 h after retroorbital/intravenous administration (n=3). Shown are bioluminescent images adjusted for the same signal range for organs such as: liver (FIG. 9A), spleen (FIG. 9B), lung (FIG. 9C), kidney (FIG. 9D), and the peripheral lymph nodes (FIG. 9E). FIG. 9F: an organ level uptake of tested formulations. Summarized organ level fluorescent intensities were shown at the lower right corner (relative to control).

FIGS. 10A-10C: the cell-selective internalization of LNP-2(CpG-STAT3siRNA) variants in vivo. AML-bearing mice were injected IV using about 20 μg of LNPDiD and major organs were harvested 3 h later. Immune cell selective uptake in spleen (FIG. 10A), bone morrow (FIG. 10B), and lymph nodes (FIG. 10C). The LNP-2(CpG-STAT3siRNA) uptake by specific mouse immune cell populations was analyzed through flow cytometry (AML cells: eGFP+; B cells: CD19+; T cells: CD3+; monocytes/macrophages: CD11b+/CD11c−; pDC/cDC1: CD11b−/CD11c+; acDC2: CD11b+/CD11c+. Shown are means±SEM (n=3).

FIGS. 11A-11B show results of LNP characterization. FIG. 11A: nanotracking analysis (NTA) was performed of LNP variants using Nanosight; shown are representative average LNP size distributions in three individual measurements. FIG. 11B: LNP2 formulated with miR146a were examined using gel electrophoresis on 15% PAGE comparing a reference miR146a oligonucleotide, LNP2(miR146a) formulation and miR146a extracted from LNPs.

FIGS. 12A-12B: show in vitro activity and anti-inflammatory effect of LNP2(miR146a). FIG. 12A: RAW 264.7 macrophages were incubated with LNP2(miR146a) or transfected using miR146a using Oligofectamine™ at the same concentration (200 nM) for indicated times, and target proteins were analyzed using Western blotting; U.T. —untreated. FIG. 12B: RAW-Blue™ cells were treated using various concentrations of LNP2(miR146a) for 24 or 48 h, and then stimulated with LPS (100 ng/mL) for 4 h. The supernatants were later collected for colorimetric assessment of the NF-κB-driven SEAP activity. Shown are representative results for experiments repeated twice in triplicates, mean±SD.

FIGS. 13A-13C: show cell-selective internalization and activity of LNP2(miR146a) on primary mouse splenocytes. FIGS. 13A-13B: Wild-type C57BL/6 mice splenocytes were incubated with various concentrations of fluorescently-labeled LNP2(miR146a^{Cy3}) for 8 hours and then uptake by different immune cell subsets was assessed using flow cytometry. MACs, macrophages. DCs, dendritic cells. Untreated, U.T. FIG. 13C: Splenocytes from miR146aKO mice were pre-incubated overnight with LNP2(miR146a) or control LNP2 (scrRNA). Cells were then stimulated using LPS (100 ng/mL) for indicated hours and supernatants were collected for ELISA analysis of mouse IL-6 secreted post stimulation. ELISA was performed according to manufacturer's protocol; mean±standard deviation (n=3). **P<0.01; ns, not significant. Two-way ANOVA with multiple comparison of each individual time points.

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

The abbreviations used herein have their conventional meaning within the chemical and biological arts. The chemical structures and formulae set forth herein are constructed according to the standard rules of chemical valency known in the chemical arts.

“CSI-1A” or “CAS3/SS3” refers to the nucleic acid in which SEQ ID NO: 14 is hybridized to SEQ ID NO:8. In particular, the antisense strand of STAT3 siRNA in SEQ ID NO: 14 is hybridized to the complementary sense strand siRNA of SEQ ID NO:8.

“Seq. #3.3” or “#3.3” refers to the nucleic acid in which SEQ ID NO: 15 is hybridized to SEQ ID NO:10. In particular, the sense strand of STAT3 siRNA in SEQ ID NO:15 is hybridized to the complementary antisense strand siRNA of SEQ ID NO: 10.

“Seq. #3.4” or “#3.4” refers to the nucleic acid in which SEQ ID NO:6 is hybridized to SEQ ID NO:12. In particular, the sense strand of STAT3 siRNA in SEQ ID NO:6 is hybridized to the complementary antisense strand siRNA of SEQ ID NO: 12.

The term “lipid” refers to a group of organic compounds that include, but are not limited to, esters of fatty acids and are characterized by being insoluble in water, but soluble in many organic solvents.

The terms “lipid nanoparticle” refers to a lipid formulation that can be used to deliver an active agent or therapeutic agent, such as a nucleic acid (e.g., DNA, RNA, sense RNA, antisense RNA, siRNA, mRNA), to a target site of interest (e.g., tumor, cell, tissue, organ, and the like). In embodiments, the lipid nanoparticle is typically formed from a cationic lipid, a non-cationic lipid, and a conjugated lipid that prevents aggregation of the particle. In other embodiments, the active agent or therapeutic agent, such as a nucleic acid (e.g., DNA, RNA, sense RNA, antisense RNA, siRNA, mRNA), may be encapsulated in the lipid portion of the particle, thereby protecting it from enzymatic degradation.

“Lipid encapsulated nanoparticle” refers to a lipid nanoparticle that provides an active agent or therapeutic agent, such as a nucleic acid (e.g., DNA, RNA, sense RNA, antisense RNA, siRNA, mRNA), with full encapsulation, partial encapsulation, or both. In embodiments, the nucleic acid (e.g., DNA, RNA, sense RNA, antisense RNA, siRNA, mRNA) is fully encapsulated in the lipid particle.

The term “lipid conjugate” refers to a conjugated lipid that inhibits aggregation of lipid particles. Such lipid conjugates include PEG-lipid conjugates such as, e.g., PEG coupled to dialkyloxypropyls (e.g., DAA-PEG conjugates), PEG coupled to diacylglycerols (e.g., DAG-PEG conjugates), PEG coupled to cholesterol, PEG coupled to phosphatidylethanolamines, PEG conjugated to ceramides, cationic PEG lipids, polyoxazoline (POZ)-lipid conjugates (e.g., POZ-DAA conjugates), polyamide oligomers (e.g., ATTA-lipid conjugates), and mixtures thereof. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In embodiments, non-ester containing linker moieties, such as amides or carbamates, are used.

In embodiments, the term “polyethylene glycol-lipid conjugate” or “PEG-lipid conjugate” refers to a polyethylene glycol (PEG) having a molecular weight from about 500 Daltons to about 10,000 Daltons conjugated to a C₁₂-C₂₂ fatty acid lipid. In embodiments, a PEG-lipid conjugate is polyethylene glycol having a molecular weight from about 1,000 Daltons to about 6,000 Daltons conjugated to a C₁₂-C₂₂ fatty acid lipid. In embodiments, a PEG-lipid conjugate is polyethylene glycol having a molecular weight from about 2,000 Daltons to about 5,000 Daltons conjugated to a C₁₂-C₂₀ fatty acid lipid. In embodiments, a PEG-lipid conjugate is polyethylene glycol having a molecular weight from about 2,000 Daltons to about 5,000 Daltons conjugated to a C₁₂-C₁₈ fatty acid lipid. In embodiments, a PEG-lipid conjugate is polyethylene glycol having a molecular weight from about 1,500 Daltons to about 2,500 Daltons conjugated to a C₁₂-C₁₈ fatty acid lipid. In embodiments, a PEG-lipid conjugate is polyethylene glycol having a molecular weight from about 2,000 Daltons conjugated to a C₁₂-C₁₈ fatty acid lipid. In embodiments, the PEG-lipid conjugate is N-palmitoyl-sphingosine-1-{succinyl [methoxy-(polyethylene glycol)]} (C16 PEG ceramide), 1,2-dimyristoyl-rac-glycero-3-methoxy-polyethylene glycol (DMG-PEG), 1,2-dipalmitoyl-sn-glycero-3-phosphoethanolamine-N-[azido(polyethylene glycol) (DPPE-PEG), 1,2-dipalmitoyl-rac-glycero-3-methylpolyoxy-ethylene (DPG-PEG), distearoyl-rac-glycerol (polyethylene glycol) (DSG-PEG), 1,2-distearoyl-sn-glycero-3-phosphoethanolamine-N-[amino(polyethylene glycol) (DSPE-PEG). In embodiments, the polyethylene glycol has an average molecular weight of about 2000 daltons (e.g., DMG-PEG2000, DPPE-PEG2000, DPG-PEG2000, DSG-PEG2000, DSPE-PEG2000). In embodiments, DMG-PEG is a mixture of 1,2-DMG PEG2000 and 1,3-DMG PEG2000. In embodiments, DMG-PEG is a mixture of 1,2-DMG PEG2000 and 1,3-DMG PEG2000 in a ratio of about 99:1 to about 90:10. The polyethylene glycol-lipid conjugate can optionally be in the form of a pharmaceutically acceptable salt (e.g., ammonium salt).

The term “average molecular weight” refers to the average molecular weight of a polymer sample that is determined by a technique known in the art, such as gel permeation chromatography, light-scattering measurements and viscosity measurements. In embodiments, the average molecular weight is the number average molecular weight which is defined as the total weight of polymer divided by the total number of molecules.

The term “amphipathic lipid” refers, in part, to any material wherein the hydrophobic portion of the lipid orients into a hydrophobic phase, while the hydrophilic portion orients toward the aqueous phase. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include long-chain saturated and unsaturated aliphatic hydrocarbon groups and such groups substituted by one or more aromatic, cycloaliphatic, or heterocyclic group(s). Examples of amphipathic compounds include phospholipids, aminolipids, and sphingolipids. Other compounds lacking in phosphorus, such as sphingolipid, glycosphingolipid families, diacylglycerols, and β-acyloxyacids, are also within the group designated as amphipathic lipids. Additionally, the amphipathic lipids can be mixed with other lipids including triglycerides and sterols.

“Phospholipids” are a class of lipids whose molecule has a hydrophilic “head” containing a phosphate group and two hydrophobic “tails” derived from fatty acids, joined by an alcohol residue. Exemplary phospholipids include dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine (MMPE), dimethyl-phosphatidylethanolamine (DMPE), dielaidoyl-phosphatidylethanolamine (DEPE), stearoyloleoyl-phosphatidyl-ethanolamine (SOPE), egg phosphatidylcholine (EPC), hydrogenated soy phosphatidylcholine (HSPC), dipalmitoyl-phosphatidylglycerol (DPPG), and mixtures thereof. In embodiments, DSPC is 1,2-distearoyl-sn-glycero-3-phosphocholine. In embodiments, DPPG is 1,2-dipalmitoyl-phosphatidyl-glycerol. In embodiments, DPPG is 1,2-dihexadecanoyl-sn-glycero-3-phospho-(1′-sn-glycerol).

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “hydrophobic lipid” refers to compounds having apolar groups that include long-chain saturated and unsaturated aliphatic hydrocarbon groups and such groups optionally substituted by one or more aromatic, cycloaliphatic, or heterocyclic group(s). Examples include diacylglycerol, dialkylglycerol, N—N-dialkylamino, 1,2-diacyloxy-3-aminopropane, and 1,2-dialkyl-3-aminopropane.

A “cationic lipid” is a positively charged lipid that has the ability to form aggregate complexes with anionic nucleic acids (such as DNA or RNA). Exemplary cationic lipids include N,N-dioleyl-N,N-dimethylammonium chloride (DODAC), 1,2-dioleyloxy-N,N-dimethyl-aminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), 1,2-dioleoyloxy-3-(trimethylammonium)propane (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamido)ethyl]-N,N-dimethyl-1-propanaminium-trifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)-propane (CLinDMA), 2-[5′-(cholest-5-en-3-beta-oxy)-3′-oxapentoxy)-3-dimethy-1-(cis,cis-9′,1-2′-octadecadienoxy)-propane (CpLinDMA), N,N-dimethyl-3,4-dioleyloxybenzylamine (DMOBA), 1,2-N,N′-dioleylcarbamyl-3-dimethyl-aminopropane (DOcarbDAP), 3-(N,N-dioleylamino)-1,2-propanedio (DOAP), and 1,1′-((2-(4-(2-((2-(bis(2-hydroxydodecyl)amino)ethyl) (C12-200). In embodiments, the cationic lipid is a “dilinoleic cationic lipid” as defined herein. In embodiments, the term “cationic lipid” does not include a “dilinoleic cationic lipid.”

As used herein, the term “dilinoleic cationic lipid” refers to any cationic lipid containing two linoleic moieties (e.g., two C₁₈ moieties optionally containing 1, 2, or 3 —CH═CH— groups). In embodiments, a dilinoleic cationic moiety comprises two —(CH₂)₈CH═CHCH₂CH═CH(CH₂)₄CH₃ moieties. Exemplary dilinoleic cationic lipids include MC3, MC3 derivatives, 1,2-dilinoleyloxy-N,N-dimethylaminopropane (DLinDMA), 2,2-dilinoleyl-4-(2-dimethylaminoethyl)-[1,3]-dioxolane (DLin-KC2-DMA), 2,2-dilinoleyl-4-(3-dimethylaminopropyl)-[1,3]-dioxolane (DLin-K-3-DMA), 2,2-dilinoleyl-4-(4-dimethylaminobutyl)-[1,3]-dioxolane (DLin-KC4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane or a salt thereof (DLin-TMA), 1,2-dilinoleoyl-3-trimethylaminopropane or a salt thereof (DLin-TAP), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino) ethoxypropane (DLin-EG-DMA), and 1,2-N,N′-dilinoleylcarbamyl-3-dimethylaminopropane (DLincarbDAP).

The term “MC3” or “Dlin-MC3-DMA” refer to dilinoleyl-methyl-4-dimethylaminobutyrate. In embodiments, “MC3” refers to heptatriaconta-6,9,28,31-tetraen-19-yl 4-(dimethylamino) butanoate. In embodiments, “MC3” refers to (6Z,9Z,28Z,31Z)-heptatriaconta-6,9,28,31-tetraen-19-yl-4-(dimethylamino) butanoate.

The term “MC3 derivative” refers to derivatives of MC3 such as those described in US Publication No. 2017/0151333. Exemplary MC3 derivatives include LenMC3, γ-LenMC3, MC3 ether, MC4 ether, MC3MC, MC2C, MC2MC, MC3 thioester, MC3 alkyne, MC3 amide, and other compounds described in US Publication No. 2017/0151333.

The term “non-lamellar morphology” refer to a non-bilayer structure. The non-bilayer morphology can include, for example, three dimensional tubes, rods, cubic symmetries, etc. The non-lamellar morphology (i.e., non-bilayer structure) of the lipid particles can be determined using analytical techniques including Cryo-Transmission Electron Microscopy (“Cryo-TEM”), Differential Scanning calorimetry (“DSC”), and X-Ray Diffraction.

The term “a plurality of nucleic acid-lipid particles” refers to at least 2 particles, more preferably more than 10², 10³, 10⁴, 10⁵, 10⁶ or more particles (or any fraction thereof or range therein). In embodiments, the plurality of nucleic acid-lipid particles includes 50-100, 50-200, 50-300, 50-400, 50-500, 50-600, 50-700, 50-800, 50-900, 50-1000, 50-1100, 50-1200, 50-1300, 50-1400, 50-1500, 50-1600, 50-1700, 50-1800, 50-1900, 50-2000, 50-2500, 50-3000, 50-3500, 50-4000, 50-4500, 50-5000, 50-5500, 50-6000, 50-6500, 50-7000, 50-7500, 50-8000, 50-8500, 50-9000, 50-9500, 50-10,000 or more particles.

The term “organic lipid solution” refers to a composition comprising in whole, or in part, an organic solvent having a lipid.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of nucleic acids contemplated herein include single and double stranded DNA, single and double stranded RNA, and hybrid molecules having mixtures of single and double stranded DNA and RNA. Examples of nucleic acids contemplated herein include any types of RNA (e.g., antisense RNA, mRNA, siRNA, miRNA, shRNA, guide RNA, dicer substrate RNA, dicer substrate siRNAs (dsiRNAs) (dsiRNA are cleaved by the RNase III class endoribonuclease dicer into 21-23 base duplexes having 2-base 3′-overhangs siRNA), and any type of DNA, genomic DNA, plasmid DNA, minicircle DNA, minigene, and any fragments thereof. The term “duplex” in the context of nucleic acids refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as 2′O-methyl, 2′O-methoxyethoxy, 2′fluoro, 5-methyl cytidine or pseudouridine; and peptide nucleic acid backbones and linkages. Other analog nucleic acids include those with positive backbones; non-ionic backbones, modified sugars (e.g., deoxyribose), and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A “CpG ODN” or CpG oligodeoxynucleotide” refers to a synthetic single-stranded DNA containing CpG motifs. In embodiments, the CpG ODN is a Class A CpG ODN, a Class B CpG ODN, or a Class C CpG ODN.

The term “Class A CpG ODN” refers to a CpG motif including oligodeoxynucleotide including one or more of poly-G sequence at the 5′, 3′, or both ends; an internal palindrome sequence including CpG motif; or one or more phosphodiester derivatives linking deoxynucleotides. In embodiments, a Class A CpG ODN includes poly-G sequence at the 5′, 3′, or both ends; an internal palindrome sequence including CpG motif; and one or more phosphodiester derivatives linking deoxynucleotides. In embodiments, the phosphodiester derivative is phosphorothioate Examples of Class A CpG ODNs include ODN D19, ODN 1585, ODN 2216, and ODN 2336, the sequences of which are known in the art.

The term “Class B CpG ODN” refers to a CpG motif including oligodeoxynucleotide including one or more of a 6mer motif including a CpG motif; phosphodiester derivatives linking all deoxynucleotides. In embodiments, a Class B CpG ODN includes one or more copies of a 6mer motif including a CpG motif and phosphodiester derivatives linking all deoxynucleotides. In embodiments, the phosphodiester derivative is phosphorothioate. In embodiments, a Class B CpG ODN includes one 6mer motif including a CpG motif. In embodiments, a Class B CpG ODN includes two copies of a 6mer motif including a CpG motif. In embodiments, a Class B CpG ODN includes three copies of a 6mer motif including a CpG motif. In embodiments, a Class B CpG ODN includes four copies of a 6mer motif including a CpG motif. Examples of Class B CpG ODNs include ODN 1668, ODN 1826, ODN 2006, ODN 2007, ODN BW006, and ODN D-SL01, the sequences of which are known in the art.

The term “Class C CpG ODN” refers to an oligodeoxynucleotide including a palindrome sequence including a CpG motif and phosphodiester derivatives (phosphorothioate) linking all deoxynucleotides. Examples of Class C CpG ODNs include ODN 2395, ODN M362, and ODN D-SL03, the sequences of which are known in the art.

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and anomeric sugar-phosphate, backbone-modified nucleotides.

In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include small interfering RNAs (siRNAs) (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

“Hybridize” and “hybridization” refer to the pairing of complementary (including partially complementary) nucleic acid strands. Hybridization and the strength of hybridization (e.g., the strength of the association between nucleic acid strands) is impacted by factors known in the art including the degree of complementarity between the nucleic acid, stringency of the conditions involved affected by such conditions as the concentration of salts, the melting temperature (Tm) of the formed hybrid, the presence of other components, the molarity of the hybridizing strands and the G: C content of the nucleic acid strands. When one nucleic acid is said to “hybridize” to another nucleic acid, it means that there is some complementarity between the two nucleic acids or that the two nucleic acids form a hybrid under high or low stringency conditions.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The phrase “hybridization conditions” refers to conditions under which a nucleic acid will hybridize to its target sequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary hybridization conditions can be as follows: 50% formamide, 5×SSC, and 1% SDS, incubating at 42° C., or 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For PCR amplification, a temperature of about 62° C. is typical, although high stringency annealing temperatures can range from about 50° C. to about 65° C. depending on the primer length and specificity. Typical cycle conditions for both high and low stringency amplifications include a denaturation phase of 90° C.-95° C. for 30 seconds to 2 minutes, an annealing phase lasting 30 seconds to 2 minutes, and an extension phase of about 72° C. for 1-2 min. Protocols and guidelines for low and high stringency amplification reactions are provided, e.g., in Innis et al., PCR Protocols, A Guide to Methods and Applications, Academic Press, Inc. N. Y. (1990).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (e.g., http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In embodiments, the nucleic acids described herein are isolated nucleic acids.

The term “activation,” “activate,” “activating,” “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The term “inhibition,” “inhibit,” “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “pharmaceutically acceptable salts” refers to salts of the active compounds that are prepared with relatively nontoxic acids or bases, depending on the particular substituents found on the compounds described herein. Any of the compounds described herein can be in the form of a pharmaceutically acceptable salt. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The term “heteroalkyl,” by itself or in combination with another term, means, unless otherwise stated, a stable non-cyclic straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH—CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃.

The term “heteroalkylene,” by itself or as part of another substituent, means, unless otherwise stated, a divalent radical derived from heteroalkyl, as exemplified, but not limited by, —CH₂—CH₂—S—CH₂—CH₂—, —O—CH₂—CH₂—NH—CH₂—, —O—(CH₂)₃—O—PO₃—, —O—(CH₂)—O—PO₃—, —O—(CH₂)₂—O—PO₃—, —O—(CH₂)₄—O—PO₃—, and the like. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O) NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

A “substituent group,” as used herein, means a group selected from the following moieties: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl). In embodiments, a “substituent group” is alkyl (e.g., C₁-C₈alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: (i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (ii) alkyl (e.g., C₁-C₈alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: (a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

The terms “treating” or “treatment” refers to any indicia of success in the therapy or amelioration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination. The term “treating” and conjugations thereof, may include prevention of a pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

“Patient” or “subject” refers to a living organism. Non-limiting examples include humans, other mammals, dogs, cats, bovines, rats, mice, dogs, monkeys, goat, sheep, cows, and other non-mammalian animals. In embodiments, a patient is a cat or a dog. In embodiments, a patient is a mammal. In embodiments, a patient is a primate. In embodiments, a patient is human.

A “effective amount” as used herein, is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). In these methods, the effective amount of the nucleic acid (DNA, RNA, antisense RNA, siRNA, mRNA) described herein is an amount effective to accomplish the stated purpose of the method. An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically effective amount,” as used herein, refers to that amount of therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. For any compound described herein, therapeutically effective amount can be initially determined from cell culture assays. Target concentrations will be those concentrations of active compound(s) that are capable of achieving the methods described herein, as measured using the methods described herein or known in the art. As is in the art, therapeutically effective amounts for use in humans can also be determined from animal models. For example, a dose for humans can be formulated to achieve a concentration that has been found to be effective in animals.

For any compound described herein, the therapeutically effective amount can be initially determined from cell culture assays. Target concentrations will be those concentrations of active compound that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “administering” means intranasal administration, inhalation administration, oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. In embodiments, administering does not include administration of any active agent other than the nucleic acid. In embodiments, administration is intranasal. In embodiments, administration is intravenous. In embodiments, administration is intranasal administration of lipid nanoparticles. In embodiments, administration is intravenous administration of lipid nanoparticles.

The singular terms “a,” “an,” and “the” include the plural reference unless the context clearly indicates otherwise.

### Nucleic Acids

Provided herein are nucleic acids. In embodiments, the nucleic acids are RNA or DNA. In embodiments, the nucleic acids are RNA. In embodiments, the nucleic acids are mRNA. In embodiments, the nucleic acids are miRNA. In embodiments, the nucleic acids are siRNA. In embodiments, the nucleic acids are STAT3 siRNA. In embodiments, the nucleic acids are DNA. In embodiments, the nucleic acids are CpG oligodeoxynucleotides (ODN). In embodiments, the nucleic acids are CpG ODN linked to miRNA. In embodiments, the nucleic acids are CpG ODN linked to siRNA. In embodiments, the nucleic acids are CpG ODN linked to STAT3 siRNA. In embodiments, the nucleic acids are CpG ODN linked to the antisense strand of STAT3 siRNA. In embodiments, the nucleic acids are CpG ODN linked to the antisense strand of STAT3 siRNA, wherein the antisense strand of the STAT3 siRNA is hybridized to the complementary sense strand of STAT3 siRNA (i.e., the antisense strand of the STAT3 siRNA is hybridized to the sense strand of STAT3 siRNA). In embodiments, the nucleic acids are CpG ODN linked to the sense strand of STAT3 siRNA. In embodiments, the nucleic acids are CpG ODN linked to the sense strand of STAT3 siRNA, wherein the sense strand of the STAT3 siRNA is hybridized to the complementary antisense strand of STAT3 siRNA (i.e., the sense strand of the STAT3 siRNA is hybridized to the antisense strand of STAT3 siRNA).

In embodiments, CpG ODN are linked to RNA (e.g., siRNA, miRNA) via a bond, a chemical moiety, nucleic acids, or a combination thereof. In embodiments, the 3′ end of the CpG ODN is linked to the 5′ end of the RNA (e.g., siRNA, miRNA) via a bond, a chemical moiety, nucleic acids, or a combination thereof. In embodiments, the linking group is a substituted or unsubstituted heteroalkylene. In embodiments, the linking group is a substituted heteroalkylene. In embodiments, the 3′ end of the CpG ODN is linked to the 5′ end of STAT3 siRNA via an unsubstituted or substituted heteroalkylene. In embodiments, the 3′ end of the CpG ODN is linked to the 5′ end of STAT3 siRNA via a substituted heteroalkylene. In embodiments, the heteroalkylene is a substituted 18 to 42 membered heteroalkylene. In embodiments, the heteroalkylene is a substituted 24 to 36 membered heteroalkylene. In embodiments, the heteroalkylene comprises an oxygen atom, a phosphorous atom, or a combination thereof. In embodiments, the substituents on the substituted heteroalkylene comprise oxo, —OH, or a combination thereof. In embodiments, the substituted heteroalkylene is:

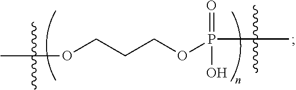

wherein n is an integer from 1 to 10. In embodiments, n is an integer from 4 to 6. In embodiments, n is 5.

Provided herein are nucleic acids having the sequences shown in Table 1.

TABLE 1

| SEQ | |
|---|---|
| ID NO: | Sequences (5′→3′) |
| 1 | UCA GUC GUA UCU UUC UGC AGC UUC CGU |
| 2 | GGA AGC UGC AGA AAG AUA CGA CUG A |
| 3 | AGG AUG ACU UUG AUU UCA ACU |
| 4 | AGU UGA AAU CAA AGU CAU CCU UU |
| 5 | GGT GCA TCG ATG CAG GGG GG |
| 16 | UGAGAAC:UGAAUUCCAUGGGUU |
| 17 | CCCAUGGAAUUCAGUUCUCAAA |

In embodiments, the nucleic acids in Table 1 are DNA or RNA. In embodiments, SEQ ID NOS: 1-4 are RNA. In embodiments, SEQ ID NOS: 1-4 are siRNA. In embodiments, SEQ ID NO: 5 is DNA. In embodiments, SEQ ID NO:16 and SEQ ID NO: 17 are miRNA. In embodiments, SEQ ID NO:5 is bonded to SEQ ID NO:1. In embodiments, SEQ ID NO:5 is bonded to SEQ ID NO:2. In embodiments, SEQ ID NO:5 is bonded to SEQ ID NO: 1, and SEQ ID NO: 1 is hybridized to SEQ ID NO:2. In embodiments, SEQ ID NO:5 is bonded to SEQ ID NO: 3. In embodiments, SEQ ID NO:5 is bonded to SEQ ID NO:3, and SEQ ID NO:3 is hybridized to SEQ ID NO:4. In embodiments, SEQ ID NO:5 is bonded to SEQ ID NO:4. In embodiments, SEQ ID NO: 16 is hybridized to SEQ ID NO:17.

In embodiments, a nucleotide in any one of SEQ ID NOS: 1-5 is modified. In embodiments, SEQ ID NO: 16 and/or SEQ ID NO: 17 is modified. In embodiments, the nucleic acid comprises a modified base, a modified sugar, a modified phosphate, or a combination of two or more thereof. In embodiments, the nucleic acid comprises a modified sugar and a modified phosphate. In embodiments, the nucleic acid comprises a modified base and a modified phosphate. In embodiments, the nucleic acid comprises a modified base and a modified sugar. In embodiments, the nucleic acid comprises a modified base. In embodiments, the nucleic acid comprises a modified sugar. In embodiments, the nucleic acid comprises a modified phosphate. In embodiments, the modified base is 2′O-Methyl modified base, a 2′O-methoxyethoxy modified base, a 2′fluoro modified base, a 5-methyl-modified cytidine, or pseudouridine. In embodiments, the modified base is a 2′O-Methyl modified base. In embodiments, the modified phosphate is phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite. In embodiments, the modified phosphate is phosphorothioate. In embodiments, the modified sugar is deoxyribose. In embodiments, a nucleotide in any one of SEQ ID NOS: 1-5 is modified with 2′-O-methyl, 2′fluoro, phosphorothioate, or a combination thereof. In embodiments, a nucleotide in any one of SEQ ID NOS: 1-5 is modified with 2′-O-methyl, 2′fluoro, phosphorothioate, or a combination thereof. In embodiments, a nucleotide in any one of SEQ ID NOS: 1-4 is modified with 2′-O-methyl. In embodiments, a nucleotide in any one of SEQ ID NOS: 1-4 is modified with 2′fluoro. In embodiments, a nucleotide in any one of SEQ ID NOS: 1-4 is modified with 2′-O-methyl and phosphorothioate. In embodiments, a nucleotide in any one of SEQ ID NOS: 1-4 is modified with 2′fluoro and phosphorothioate. In embodiments, a nucleotide in any one of SEQ ID NO:5 is modified with phosphorothioate.

Provided herein are nucleic acids having the sequences shown in Table 2. In embodiments, the nucleic acid comprises SEQ ID NO:7. In embodiments, the nucleic acid comprises SEQ ID NO:8. In embodiments, the nucleic acid comprises SEQ ID NO:9. In embodiments, the nucleic acid comprises SEQ ID NO: 10. In embodiments, the nucleic acid comprises SEQ ID NO: 11. In embodiments, the nucleic acid comprises SEQ ID NO: 12. In embodiments, the nucleic acid comprises SEQ ID NO: 13. In embodiments, the nucleic acid comprises SEQ ID NO:7 hybridized to the complementary nucleic acid comprising SEQ ID NO: 8. In embodiments, the nucleic acid comprises SEQ ID NO:9 hybridized to the complementary nucleic acid comprising SEQ ID NO: 10. In embodiments, the nucleic acid comprises SEQ ID NO: 11 hybridized to the complementary nucleic acid comprising SEQ ID NO: 12.

TABLE 2

| SEQ | |
|---|---|
| ID NO: | Sequences (5′→3′) |
| 7 | UCA GUC GUA UCU UUC UGC AGC UUC CmGU |
| 8 | GGA AGC UGC AGA AAG AUA CGA CUdG dA |
| 9 | mA*mG*G mAUmG ACU UUmG AmUU mUCmA ACU |
| 10 | A*G*U UmGA mAAmU CAA AGU CAU mCCmU* |
| | mU*mU |
| 11 | mA*mG*mG mAmUmG fACfU fUfUmG AmUmU |
| | mUmCmA mAmCmU |
| 12 | mA*fG*mU mUmGfA mAmAmU mCmAmA mAfGmU |
| | fCmAmU mCmCmU* mU*mU |
| 13 | G*G*T GCA TCG ATG CAG G*G*G* G*G |

Provided herein is a nucleic acid comprising a CpG ODN and SEQ ID NO:7. In embodiments, the CpG ODN is any CpG ODN known in the art. In embodiments, the CpG ODN is Class A CpG ODN (e.g., ODN D19, ODN 1585, ODN 2216, ODN 2336), a Class B CpG ODN (e.g., ODN 1668, ODN 1826, ODN 2006, ODN 2007, ODN BW006, ODN D-SL01), or a Class C CpG ODN (e.g., ODN 2395, ODN M362, ODN D-SL03). In embodiments, the nucleic acid comprises SEQ ID NO:13 and SEQ ID NO:7. In embodiments, SEQ ID NO:13 is bonded to SEQ ID NO:7. In embodiments, SEQ ID NO: 13 is bonded via a linking group to SEQ ID NO: 7. In embodiments, the linking group is a bond, a nucleic acid, or a chemical moiety. In embodiments, SEQ ID NO: 13 is covalently bonded via a substituted or unsubstituted heteroalkylene to SEQ ID NO:7. In embodiments, SEQ ID NO:13 is covalently bonded via a substituted or unsubstituted heteroalkylene to SEQ ID NO:7, and SEQ ID NO:7 is hybridized to the complementary SEQ ID NO:8. In embodiments, SEQ ID NO:7 is an antisense strand of STAT3 siRNA and SEQ ID NO:8 is the complementary sense strand of STAT3 siRNA. In embodiments, the heteroalkylene is a substituted 18 to 42 membered heteroalkylene. In embodiments, the heteroalkylene is a substituted 24 to 36 membered heteroalkylene. In embodiments, the heteroalkylene comprises an oxygen atom, a phosphorous atom, or a combination thereof. In embodiments, the substituents on the substituted heteroalkylene comprise oxo, —OH, or a combination thereof. In embodiments, the substituted heteroalkylene is:

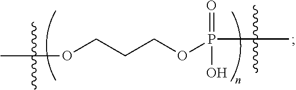

wherein n is an integer from 1 to 10. In embodiments, n is an integer from 4 to 6. In embodiments, n is 5.

Provided herein is a nucleic acid comprising a CpG ODN and SEQ ID NO:9. In embodiments, the CpG ODN is any CpG ODN known in the art. In embodiments, the CpG ODN is Class A CpG ODN (e.g., ODN D19, ODN 1585, ODN 2216, ODN 2336), a Class B CpG ODN (e.g., ODN 1668, ODN 1826, ODN 2006, ODN 2007, ODN BW006, ODN D-SL01), or a Class C CpG ODN (e.g., ODN 2395, ODN M362, ODN D-SL03). In embodiments, the nucleic acid comprises SEQ ID NO:13 and SEQ ID NO:9. In embodiments, SEQ ID NO: 13 is bonded to SEQ ID NO:9. In embodiments, SEQ ID NO:13 is bonded via a linking group to SEQ ID NO: 9. In embodiments, the linking group is a bond, a nucleic acid, or a chemical moiety. In embodiments, SEQ ID NO:13 is covalently bonded via a substituted or unsubstituted heteroalkylene to SEQ ID NO:9. In embodiments, SEQ ID NO:13 is covalently bonded via a substituted or unsubstituted heteroalkylene to SEQ ID NO:9, and SEQ ID NO:9 is hybridized to SEQ ID NO:10. In embodiments, SEQ ID NO:9 is a sense strand of STAT3 siRNA and SEQ ID NO: 8 is the complementary antisense strand of STAT3 siRNA. In embodiments, the heteroalkylene is a substituted 18 to 42 membered heteroalkylene. In embodiments, the heteroalkylene is a substituted 24 to 36 membered heteroalkylene. In embodiments, the heteroalkylene comprises an oxygen atom, a phosphorous atom, or a combination thereof. In embodiments, the substituents on the substituted heteroalkylene comprise oxo, —OH, or a combination thereof. In embodiments, the substituted heteroalkylene is:

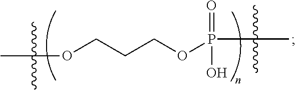

wherein n is an integer from 1 to 10. In embodiments, n is an integer from 4 to 6. In embodiments, n is 5. In embodiments, the heteroalkylene is a substituted 18 to 42 membered heteroalkylene. In embodiments, the heteroalkylene is a substituted 24 to 36 membered heteroalkylene. In embodiments, the heteroalkylene comprises an oxygen atom, a phosphorous atom, or a combination thereof. In embodiments, the substituents on the substituted heteroalkylene comprise oxo, —OH, or a combination thereof. In embodiments, the substituted heteroalkylene is:

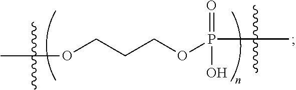

wherein n is an integer from 1 to 10. In embodiments, n is an integer from 4 to 6. In embodiments, n is 5.

Provided herein is a nucleic acid comprising a CpG ODN and SEQ ID NO:11. In embodiments, the CpG ODN is any CpG ODN known in the art. In embodiments, the CpG ODN is Class A CpG ODN (e.g., ODN D19, ODN 1585, ODN 2216, ODN 2336), a Class B CpG ODN (e.g., ODN 1668, ODN 1826, ODN 2006, ODN 2007, ODN BW006, ODN D-SL01), or a Class C CpG ODN (e.g., ODN 2395, ODN M362, ODN D-SL03). In embodiments, the nucleic acid comprises SEQ ID NO:13 and SEQ ID NO:11. In embodiments, SEQ ID NO:13 is bonded to SEQ ID NO: 11. In embodiments, SEQ ID NO:13 is bonded via a linking group to SEQ ID NO:11. In embodiments, the linking group is a bond, a nucleic acid, or a chemical moiety. In embodiments, SEQ ID NO:13 is covalently bonded via a substituted or unsubstituted heteroalkylene to SEQ ID NO:11. In embodiments, SEQ ID NO: 13 is covalently bonded via a substituted or unsubstituted heteroalkylene to SEQ ID NO:11, and SEQ ID NO: 11 is hybridized to SEQ ID NO: 12. In embodiments, SEQ ID NO:11 is a sense strand of STAT3 siRNA and SEQ ID NO: 12 is the complementary antisense strand of STAT3 siRNA. In embodiments, the heteroalkylene is a substituted 18 to 42 membered heteroalkylene. In embodiments, the heteroalkylene is a substituted 24 to 36 membered heteroalkylene. In embodiments, the heteroalkylene comprises an oxygen atom, a phosphorous atom, or a combination thereof. In embodiments, the substituents on the substituted heteroalkylene comprise oxo, —OH, or a combination thereof. In embodiments, the substituted heteroalkylene is:

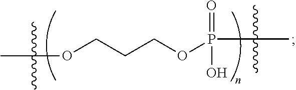

wherein n is an integer from 1 to 10. In embodiments, n is an integer from 4 to 6. In embodiments, n is 5.

In embodiments, the nucleic acid is a CpG ODN-STAT3 siRNA as shown in Table 3. In embodiments, the nucleic acid comprises SEQ ID NO:14. In embodiments, the nucleic acid comprises SEQ ID NO: 15. In embodiments, the nucleic acid comprises SEQ ID NO:6.

TABLE 3

| SEQ | |
|---|---|
| ID NO: | CpG ODN-STAT3 siRNA Sequences (5′→3′) |
| 14 | G*G*T GCA TCG ATG CAG G*G*G*G*G- |
| | xxxxx-UCA GUC GUA UCU UUC UGC AGC |
| | UUC CmGU |
| 15 | G*G*T GCA TCG ATG CAG G*G*G*G*G- |
| | xxxxx-mA*mG*G mAUmG ACU UUmG AmUU |
| | mUCmA ACU |
| 6 | G*G*T GCA TCG ATG CAG G*G*G*G*G- |
| | xxxxx-mA*mG*mG mAmUmG fACfU fUfUmG |
| | AmUmU mUmCmA mAmCmU |

In embodiments, SEQ ID NO:14 is hybridized to SEQ ID NO:8. In particular, the siRNA portion of SEQ ID NO:14 is hybridized to SEQ ID NO:8. In embodiments, SEQ ID NO: 15 is hybridized to SEQ ID NO:10. In particular, the siRNA portion of SEQ ID NO: 15 is hybridized to SEQ ID NO:10. In embodiments, SEQ ID NO:6 is hybridized to SEQ ID NO: 12. In particular, the siRNA portion of SEQ ID NO:6 is hybridized to SEQ ID NO: 12.

For SEQ ID NOS: 14, 15, and 6, each “x” is

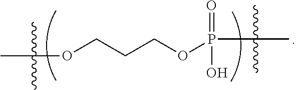

TABLE 4
Legend for Tables 2 and 3

Symbol

| | |
|---|---|
| * | nucleotide having a phosphorothioate modification |
| | * appears after the nucleotide having the modification) |
| dA | adenosine having deoxyribose sugar |
| dG | guanosine having deoxyribose sugar |
| mA | adenosine ribonucleotide with a 2′-O-methyl modification |
| mC | cytidine ribonucleotide with a 2′-O-methyl modification |
| mG | RNA methylated guanosine with a 2′-O-methyl modification |
| mU | RNA methylated uridine with a 2′-O-methyl modification |
| A or rA | unmodified adenosine ribonucleotide |
| C or rC | unmodified cytidine ribonucleotide |
| G or rG | unmodified guanosine ribonucleotide |
| U or rU | unmodified uridine ribonucleotide |
| fA | adenosine ribonucleotide with a 2′-fluoro modification |
| fC | cytidine ribonucleotide with a 2′-fluoro modification |
| fG | guanosine ribonucleotide with a 2′-fluoro modification |
| fU | uridine ribonucleotide with a 2′-fluoro modification |

In embodiments, any of the nucleic acids described herein, including embodiments thereof, can be encapsulated within lipid nanoparticles. In embodiments, any of the nucleic acids described herein, including embodiments thereof, can be encapsulated within the lipid nanoparticles described herein.

### Lipid Nanoparticles

Provided herein are lipid nanoparticles comprising a cationic lipid (e.g., a dilinoleic cationic lipid), a phospholipid, a sterol, and a polyethylene glycol-lipid conjugate (PEG-lipid conjugate). In embodiments, the cationic lipid is DOTAP, DODAC, DODMA, DSDMA, DOTMA, DDAB, DC-Chol, DMRIE, DOSPA, DOGS, CLinDMA, CpLinDMA, DMOBA, DOcarbDAP, DOAP, C12-200, or a mixture of two or more thereof. In embodiments, the cationic lipid is a dilinoleic cationic lipid. In embodiments, the dilinoleic cationic lipid is MC3, an MC3 derivative, DLinDMA, DLin-K-C2-DMA, DLin-K-C3-DMA, DLin-K-C4-DMA, DLin-K6-DMA, DLin-K-MPZ, DLin-K-DMA, DLin-C-DAP, DLin-DAC, DLin-MA, DLinDAP, DLin-S-DMA, DLin-2-DMAP, DLin-TMA, DLin-TAP, DLin-MPZ, DLinAP, DLin-EG-DMA, DLincarbDAP, or a mixture of two or more thereof. In embodiments, the dilinoleic cationic lipid is MC3. In embodiments, the phospholipid is DSPC, DPPC, DOPE, POPC, POPE, POPG, DPPE, DMPE, DSPE, MMPE, DMPE, DEPE, SOPE, EPC, HSPC, or a mixture of two or more thereof. In embodiments, the phospholipid is DSPC, DPPC, DOPE, POPC, POPE, POPG, DPPE, DMPE, DSPE, MMPE, DMPE, DEPE, SOPE, EPC, HSPC, DPPG, or a mixture of two or more thereof. In embodiments, the phospholipid is HSPC. In embodiments, the phospholipid is DPPG. In embodiments, the sterol is cholesterol, cholestanol, cholestanone, cholestenone, coprostanol, cholesteryl-2′-hydroxyethyl ether, cholesteryl-4′-hydroxybutyl ether, or a mixture of two or more thereof. In embodiments, the sterol is cholesterol, cholestanol, cholestanone, cholestenone, coprostanol, cholesteryl-2′-hydroxyethyl ether, cholesteryl-4′-hydroxybutyl ether, cholesteryl hemisuccinate, or a mixture of two or more thereof. In embodiments, the sterol is cholesterol. In embodiments, the sterol is cholesteryl hemisuccinate. In embodiments, the sterol is cholesterol, cholesteryl hemisuccinate, or a mixture thereof. In embodiments, the sterol is cholesterol and cholesteryl hemisuccinate (i.e., a mixture of cholesterol and cholesteryl hemisuccinate). In embodiments, the PEG-lipid conjugate is a polyethylene glycol having a molecular weight from about 1,000 Daltons to about 6,000 Daltons conjugated to a C₁₂-C₂₂ fatty acid lipid. In embodiments, the PEG-lipid conjugate is DMG-PEG, DPPE-PEG, DPG-PEG, DSG-PEG, DSPE-PEG, or a mixture of two or more thereof. In embodiments, the PEG-lipid conjugate is DMG-PEG In embodiments, the PEG-lipid conjugate is DMG-PEG, DPPE-PEG, DPG-PEG, DSG-PEG, DSPE-PEG, or a mixture of two or more thereof, wherein the PEG in each compound has a molecular weight of about 2,000 Daltons. In embodiments, the PEG-lipid conjugate is DMG-PEG2000 (i.e., wherein PEG2000 refers to PEG having a molecular weight of about 2,000 Daltons). In embodiments, the cationic lipid is MC3, the phospholipid is DSPC, the sterol is cholesterol, and the PEG-lipid conjugate is DMG-PEG2000. In embodiments, the cationic lipid is MC3, the phospholipid is DPPG, the sterol is cholesterol, and the PEG-lipid conjugate is DMG-PEG2000. In embodiments, the cationic lipid is MC3, the phospholipid is DSPC, the sterol is cholesterol and cholesteryl hemisuccinate, and the PEG-lipid conjugate is DMG-PEG2000. In embodiments, the lipid nanoparticles are a plurality of lipid nanoparticles.

Provided herein are lipid nanoparticles comprising: (i) about 40 mole % to about 60 mole % of a cationic lipid; (ii) about 5 mole % to about 15 mole % of a phospholipid; (iii) about 25 mole % to about 50 mole % of a sterol; and (iv) about 0.1 mole % to about 4 mole % of a PEG-lipid conjugate. In embodiments, the lipid nanoparticles comprise: (i) about 45 mole % to about 55 mole % of a cationic lipid; (ii) about 5 mole % to about 15 mole % of a phospholipid; (iii) about 33 mole % to about 44 mole % of a sterol; and (iv) about 0.1 mole % to about 3 mole % of a PEG-lipid conjugate. In embodiments, the lipid nanoparticles comprise: (i) about 48 mole % to about 52 mole % of a cationic lipid; (ii) about 8 mole % to about 12 mole % of a phospholipid; (iii) about 36 mole % to about 42 mole % of a sterol; and (iv) about 0.1 mole % to about 2 mole % of a PEG-lipid conjugate. In embodiments, the lipid nanoparticles comprise: (i) about 48 mole % to about 52 mole % of a cationic lipid; (ii) about 8 mole % to about 12 mole % of a phospholipid; (iii) about 36 mole % to about 40 mole % of a sterol; and (iv) about 1 mole % to about 2 mole % of a PEG-lipid conjugate. In embodiments, the lipid nanoparticles comprise: (i) about 50 mole % of a cationic lipid; (ii) about 10 mole % of a phospholipid; (iii) about 38.5 mole % of a sterol; and (iv) about 1.5 mole % of a PEG-lipid conjugate. In embodiments, the lipid nanoparticles comprise: (i) about 48 mole % to about 52 mole % of a cationic lipid; (ii) about 8 mole % to about 12 mole % of a phospholipid; (iii) about 38 mole % to about 42 mole % of a sterol; and (iv) about 0.1 mole % to about 1 mole % of a PEG-lipid conjugate. In embodiments, the lipid nanoparticles comprise: (i) about 50 mole % of a cationic lipid; (ii) about 10 mole % of a phospholipid; (iii) about 39.5 mole % of a sterol; and (iv) about 0.5 mole % of a PEG-lipid conjugate. In embodiments, the cationic lipid is DOTAP, DODAC, DODMA, DSDMA, DOTMA, DDAB, DC-Chol, DMRIE, DOSPA, DOGS, CLinDMA, CpLinDMA, DMOBA, DOcarbDAP, DOAP, C12-200, a dilinoleic cationic lipid, or a mixture of two or more thereof. In embodiments, the phospholipid is DSPC, DPPC, DOPE, POPC, POPE, POPG, DPPE, DMPE, DSPE, MMPE, DMPE, DEPE, SOPE, EPC, HSPC, or a mixture of two or more thereof. In embodiments, the phospholipid is DSPC, DPPC, DOPE, POPC, POPE, POPG, DPPE, DMPE, DSPE, MMPE, DMPE, DEPE, SOPE, EPC, HSPC, DPPG, or a mixture of two or more thereof. In embodiments, the phospholipid is HSPC. In embodiments, the phospholipid is DPPG. In embodiments, the sterol is cholesterol, cholestanol, cholestanone, cholestenone, coprostanol, cholesteryl-2′-hydroxyethyl ether, cholesteryl-4′-hydroxybutyl ether, or a mixture of two or more thereof. In embodiments, the sterol is cholesterol, cholestanol, cholestanone, cholestenone, coprostanol, cholesteryl-2′-hydroxyethyl ether, cholesteryl-4′-hydroxybutyl ether, cholesteryl hemisuccinate, or a mixture of two or more thereof. In embodiments, the sterol is cholesterol. In embodiments, the sterol is cholesteryl hemisuccinate. In embodiments, the sterol is cholesterol, cholesteryl hemisuccinate, or a mixture thereof. In embodiments, the sterol is cholesterol and cholesteryl hemisuccinate (i.e., a mixture of cholesterol and cholesteryl hemisuccinate). In embodiments, the PEG-lipid conjugate is a polyethylene glycol having a molecular weight from about 1,000 Daltons to about 6,000 Daltons conjugated to a C₁₂-C₂₂ fatty acid lipid. In embodiments, the PEG-lipid conjugate is DMG-PEG, DPPE-PEG, DPG-PEG, DSG-PEG, DSPE-PEG, or a mixture of two or more thereof. In embodiments, the PEG-lipid conjugate is DMG-PEG, DPPE-PEG, DPG-PEG, DSG-PEG, DSPE-PEG, or a mixture of two or more thereof, wherein the PEG in each compound has a molecular weight of about 2,000 Daltons. In embodiments, the PEG-lipid conjugate is DMG-PEG. In embodiments, the PEG-lipid conjugate is DMG-PEG2000 ceramide (i.e., wherein PEG2000 refers to PEG having a molecular weight of about 2,000 Daltons). In embodiments, the dilinoleic cationic lipid is MC3, the phospholipid is HSPC, the sterol is cholesterol, and the PEG-lipid conjugate is DMG-PEG2000. In embodiments, the cationic lipid is MC3, the phospholipid is DPPG, the sterol is cholesterol, and the PEG-lipid conjugate is DMG-PEG2000. In embodiments, the cationic lipid is MC3, the phospholipid is DSPC, the sterol is cholesterol and cholesteryl hemisuccinate, and the PEG-lipid conjugate is DMG-PEG2000. In embodiments, the lipid nanoparticles are a plurality of lipid nanoparticles.

Provided herein are lipid nanoparticles comprising: (i) about 40 mole % to about 60 mole % of a dilinoleic cationic lipid; (ii) about 5 mole % to about 15 mole % of a phospholipid; (iii) about 25 mole % to about 50 mole % of a sterol; and (iv) about 0.1 mole % to about 4 mole % of a PEG-lipid conjugate. In embodiments, the lipid nanoparticles comprise: (i) about 45 mole % to about 55 mole % of a dilinoleic cationic lipid; (ii) about 5 mole % to about 15 mole % of a phospholipid; (iii) about 33 mole % to about 44 mole % of a sterol; and (iv) about 0.1 mole % to about 3 mole % of a PEG-lipid conjugate. In embodiments, the lipid nanoparticles comprise: (i) about 48 mole % to about 52 mole % of a dilinoleic cationic lipid; (ii) about 8 mole % to about 12 mole % of a phospholipid; (iii) about 36 mole % to about 42 mole % of a sterol; and (iv) about 0.1 mole % to about 2 mole % of a PEG-lipid conjugate. In embodiments, the lipid nanoparticles comprise: (i) about 48 mole % to about 52 mole % of a dilinoleic cationic lipid; (ii) about 8 mole % to about 12 mole % of a phospholipid; (iii) about 36 mole % to about 40 mole % of a sterol; and (iv) about 1 mole % to about 2 mole % of a PEG-lipid conjugate. In embodiments, the lipid nanoparticles comprise: (i) about 50 mole % of a dilinoleic cationic lipid; (ii) about 10 mole % of a phospholipid; (iii) about 38.5 mole % of a sterol; and (iv) about 1.5 mole % of a PEG-lipid conjugate. In embodiments, the lipid nanoparticles comprise: (i) about 48 mole % to about 52 mole % of a dilinoleic cationic lipid; (ii) about 8 mole % to about 12 mole % of a phospholipid; (iii) about 38 mole % to about 42 mole % of a sterol; and (iv) about 0.1 mole % to about 1 mole % of a PEG-lipid conjugate. In embodiments, the lipid nanoparticles comprise: (i) about 50 mole % of a dilinoleic cationic lipid; (ii) about 10 mole % of a phospholipid; (iii) about 39.5 mole % of a sterol; and (iv) about 0.5 mole % of a PEG-lipid conjugate. In embodiments, the dilinoleic cationic lipid is MC3, an MC3 derivative, DLinDMA, DLin-K-C2-DMA, DLin-K-C3-DMA, DLin-K-C4-DMA, DLin-K6-DMA, DLin-K-MPZ, DLin-K-DMA, DLin-C-DAP, DLin-DAC, DLin-MA, DLinDAP, DLin-S-DMA, DLin-2-DMAP, DLin-TMA, DLin-TAP, DLin-MPZ, DLinAP, DLin-EG-DMA, DLincarbDAP, or a mixture of two or more thereof. In embodiments, the dilinoleic cationic lipid is MC3. In embodiments, the phospholipid is DSPC, DPPC, DOPE, POPC, POPE, POPG, DPPE, DMPE, DSPE, MMPE, DMPE, DEPE, SOPE, EPC, HSPC, or a mixture of two or more thereof. In embodiments, the phospholipid is DSPC, DPPC, DOPE, POPC, POPE, POPG, DPPE, DMPE, DSPE, MMPE, DMPE, DEPE, SOPE, EPC, HSPC, DPPG, or a mixture of two or more thereof. In embodiments, the phospholipid is HSPC. In embodiments, the phospholipid is DPPG. In embodiments, the sterol is cholesterol, cholestanol, cholestanone, cholestenone, coprostanol, cholesteryl-2′-hydroxyethyl ether, cholesteryl-4′-hydroxybutyl ether, or a mixture of two or more thereof. In embodiments, the sterol is cholesterol, cholestanol, cholestanone, cholestenone, coprostanol, cholesteryl-2′-hydroxyethyl ether, cholesteryl-4′-hydroxybutyl ether, cholesteryl hemisuccinate, or a mixture of two or more thereof. In embodiments, the sterol is cholesterol. In embodiments, the sterol is cholesteryl hemisuccinate. In embodiments, the sterol is cholesterol, cholesteryl hemisuccinate, or a mixture thereof. In embodiments, the sterol is cholesterol and cholesteryl hemisuccinate (i.e., a mixture of cholesterol and cholesteryl hemisuccinate). In embodiments, the PEG-lipid conjugate is a polyethylene glycol having a molecular weight from about 1,000 Daltons to about 6,000 Daltons conjugated to a C₁₂-C₂₂ fatty acid lipid. In embodiments, the PEG-lipid conjugate is DMG-PEG, DPPE-PEG, DPG-PEG, DSG-PEG, DSPE-PEG, or a mixture of two or more thereof. In embodiments, the PEG-lipid conjugate is DMG-PEG, DPPE-PEG, DPG-PEG, DSG-PEG, DSPE-PEG, or a mixture of two or more thereof, wherein the PEG in each compound has a molecular weight of about 2,000 Daltons. In embodiments, the PEG-lipid conjugate is DMG-PEG. In embodiments, the PEG-lipid conjugate is DMG-PEG2000 ceramide (i.e., wherein PEG2000 refers to PEG having a molecular weight of about 2,000 Daltons). In embodiments, the dilinoleic cationic lipid is MC3, the phospholipid is HSPC, the sterol is cholesterol, and the PEG-lipid conjugate is DMG-PEG2000. In embodiments, the cationic lipid is MC3, the phospholipid is DPPG, the sterol is cholesterol, and the PEG-lipid conjugate is DMG-PEG2000. In embodiments, the cationic lipid is MC3, the phospholipid is DSPC, the sterol is cholesterol and cholesteryl hemisuccinate, and the PEG-lipid conjugate is DMG-PEG2000. In embodiments, the lipid nanoparticles are a plurality of lipid nanoparticles.

In embodiments of the lipid nanoparticles described herein, the cationic lipid is any cationic lipid known in the art. In embodiments, the cationic lipid is DOTAP, DODAC, DODMA, DSDMA, DOTMA, DDAB, DC-Chol, DMRIE, DOSPA, DOGS, CLinDMA, CpLinDMA, DMOBA, DOcarbDAP, DOAP, C₁₂-200, or a mixture of two or more thereof. In embodiments, the cationic lipid is DOTAP. In embodiments, the cationic lipid is DODAC. In embodiments, the cationic lipid is DODMA. In embodiments, the cationic lipid is DSDMA. In embodiments, the cationic lipid is DOTMA. In embodiments, the cationic lipid is DDAB. In embodiments, the cationic lipid is DC-Chol. In embodiments, the cationic lipid is DMRIE. In embodiments, the cationic lipid is DOSPA. In embodiments, the cationic lipid is DOGS. In embodiments, the cationic lipid is CLinDMA. In embodiments, the cationic lipid is CpLinDMA. In embodiments, the cationic lipid is DMOBA. In embodiments, the cationic lipid is DOcarbDAP. In embodiments, the cationic lipid is DOAP. In embodiments, the cationic lipid is C₁₂-200. In embodiments, the cationic lipid is a dilinoleic cationic lipid. In embodiments, the cationic lipid is not a dilinoleic cationic lipid.

In embodiments of the lipid nanoparticles described herein, the dilinoleic cationic lipid is any known in the art. In embodiments, the dilinoleic cationic lipid is MC3, an MC3 derivative, DLinDMA, DLin-KC2-DMA, DLin-KC3-DMA, DLin-KC4-DMA, DLin-K6-DMA, DLin-K-MPZ, DLin-K-DMA, DLin-C-DAP, DLin-DAC, DLin-MA, DLinDAP, DLin-S-DMA, DLin-2-DMAP, DLin-TMA, DLin-TAP, DLin-MPZ, DLinAP, DLin-EG-DMA, DLincarbDAP, or a mixture of two or more thereof. In embodiments, the dilinoleic cationic lipid is MC3, DLinDMA, DLin-KC2-DMA, DLin-KC3-DMA, DLin-KC4-DMA, DLin-K6-DMA, DLin-K-MPZ, DLin-K-DMA, DLin-C-DAP, DLin-DAC, DLin-MA, DLinDAP, DLin-S-DMA, DLin-2-DMAP, DLin-TMA, DLin-TAP, DLin-MPZ, DLinAP, DLin-EG-DMA, DLincarbDAP, or a mixture of two or more thereof. In embodiments, the dilinoleic cationic lipid is MC3. In embodiments, the dilinoleic cationic lipid is Dlin-KC2-DMA. In embodiments, the dilinoleic cationic lipid is DLinDMA. In embodiments, the dilinoleic cationic lipid is DLin-KC3-DMA. In embodiments, the dilinoleic cationic lipid is DLin-KC4-DMA. In embodiments, the dilinoleic cationic lipid is DLin-K6-DMA. In embodiments, the dilinoleic cationic lipid is DLin-K-MPZ. In embodiments, the dilinoleic cationic lipid is DLin-K-DMA. In embodiments, the dilinoleic cationic lipid is DLin-C-DAP. In embodiments, the dilinoleic cationic lipid is DLin-DAC. In embodiments, the dilinoleic cationic lipid is DLin-MA. In embodiments, the dilinoleic cationic lipid is DLinDAP. In embodiments, the dilinoleic cationic lipid is DLin-S-DMA. In embodiments, the dilinoleic cationic lipid is DLin-2-DMAP. In embodiments, the dilinoleic cationic lipid is Dlin-TMA. In embodiments, the dilinoleic cationic lipid is Dlin-TAP. In embodiments, the dilinoleic cationic lipid is DLin-MPZ. In embodiments, the dilinoleic cationic lipid is DLinAP. In embodiments, the dilinoleic cationic lipid is DLin-EG-DMA. In embodiments, the dilinoleic cationic lipid is DLincarbDAP.

In embodiments of the lipid nanoparticles described herein, the phospholipid is any phospholipid known in the art. In embodiments, the phospholipid is DSPC, DPPC, DOPE, POPC, POPE, POPG, DPPE, DMPE, DSPE, MMPE, DMPE, DEPE, SOPE, EPC, HSPC, or a mixture of two or more thereof. In embodiments, the phospholipid is DSPC, DPPC, DOPE, POPC, POPE, POPG, DPPE, DMPE, DSPE, MMPE, DMPE, DEPE, SOPE, EPC, HSPC, DPPG. or a mixture of two or more thereof. In embodiments, the phospholipid is DSPC. In embodiments, the phospholipid is DPPC. In embodiments, the phospholipid is DOPE. In embodiments, the phospholipid is POPC. In embodiments, the phospholipid is POPE. In embodiments, the phospholipid is POPG. In embodiments, the phospholipid is DPPE. In embodiments, the phospholipid is DMPE. In embodiments, the phospholipid is DSPE. In embodiments, the phospholipid is MMPE. In embodiments, the phospholipid is DMPE. In embodiments, the phospholipid is DEPE. In embodiments, the phospholipid is SOPE. In embodiments, the phospholipid is EPC. In embodiments, the phospholipid is HSPC. In embodiments, the phospholipid is DPPG.

In embodiments of the lipid nanoparticles described herein, the sterol is any sterol known in the art. In embodiments, the sterol is cholesterol, cholestanol, cholestanone, cholestenone, coprostanol, cholesteryl-2′-hydroxyethyl ether, cholesteryl-4′-hydroxybutyl ether, or a mixture of two or more thereof. In embodiments, the sterol is cholesterol, cholestanol, cholestanone, cholestenone, coprostanol, cholesteryl-2′-hydroxyethyl ether, cholesteryl-4′-hydroxybutyl ether, cholesteryl hemisuccinate, or a mixture of two or more thereof. In embodiments, the sterol is a mixture of two compounds selected from the group consisting of cholesterol, cholestanol, cholestanone, cholestenone, coprostanol, cholesteryl-2′-hydroxyethyl ether, cholesteryl-4′-hydroxybutyl ether, and cholesteryl hemisuccinate. In embodiments, the sterol is cholesterol. In embodiments, the sterol is cholestanol. In embodiments, the sterol is cholestanone. In embodiments, the sterol is cholestenone. In embodiments, the sterol is coprostanol. In embodiments, the sterol is cholesteryl-2′-hydroxyethyl ether. In embodiments, the sterol is cholesteryl-4′-hydroxybutyl ether. In embodiments, the sterol is cholesteryl hemisuccinate. In embodiments, the sterol is cholesterol, cholesteryl hemisuccinate, or a mixture thereof. In embodiments, the sterol is cholesterol and cholesteryl hemisuccinate (i.e., a mixture of cholesterol and cholesteryl hemisuccinate).

In embodiments of the lipid nanoparticles described herein, the PEG-lipid conjugate is a polyethylene glycol having a molecular weight from about 1,000 Daltons to about 6,000 Daltons conjugated to a C₁₂-C₂₂ fatty acid lipid. In embodiments, the PEG-lipid conjugate is a polyethylene glycol having a molecular weight from about 1,000 Daltons to about 6,000 Daltons conjugated to a C₁₂-C₂₀ fatty acid lipid. In embodiments, the PEG-lipid conjugate is a polyethylene glycol having a molecular weight from about 1,000 Daltons to about 6,000 Daltons conjugated to a C₁₂-C₁₈ fatty acid lipid. In embodiments, the PEG-lipid conjugate is a polyethylene glycol having a molecular weight from about 1,000 Daltons to about 5,000 Daltons conjugated to a C₁₂-C₂₂ fatty acid lipid. In embodiments, the PEG-lipid conjugate is a polyethylene glycol having a molecular weight from about 1,000 Daltons to about 4,000 Daltons conjugated to a C₁₂-C₂₀ fatty acid lipid. In embodiments, the PEG-lipid conjugate is a polyethylene glycol having a molecular weight from about 1,500 Daltons to about 4,000 Daltons conjugated to a C₁₂-C₁₈ fatty acid lipid. In embodiments, the PEG-lipid conjugate is a polyethylene glycol having a molecular weight from about 1,500 Daltons to about 3,000 Daltons conjugated to a C₁₂-C₁₈ fatty acid lipid. In embodiments, the PEG-lipid conjugate is a polyethylene glycol having a molecular weight from about 1,500 Daltons to about 2,500 Daltons conjugated to a C₁₂ fatty acid lipid. In embodiments, the fatty acid lipid is saturated. In embodiments, the fatty acid lipid is unsaturated. In embodiments, the fatty acid lipid comprises one, two, or three —CH═CH— groups. In embodiments, the fatty acid lipid comprises one —CH═CH— group. In embodiments, the fatty acid lipid comprises two —CH═CH— groups. In embodiments, the PEG-lipid conjugate is DMG-PEG, DPPE-PEG, DPG-PEG, DSG-PEG, DSPE-PEG, or a mixture of two or more thereof. In embodiments, the PEG-lipid conjugate is DMG-PEG. In embodiments, the PEG-lipid conjugate is DPPE-PEG. In embodiments, the PEG-lipid conjugate is DPG-PEG. In embodiments, the PEG-lipid conjugate is DSG-PEG. In embodiments, the PEG-lipid conjugate is DSPE-PEG. In embodiments, the PEG-lipid conjugate is DMG-PEG2000, DPPE-PEG2000, DPG-PEG2000, DSG-PEG2000, DSPE-PEG2000, or a mixture of two or more thereof, wherein PEG2000 refers to PEG having a molecular weight of about 2,000 Daltons. In embodiments, the PEG-lipid conjugate is DMG-PEG2000. In embodiments, the PEG-lipid conjugate is DPPE-PEG2000. In embodiments, the PEG-lipid conjugate is DPG-PEG2000. In embodiments, the PEG-lipid conjugate is DSG-PEG2000. In embodiments, the PEG-lipid conjugate is DSPE-PEG2000.

Provided herein are lipid nanoparticles comprising: (i) about 40 mole % to about 60 mole % of MC3; (ii) about 5 mole % to about 15 mole % of HSPC; (iii) about 25 mole % to about 50 mole % of cholesterol; and (iv) about 0.1 mole % to about 4 mole % of DMG-PEG2000. In embodiments, the lipid nanoparticles comprise: (i) about 45 mole % to about 55 mole % of MC3; (ii) about 5 mole % to about 15 mole % of HSPC; (iii) about 33 mole % to about 44 mole % of cholesterol; and (iv) about 0.1 mole % to about 3 mole % of DMG-PEG2000. In embodiments, the lipid nanoparticles comprise: (i) about 48 mole % to about 52 mole % of MC3; (ii) about 8 mole % to about 12 mole % of HSPC; (iii) about 36 mole % to about 42 mole % of cholesterol; and (iv) about 0.1 mole % to about 2 mole % of DMG-PEG2000.

In embodiments, the lipid nanoparticles comprise: (i) about 48 mole % to about 52 mole % of MC3; (ii) about 8 mole % to about 12 mole % of HSPC; (iii) about 36 mole % to about 40 mole % of cholesterol; and (iv) about 1 mole % to about 2 mole % of DMG-PEG2000. In embodiments, the lipid nanoparticles comprise: (i) about 49 mole % to about 51 mole % of MC3; (ii) about 9 mole % to about 11 mole % of HSPC; (iii) about 37.5 mole % to about 39.5 mole % of cholesterol; and (iv) about 1 mole % to about 2 mole % of DMG-PEG2000. In embodiments, the lipid nanoparticles comprise: (i) about 50 mole % of MC3; (ii) about 10 mole % of HSPC; (iii) about 38.5 mole % of cholesterol; and (iv) about 1.5 mole % of DMG-PEG2000.

In embodiments, the lipid nanoparticles comprise: (i) about 48 mole % to about 52 mole % of MC3; (ii) about 8 mole % to about 12 mole % of HSPC; (iii) about 38 mole % to about 42 mole % of cholesterol; and (iv) about 0.1 mole % to about 1 mole % of DMG-PEG2000. In embodiments, the lipid nanoparticles comprise: (i) about 49 mole % to about 51 mole % of MC3; (ii) about 9 mole % to about 11 mole % of HSPC; (iii) about 38.5 mole % to about 40.5 mole % of cholesterol; and (iv) about 0.2 mole % to about 1 mole % of DMG-PEG2000. In embodiments, the lipid nanoparticles comprise: (i) about 50 mole % of MC3; (ii) about 10 mole % of HSPC; (iii) about 39.5 mole % of cholesterol; and (iv) about 0.5 mole % of DMG-PEG2000. In embodiments, the lipid nanoparticles are a plurality of lipid nanoparticles.

Provided herein are lipid nanoparticles comprising: (i) about 40 mole % to about 60 mole % of MC3; (ii) about 5 mole % to about 15 mole % of DPPG; (iii) about 25 mole % to about 50 mole % of cholesterol; and (iv) about 0.1 mole % to about 4 mole % of DMG-PEG2000. In embodiments, the lipid nanoparticles comprise: (i) about 45 mole % to about 55 mole % of MC3; (ii) about 5 mole % to about 15 mole % of DPPG; (iii) about 33 mole % to about 44 mole % of cholesterol; and (iv) about 0.1 mole % to about 3 mole % of DMG-PEG2000. In embodiments, the lipid nanoparticles comprise: (i) about 48 mole % to about 52 mole % of MC3; (ii) about 8 mole % to about 12 mole % of DPPG; (iii) about 36 mole % to about 42 mole % of cholesterol; and (iv) about 0.1 mole % to about 2 mole % of DMG-PEG2000.

In embodiments, the lipid nanoparticles comprise: (i) about 48 mole % to about 52 mole % of MC3; (ii) about 8 mole % to about 12 mole % of DPPG; (iii) about 36 mole % to about 40 mole % of cholesterol; and (iv) about 1 mole % to about 2 mole % of DMG-PEG2000. In embodiments, the lipid nanoparticles comprise: (i) about 49 mole % to about 51 mole % of MC3; (ii) about 9 mole % to about 11 mole % of DPPG; (iii) about 37.5 mole % to about 39.5 mole % of cholesterol; and (iv) about 1 mole % to about 2 mole % of DMG-PEG2000. In embodiments, the lipid nanoparticles comprise: (i) about 50 mole % of MC3; (ii) about 10 mole % of DPPG; (iii) about 38.5 mole % of cholesterol; and (iv) about 1.5 mole % of DMG-PEG2000.

In embodiments, the lipid nanoparticles comprise: (i) about 48 mole % to about 52 mole % of MC3; (ii) about 8 mole % to about 12 mole % of DPPG; (iii) about 38 mole % to about 42 mole % of cholesterol; and (iv) about 0.1 mole % to about 1 mole % of DMG-PEG2000. In embodiments, the lipid nanoparticles comprise: (i) about 49 mole % to about 51 mole % of MC3; (ii) about 9 mole % to about 11 mole % of DPPG; (iii) about 38.5 mole % to about 40.5 mole % of cholesterol; and (iv) about 0.2 mole % to about 1 mole % of DMG-PEG2000. In embodiments, the lipid nanoparticles comprise: (i) about 50 mole % of MC3; (ii) about 10 mole % of DPPG; (iii) about 39.5 mole % of cholesterol; and (iv) about 0.5 mole % of DMG-PEG2000. In embodiments, the lipid nanoparticles are a plurality of lipid nanoparticles.

Provided herein are lipid nanoparticles comprising: (i) about 40 mole % to about 60 mole % of MC3; (ii) about 5 mole % to about 15 mole % of HSPC; (iii) about 25 mole % to about 50 mole % of a mixture of cholesterol and cholesteryl hemisuccinate; and (iv) about 0.1 mole % to about 4 mole % of DMG-PEG2000. In embodiments, the lipid nanoparticles comprise: (i) about 45 mole % to about 55 mole % of MC3; (ii) about 5 mole % to about 15 mole % of HSPC; (iii) about 33 mole % to about 44 mole % of a mixture of cholesterol and cholesteryl hemisuccinate; and (iv) about 0.1 mole % to about 3 mole % of DMG-PEG2000. In embodiments, the lipid nanoparticles comprise: (i) about 48 mole % to about 52 mole % of MC3; (ii) about 8 mole % to about 12 mole % of HSPC; (iii) about 36 mole % to about 42 mole % of a mixture of cholesterol and cholesteryl hemisuccinate; and (iv) about 0.1 mole % to about 2 mole % of DMG-PEG2000. In embodiments, the molar ratio of cholesterol to cholesteryl hemisuccinate is from about 3:1 to about 1:3. In embodiments, the molar ratio of cholesterol to cholesteryl hemisuccinate is from about 2:1 to about 1:2. In embodiments, the molar ratio of cholesterol to cholesteryl hemisuccinate is from about 1.8:1 to about 1:1.8. In embodiments, the molar ratio of cholesterol to cholesteryl hemisuccinate is from about 1.5:1 to about 1:1.5. In embodiments, the molar ratio of cholesterol to cholesteryl hemisuccinate is from about 1.2:1 to about 1:1.2. In embodiments, the molar ratio of cholesterol to cholesteryl hemisuccinate is from about 1.1:1 to about 1:1.1. In embodiments, the molar ratio of cholesterol to cholesteryl hemisuccinate is from about 1:1 to about 1:2. In embodiments, the molar ratio of cholesterol to cholesteryl hemisuccinate is from about 1:1 to about 1:1.5. In embodiments, the molar ratio of cholesterol to cholesteryl hemisuccinate is from about 1:1 to about 1:1.4. In embodiments, the molar ratio of cholesterol to cholesteryl hemisuccinate is from about 1:1 to about 1:1.3. In embodiments, the molar ratio of cholesterol to cholesteryl hemisuccinate is from about 1:1 to about 1:1.2. In embodiments, the molar ratio of cholesterol to cholesteryl hemisuccinate is from about 1:1 to about 1:1.1. In embodiments, the molar ratio of cholesterol to cholesteryl hemisuccinate is about 1:1. In embodiments, the molar ratio of cholesterol to cholesteryl hemisuccinate is about 1:1.1. In embodiments, the molar ratio of cholesterol to cholesteryl hemisuccinate is about 1:1.08. In embodiments, the molar ratio of cholesterol to cholesteryl hemisuccinate is about 1:2.

In embodiments, the lipid nanoparticles comprise: (i) about 48 mole % to about 52 mole % of MC3; (ii) about 8 mole % to about 12 mole % of HSPC; (iii) about 36 mole % to about 40 mole % of a mixture of cholesterol and cholesteryl hemisuccinate; and (iv) about 1 mole % to about 2 mole % of DMG-PEG2000. In embodiments, the lipid nanoparticles comprise: (i) about 49 mole % to about 51 mole % of MC3; (ii) about 9 mole % to about 11 mole % of HSPC; (iii) about 37.5 mole % to about 39.5 mole % of a mixture of cholesterol and cholesteryl hemisuccinate; and (iv) about 1 mole % to about 2 mole % of DMG-PEG2000. In embodiments, the lipid nanoparticles comprise: (i) about 50 mole % of MC3; (ii) about 10 mole % of HSPC; (iii) about 38.5 mole % of a mixture of cholesterol and cholesteryl hemisuccinate; and (iv) about 1.5 mole % of DMG-PEG2000. In embodiments, the molar ratio of cholesterol to cholesteryl hemisuccinate is from about 3:1 to about 1:3. In embodiments, the molar ratio of cholesterol to cholesteryl hemisuccinate is from about 2:1 to about 1:2. In embodiments, the molar ratio of cholesterol to cholesteryl hemisuccinate is from about 1.8:1 to about 1:1.8. In embodiments, the molar ratio of cholesterol to cholesteryl hemisuccinate is from about 1.5:1 to about 1:1.5. In embodiments, the molar ratio of cholesterol to cholesteryl hemisuccinate is from about 1.2:1 to about 1:1.2. In embodiments, the molar ratio of cholesterol to cholesteryl hemisuccinate is from about 1.1:1 to about 1:1.1. In embodiments, the molar ratio of cholesterol to cholesteryl hemisuccinate is from about 1:1 to about 1:2. In embodiments, the molar ratio of cholesterol to cholesteryl hemisuccinate is from about 1:1 to about 1:1.5. In embodiments, the molar ratio of cholesterol to cholesteryl hemisuccinate is from about 1:1 to about 1:1.4. In embodiments, the molar ratio of cholesterol to cholesteryl hemisuccinate is from about 1:1 to about 1:1.3. In embodiments, the molar ratio of cholesterol to cholesteryl hemisuccinate is from about 1:1 to about 1:1.2. In embodiments, the molar ratio of cholesterol to cholesteryl hemisuccinate is from about 1:1 to about 1:1.1. In embodiments, the molar ratio of cholesterol to cholesteryl hemisuccinate is about 1:1. In embodiments, the molar ratio of cholesterol to cholesteryl hemisuccinate is about 1:1.1. In embodiments, the molar ratio of cholesterol to cholesteryl hemisuccinate is about 1:1.08. In embodiments, the molar ratio of cholesterol to cholesteryl hemisuccinate is about 1:2. In embodiments, the lipid nanoparticles comprise: (i) about 50 mole % of MC3; (ii) about 10 mole % of HSPC; (iiia) about 18.5 mole % of cholesterol; (iiib) about 20 mole % of cholesteryl hemisuccinate; and (iv) about 1.5 mole % of DMG-PEG2000.

In embodiments, the lipid nanoparticles comprise: (i) about 48 mole % to about 52 mole % of MC3; (ii) about 8 mole % to about 12 mole % of HSPC; (iii) about 38 mole % to about 42 mole % of a mixture of cholesterol and cholesteryl hemisuccinate; and (iv) about 0.1 mole % to about 1 mole % of DMG-PEG2000. In embodiments, the lipid nanoparticles comprise: (i) about 49 mole % to about 51 mole % of MC3; (ii) about 9 mole % to about 11 mole % of HSPC; (iii) about 38.5 mole % to about 40.5 mole % of a mixture of cholesterol and cholesteryl hemisuccinate; and (iv) about 0.2 mole % to about 1 mole % of DMG-PEG2000. In embodiments, the lipid nanoparticles comprise: (i) about 50 mole % of MC3; (ii) about 10 mole % of HSPC; (iii) about 39.5 mole % of a mixture of cholesterol and cholesteryl hemisuccinate; and (iv) about 0.5 mole % of DMG-PEG2000. In embodiments, the lipid nanoparticles are a plurality of lipid nanoparticles. In embodiments, the molar ratio of cholesterol to cholesteryl hemisuccinate is from about 3:1 to about 1:3. In embodiments, the molar ratio of cholesterol to cholesteryl hemisuccinate is from about 2:1 to about 1:2. In embodiments, the molar ratio of cholesterol to cholesteryl hemisuccinate is from about 1.8:1 to about 1:1.8. In embodiments, the molar ratio of cholesterol to cholesteryl hemisuccinate is from about 1.5:1 to about 1:1.5. In embodiments, the molar ratio of cholesterol to cholesteryl hemisuccinate is from about 1.2:1 to about 1:1.2. In embodiments, the molar ratio of cholesterol to cholesteryl hemisuccinate is from about 1.1:1 to about 1:1.1. In embodiments, the molar ratio of cholesterol to cholesteryl hemisuccinate is from about 1:1 to about 1:2. In embodiments, the molar ratio of cholesterol to cholesteryl hemisuccinate is from about 1:1 to about 1:1.5. In embodiments, the molar ratio of cholesterol to cholesteryl hemisuccinate is from about 1:1 to about 1:1.4. In embodiments, the molar ratio of cholesterol to cholesteryl hemisuccinate is from about 1:1 to about 1:1.3. In embodiments, the molar ratio of cholesterol to cholesteryl hemisuccinate is from about 1:1 to about 1:1.2. In embodiments, the molar ratio of cholesterol to cholesteryl hemisuccinate is from about 1:1 to about 1:1.1. In embodiments, the molar ratio of cholesterol to cholesteryl hemisuccinate is about 1:1. In embodiments, the molar ratio of cholesterol to cholesteryl hemisuccinate is about 1:1.1. In embodiments, the molar ratio of cholesterol to cholesteryl hemisuccinate is about 1:1.08. In embodiments, the molar ratio of cholesterol to cholesteryl hemisuccinate is about 1:2.

Provided herein are lipid nanoparticles comprising: (i) about 40 mole % to about 60 mole % of MC3; (ii) about 5 mole % to about 15 mole % of HSPC; (iii) about 12 mole % to about 25 mole % of cholesterol; (iv) about 12 mole % to about 25 mole % of cholesteryl hemisuccinate; and (v) about 0.1 mole % to about 4 mole % of DMG-PEG2000. In embodiments, the lipid nanoparticles comprise: (i) about 48 mole % to about 52 mole % of MC3; (ii) about 8 mole % to about 12 mole % of HSPC; (iii) about 16.5 mole % to about 20.5 mole % of cholesterol, (iv) about 18 mol % to about 22 mol % of cholesteryl hemisuccinate; and (v) about 0.1 mole % to about 1 mole % of DMG-PEG2000. In embodiments, the lipid nanoparticles comprise: (i) about 49 mole % to about 51 mole % of MC3; (ii) about 9 mole % to about 11 mole % of HSPC; (iii) about 17.5 mole % to about 19.5 mole % of cholesterol, (iv) about 19 mol % to about 21 mol % of cholesteryl hemisuccinate; and (v) about 0.2 mole % to about 1 mole % of DMG-PEG2000. In embodiments, the lipid nanoparticles comprise: (i) about 50 mole % of MC3; (ii) about 10 mole % of HSPC; (iiia) about 18.5 mole % of cholesterol; (iiib) about 20 mole % of cholesteryl hemisuccinate; and (iv) about 1.5 mole % of DMG-PEG2000.

The lipid nanoparticles (or plurality of lipid nanoparticles) described herein typically have an average size (e.g., mean diameter) from about 10 nm to about 200 nm, from about 20 nm to about 190 nm, from about 30 nm to about 175 nm, from about 40 nm to about 160 nm, from about 50 nm to about 150 nm, or from about 60 nm to about 140 nm. In embodiments, the lipid nanoparticles described herein have an average size from about 30 nm to about 130 nm. In embodiments, the lipid nanoparticles have an average size from about 35 nm to about 125 nm. In embodiments, the lipid nanoparticles have an average size from about 40 nm to about 120 nm. In embodiments, the lipid nanoparticles have an average size from about 45 nm to about 115 nm. In embodiments, the lipid nanoparticles have an average size from about 50 nm to about 110 nm. In embodiments, the lipid nanoparticles have an average size from about 55 nm to about 105 nm. In embodiments, the lipid nanoparticles have an average size from about 60 nm to about 105 nm. In embodiments, the lipid nanoparticles have an average size from about 60 nm to about 100 nm. In embodiments, the lipid nanoparticles have an average size from about 65 nm to about 95 nm. In embodiments, the lipid nanoparticles have an average size from about 70 nm to about 90 nm. In embodiments, the lipid nanoparticles have an average size from about 75 nm to about 85 nm. In embodiments, the lipid nanoparticles have an average size from about 75 nm to about 80 nm. In embodiments, the lipid nanoparticles have an average size from about 70 nm to about 110 nm. In embodiments, the lipid nanoparticles have an average size from about 70 nm to about 105 nm. In embodiments, the lipid nanoparticles have an average size from about 70 nm to about 100 nm. In embodiments, the lipid nanoparticles have an average size from about 90 nm to about 110 nm. In embodiments, the lipid nanoparticles have an average size from about 95 nm to about 105 nm. In embodiments, the lipid nanoparticles have an average size from about 95 nm to about 100 nm. In embodiments, the lipid nanoparticles have an average size of about 70 nm. In embodiments, the lipid nanoparticles have an average size of about 75 nm. In embodiments, the lipid nanoparticles have an average size of about 80 nm. In embodiments, the lipid nanoparticles have an average size of about 85 nm. In embodiments, the lipid nanoparticles have an average size of about 90 nm. In embodiments, the lipid nanoparticles have an average size of about 95 nm. In embodiments, the lipid nanoparticles have an average size of about 100 nm. In embodiments, lipid nanoparticles refers to a plurality of lipid nanoparticles.

“Zeta potential” is a measure of the effective electric charge on the nanoparticle surface. The magnitude of the zeta potential provides information about particle stability, with particles with higher magnitude zeta potentials exhibiting increased stability due to a larger electrostatic repulsion between particles. In embodiments, the lipid nanoparticles described herein have a zeta potential from about −1 mV to about −50 mV. In embodiments, the lipid nanoparticles described herein have a zeta potential from about −1 mV to about −45 mV. In embodiments, the lipid nanoparticles described herein have a zeta potential from about −5 mV to about −40 mV. In embodiments, the lipid nanoparticles described herein have a zeta potential from about −5 mV to about −35 mV. In embodiments, the lipid nanoparticles described herein have a zeta potential from about −8 mV to about −26 mV. In embodiments, the lipid nanoparticles described herein have a zeta potential from about −10 mV to about −26 mV. In embodiments, the lipid nanoparticles have a zeta potential from about −11 mV to about −25 mV. In embodiments, the lipid nanoparticles have a zeta potential from about −12 mV to about −24 mV. In embodiments, the lipid nanoparticles have a zeta potential from about −13 mV to about −23 mV. In embodiments, the lipid nanoparticles have a zeta potential from about −14 mV to about −22 mV. In embodiments, the lipid nanoparticles have a zeta potential from about −15 mV to about −21 mV. In embodiments, the lipid nanoparticles have a zeta potential from about −16 mV to about −20 mV. In embodiments, the lipid nanoparticles have a zeta potential from about −17 mV to about −20 mV. In embodiments, the lipid nanoparticles have a zeta potential from about −17 mV to about −19 mV. In embodiments, the lipid nanoparticles have a zeta potential from about −18 mV to about −19 mV. In embodiments, the lipid nanoparticles have a zeta potential from about −19 mV to about −20 mV. In embodiments, the lipid nanoparticles have a zeta potential of about −12 mV. In embodiments, the lipid nanoparticles have a zeta potential of about −13 mV. In embodiments, the lipid nanoparticles have a zeta potential of about −14 mV. In embodiments, the lipid nanoparticles have a zeta potential of about −15 mV. In embodiments, the lipid nanoparticles have a zeta potential of about −16 mV. In embodiments, the lipid nanoparticles have a zeta potential of about −17 mV. In embodiments, the lipid nanoparticles have a zeta potential of about −18 mV. In embodiments, the lipid nanoparticles have a zeta potential of about −19 mV. In embodiments, the lipid nanoparticles have a zeta potential of about −20 mV. In embodiments, the lipid nanoparticles have a zeta potential of about −21 mV. In embodiments, the lipid nanoparticles have a zeta potential of about −22 mV. In embodiments, the lipid nanoparticles have a zeta potential of about −23 mV. In embodiments, the lipid nanoparticles have a zeta potential of about −24 mV.

### Lipid Nanoparticles Encapsulating Nucleic Acids

In embodiments, the disclosure provides a lipid nanoparticle comprising a nucleic acid encapsulated within the lipid nanoparticle. For purposes of the disclosure, the phrase “lipid nanoparticles comprises a nucleic acid” is equivalent to the phrase “lipid nanoparticle comprises a nucleic acid encapsulated within the lipid nanoparticle.” In embodiments, the nucleic acid is any nucleic acid described herein, including embodiments thereof. In embodiments, the lipid nanoparticles comprise DNA. In embodiments, the lipid nanoparticles comprise RNA. In embodiments, the lipid nanoparticles comprise siRNA. In embodiments, the lipid nanoparticles comprise STAT3 siRNA. In embodiments, the lipid nanoparticles comprise a CpG ODN attached to an antisense strand of STAT3 siRNA. In embodiments, the lipid nanoparticles comprise a CpG ODN attached to an antisense strand of STAT3 siRNA, wherein the antisense strand of STAT3 siRNA is hybridized to a sense strand of siRNA. In embodiments, the lipid nanoparticles comprise a CpG ODN attached to a sense strand of STAT3 siRNA. In embodiments, the lipid nanoparticles comprise a CpG ODN attached to a sense strand of STAT3 siRNA, wherein the sense strand of STAT3 siRNA is hybridized to an antisense strand of siRNA. In embodiments, the lipid nanoparticles comprise miRNA. In embodiments, the lipid nanoparticles comprise mRNA.

In embodiments, the lipid nanoparticles comprise the nucleic acid having any one of SEQ ID NOS: 7-12 and 14-16. In embodiments, the lipid nanoparticles comprise the nucleic acid having any one of SEQ ID NOS: 7-12. In embodiments, the lipid nanoparticles comprise the nucleic acid having SEQ ID NO:7. In embodiments, the lipid nanoparticles comprise the nucleic acid having SEQ ID NO:8. In embodiments, the lipid nanoparticles comprise the nucleic acid having SEQ ID NO:9. In embodiments, the lipid nanoparticles comprise the nucleic acid having SEQ ID NO:10. In embodiments, the lipid nanoparticles comprise the nucleic acid having SEQ ID NO: 11. In embodiments, the lipid nanoparticles comprise the nucleic acid having SEQ ID NO: 12. In embodiments, the lipid nanoparticles comprise the nucleic acid having SEQ ID NO:7, wherein SEQ ID NO:7 is hybridized to SEQ ID NO:8. In embodiments, the lipid nanoparticles comprise the nucleic acid having SEQ ID NO:9, wherein SEQ ID NO:9 is hybridized to SEQ ID NO: 10. In embodiments, the lipid nanoparticles comprise the nucleic acid having SEQ ID NO: 11, wherein SEQ ID NO: 11 is hybridized to SEQ ID NO: 12.

In embodiments, the lipid nanoparticles comprise the nucleic acid having any one of SEQ ID NOS: 14-16. In embodiments, the lipid nanoparticles comprise the nucleic acid having SEQ ID NO:14. In embodiments, the lipid nanoparticles comprise the nucleic acid having SEQ ID NO: 14, wherein SEQ ID NO: 14 is hybridized to SEQ ID NO:8. In embodiments, the lipid nanoparticles comprise the nucleic acid having SEQ ID NO: 15. In embodiments, the lipid nanoparticles comprise the nucleic acid having SEQ ID NO: 15, wherein SEQ ID NO:15 is hybridized to SEQ ID NO:10. In embodiments, the lipid nanoparticles comprise the nucleic acid having SEQ ID NO:6. In embodiments, the lipid nanoparticles comprise the nucleic acid having SEQ ID NO:6, wherein SEQ ID NO:6 is hybridized to SEQ ID NO: 12.

In embodiments, the lipid nanoparticles comprise the nucleic acid having SEQ ID NO: 16 or SEQ ID NO:17. In embodiments, the lipid nanoparticles comprise the nucleic acid having SEQ ID NO: 16. In embodiments, the lipid nanoparticles comprise the nucleic acid having SEQ ID NO: 17. In embodiments, the lipid nanoparticles comprise the nucleic acid having SEQ ID NO: 16 hybridized to SEQ ID NO:17.

The term “N/P” or “N/P ratio” refers to the ratio of positively-chargeable polymer amine (N=nitrogen) groups to negatively-charged nucleic acid phosphate (P) groups in a lipid encapsulated nanoparticle.

In embodiments of the lipid nanoparticles having a nucleic acid encapsulated therein, the N/P ratio is from about 1:1 to about 15:1. In embodiments, the N/P ratio is from about 1:1 to about 10:1. In embodiments, the N/P ratio is from about 2:1 to about 8:1. In embodiments, the N/P ratio is from about 2:1 to about 7:1. In embodiments, the N/P ratio is from about 3:1 to about 6:1. In embodiments, the N/P ratio is from about 2:1 to about 4:1. In embodiments, the N/P ratio is from about 5:1 to about 7:1. In embodiments, the N/P ratio is about 1:1. In embodiments, the N/P ratio is about 2:1. In embodiments, the N/P ratio is about 3:1. In embodiments, the N/P ratio is about 4:1. In embodiments, the N/P ratio is about 5:1. In embodiments, the N/P ratio is about 6:1. In embodiments, the N/P ratio is about 7:1. In embodiments, the N/P ratio is about 8:1. In embodiments, the N/P ratio is about 9:1. In embodiments, the N/P ratio is about 10:1.

### Pharmaceutical Compositions

Provided herein are pharmaceutical compositions comprising a plurality of lipid nanoparticles which comprise nucleic acids encapsulated therein (e.g., RNA, mRNA, miRNA, siRNA, DNA, CpG ODN, CpG-ODN linked to siRNA) and a pharmaceutically acceptable excipient.

“Pharmaceutically acceptable excipient” and “pharmaceutically acceptable carrier” refer to a substance that aids the administration of an active agent to and absorption by a subject and can be included in the compositions of the disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions, alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful.

Solutions of the nucleic acids or lipid nanoparticles containing nucleic acids can be prepared in water suitably mixed with a lipid or surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions. The intranasal composition can be a spray, aerosol, or inhalant. The inhalable composition can be a spray, aerosol, or inhalant. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known in the art.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In embodiments, oral pharmaceutical compositions will comprise an inert diluent or edible carrier, or they may be enclosed in hard or soft shell gelatin capsule, or they may be compressed into tablets, or they may be incorporated directly with the food. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may be between about 1 to about 75% of the weight of the unit. The amount of nucleic acids in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the nucleic acids in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. Dimethyl sulfoxide can be used as solvent for extremely rapid penetration, delivering high concentrations of the active agents to a small area.

The formulations of nucleic acids (e.g., RNA, mRNA, miRNA, siRNA, DNA, CpG ODN, CpG-ODN linked to siRNA), lipid nanoparticles containing nucleic acids (e.g., RNA, mRNA, miRNA, siRNA, DNA, CpG ODN, CpG-ODN linked to siRNA) can be presented in unit-dose or multi-dose sealed containers, such as nebulizers, ventilators, ampules, and vials. Thus, the composition can be in unit dosage form. In such form the preparation is subdivided into unit doses containing appropriate quantities of nucleic acids or lipid nanoparticles containing nucleic acids. Thus, the compositions can be administered in a variety of unit dosage forms depending upon the method of administration. For example, unit dosage forms suitable for oral administration include, but are not limited to, powder, tablets, pills, capsules and lozenges.

The nucleic acids (e.g., RNA, mRNA, miRNA, siRNA, DNA, CpG ODN, CpG-ODN linked to siRNA), lipid nanoparticles containing nucleic acids (e.g., RNA, mRNA, miRNA, siRNA, DNA, CpG ODN, CpG-ODN linked to siRNA), and pharmaceutical compositions can be administered to the patient in any manner as described herein. In embodiments, the nucleic acids, lipid nanoparticles, and pharmaceutical compositions are administered parenterally to a patient. In embodiments, the nucleic acids, lipid nanoparticles, and pharmaceutical compositions are administered intravenously to a patient. In embodiments, the nucleic acids, lipid nanoparticles, and pharmaceutical compositions are administered subcutaneously to a patient. In embodiments, the nucleic acids, lipid nanoparticles, and pharmaceutical compositions are administered intranodally to a patient. In embodiments, the nucleic acids, lipid nanoparticles, and pharmaceutical compositions are administered intratumorally to a patient.

### Methods

In embodiments, the disclosure provides methods of treating cancer in a subject in need thereof by administering to the subject an effective amount of a lipid nanoparticle comprising a nucleic acid (e.g., RNA, mRNA, miRNA, siRNA, DNA, CpG ODN, CpG-ODN linked to siRNA) encapsulated therein. In embodiments, the disclosure provides methods of treating cancer in a subject in need thereof by administering to the subject an effective amount of a plurality of lipid nanoparticles comprising a nucleic acid (e.g., RNA, mRNA, miRNA, siRNA, DNA, CpG ODN, CpG-ODN linked to siRNA) encapsulated therein. In embodiments, the disclosure provides methods of treating cancer in a subject in need thereof by administering to the subject an effective amount of a pharmaceutical compositions which comprise the lipid nanoparticles comprising a nucleic acid (e.g., RNA, mRNA, miRNA, siRNA, DNA, CpG ODN, CpG-ODN linked to siRNA) encapsulated therein, and a pharmaceutically acceptable excipient.

In embodiments, the disclosure provides methods of treating cancer in a subject in need thereof by administering to the subject an effective amount of a nucleic acid or a pharmaceutical composition comprising the nucleic acid. In embodiments, the disclosure provides methods of treating cancer in a subject in need thereof by administering to the subject an effective amount of a nucleic acid or a pharmaceutical composition comprising the nucleic acid; wherein the nucleic acid comprises any one of SEQ ID NOS: 9-12. In embodiments, the disclosure provides methods of treating cancer in a subject in need thereof by administering to the subject an effective amount of a nucleic acid or a pharmaceutical composition comprising the nucleic acid; wherein the nucleic acid comprises SEQ ID NO:13 bonded to SEQ ID NO:9. In embodiments, the disclosure provides methods of treating cancer in a subject in need thereof by administering to the subject an effective amount of a nucleic acid or a pharmaceutical composition comprising the nucleic acid; wherein the nucleic acid comprises SEQ ID NO: 13 bonded to SEQ ID NO:9, and wherein SEQ ID NO:9 is hybridized to SEQ ID NO:10. In embodiments, the disclosure provides methods of treating cancer in a subject in need thereof by administering to the subject an effective amount of a nucleic acid or a pharmaceutical composition comprising the nucleic acid; wherein the nucleic acid comprises SEQ ID NO:13 bonded to SEQ ID NO:11. In embodiments, the disclosure provides methods of treating cancer in a subject in need thereof by administering to the subject an effective amount of a nucleic acid or a pharmaceutical composition comprising the nucleic acid; wherein the nucleic acid comprises SEQ ID NO:13 bonded to SEQ ID NO:11 is hybridized to SEQ ID NO:12. In embodiments, the disclosure provides methods of treating cancer in a subject in need thereof by administering to the subject an effective amount of a nucleic acid or a pharmaceutical composition comprising the nucleic acid; wherein the nucleic acid comprises SEQ ID NO:15 or 16. In embodiments, the disclosure provides methods of treating cancer in a subject in need thereof by administering to the subject an effective amount of a nucleic acid or a pharmaceutical composition comprising the nucleic acid; wherein the nucleic acid comprises SEQ ID NO:15. In embodiments, the disclosure provides methods of treating cancer in a subject in need thereof by administering to the subject an effective amount of a nucleic acid or a pharmaceutical composition comprising the nucleic acid; wherein the nucleic acid comprises SEQ ID NO:15, wherein SEQ ID NO:15 is hybridized to SEQ ID NO: 10. In embodiments, the disclosure provides methods of treating cancer in a subject in need thereof by administering to the subject an effective amount of a nucleic acid or a pharmaceutical composition comprising the nucleic acid; wherein the nucleic acid comprises SEQ ID NO:6. In embodiments, the disclosure provides methods of treating cancer in a subject in need thereof by administering to the subject an effective amount of a nucleic acid or a pharmaceutical composition comprising the nucleic acid; wherein the nucleic acid comprises SEQ ID NO:6, wherein SEQ ID NO:6 is hybridized to SEQ ID NO:12. In embodiments, the disclosure provides methods of treating cancer in a subject in need thereof by administering to the subject an effective amount of a nucleic acid or a pharmaceutical composition comprising the nucleic acid; wherein the nucleic acid comprises SEQ ID NO: 16, SEQ ID NO:17, or SEQ ID NO:16 hybridized to SEQ ID NO:17.

In embodiments, the cancer is lymphoma. As used herein, “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

In embodiments, the cancer is B cell lymphoma. In embodiments, the lymphoma is diffuse large B cell lymphoma. In embodiments, the cancer is follicular lymphoma, chronic lymphocytic lymphoma (CLL), small lymphocytic lymphoma (SLL), mantle cell lymphoma, marginal zone lymphoma, Burkitt lymphoma, lymphoplasmacytic lymphoma, B cell non-Hodgkin lymphoma, or primary mediastinal large B-cell lymphoma. In embodiments, the cancer is follicular lymphoma. In embodiments, the cancer is mantle cell lymphoma. In embodiments, the cancer is marginal zone lymphoma. In embodiments, the cancer is small lymphocytic lymphoma. In embodiments, the cancer is B cell non-Hodgkin lymphoma. In embodiments, the cancer is recurrent B cell non-Hodgkin lymphoma, recurrent diffuse large B cell lymphoma, recurrent Grade 1 follicular lymphoma, recurrent Grade 2 follicular lymphoma, recurrent Grade 3 follicular lymphoma, recurrent Grade 3a follicular lymphoma, recurrent Grade 3b follicular lymphoma, recurrent mantle cell lymphoma, recurrent marginal zone lymphoma, recurrent small lymphocytic lymphoma, refractory B cell non-Hodgkin lymphoma, refractory diffuse large B cell lymphoma, refractory Grade 1 follicular lymphoma, refractory Grade 2 follicular lymphoma, refractory Grade 3 follicular lymphoma, refractory Grade 3a follicular lymphoma, refractory mantle cell lymphoma, refractory marginal zone lymphoma, refractory small lymphocytic lymphoma, or refractory Grade 3b follicular lymphoma.

In embodiments, the cancer is leukemia. The term “leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia. In embodiments, the cancer is myeloid leukemia. In embodiments, the cancer is chronic myeloid leukemia. In embodiments, the cancer is acute myeloid leukemia.

In embodiments, the cancer is lymphoma or leukemia. In embodiments, the cancer is B cell lymphoma or myeloid leukemia. In embodiments, the cancer is lymphoma, leukemia, glioma, a head and neck cancer, or prostate cancer. In embodiments, the cancer is B cell lymphoma, myeloid leukemia, glioma, a head and neck cancer, or prostate cancer. In embodiments, the cancer is B cell lymphoma. In embodiments, the cancer is myeloid leukemia. In embodiments, the cancer is acute myeloid leukemia.

In embodiments, the cancer is a solid cancer. In embodiments, the cancer is glioma, a head and neck cancer, or prostate cancer. In embodiments, the cancer is glioma. In embodiments, the cancer is a head and neck cancer. In embodiments, the cancer is a head and neck squamous cell carcinoma. In embodiments, the cancer is prostate cancer.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus medulloblastoma, colorectal cancer, or pancreatic cancer. Additional examples include Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

In embodiments, the disclosure provides methods of delivering a lipid nanoparticle to a myeloid cell in a patient in need thereof by administering to the subject an effective amount of a lipid nanoparticle comprising a nucleic acid (e.g., RNA, mRNA, miRNA, siRNA, DNA, CpG ODN, CpG-ODN linked to RNA, CpG-ODN linked to siRNA) encapsulated therein. In embodiments, the disclosure provides methods of delivering a plurality of lipid nanoparticles to myeloid cells in a patient in need thereof by administering to the subject an effective amount of a plurality of lipid nanoparticles comprising a nucleic acid (e.g., RNA, mRNA, miRNA, siRNA, DNA, CpG ODN, CpG-ODN linked to siRNA) encapsulated therein. In embodiments, the disclosure provides methods of delivering lipid nanoparticles to myeloid cells in a subject in need thereof by administering to the subject an effective amount of a pharmaceutical compositions which comprise the lipid nanoparticles comprising a nucleic acid (e.g., RNA, mRNA, miRNA, siRNA, DNA, CpG ODN, CpG-ODN linked to siRNA) encapsulated therein, and a pharmaceutically acceptable excipient. In embodiments, the myeloid cells are lymph node myeloid cells. In embodiments, the nucleic acid is RNA or DNA. In embodiments, the nucleic acid is DNA. In embodiments, the nucleic acid is RNA. In embodiments, the RNA is mRNA, miRNA, or siRNA. In embodiments, the nucleic acid is mRNA. In embodiments, the nucleic acid is miRNA. In embodiments, the nucleic acid is siRNA. In embodiments, the nucleic acid is CpG-ODN linked to RNA. In embodiments, the nucleic acid is CpG-ODN linked to siRNA. In embodiments, the nucleic acid is CpG-ODN linked to miRNA. In embodiments, the nucleic acid comprises any one of SEQ ID NOS: 9-12. In embodiments, the nucleic acid comprises SEQ ID NO: 13 bonded to SEQ ID NO:9. In embodiments, the nucleic acid comprises SEQ ID NO:13 bonded to SEQ ID NO:9, wherein SEQ ID NO:9 is hybridized to SEQ ID NO: 10. In embodiments, the nucleic acid comprises SEQ ID NO: 13 bonded to SEQ ID NO:11. In embodiments, the nucleic acid comprises SEQ ID NO: 13 bonded to SEQ ID NO:11, wherein SEQ ID NO:11 is hybridized to SEQ ID NO: 12. In embodiments, the nucleic acid comprises SEQ ID NO:15 or 16. In embodiments, the nucleic acid comprises SEQ ID NO:15. In embodiments, the nucleic acid comprises SEQ ID NO: 15 hybridized to SEQ ID NO:10. In embodiments, the nucleic acid comprises SEQ ID NO:6. In embodiments, the nucleic acid comprises SEQ ID NO:6 hybridized to SEQ ID NO:12. In embodiments, the nucleic acid comprises SEQ ID NO: 16, SEQ ID NO:17, or SEQ ID NO: 16 hybridized to SEQ ID NO: 17. In embodiments, the nucleic acid comprises SEQ ID NO: 16. In embodiments, the nucleic acid comprises SEQ ID NO: 17. In embodiments, the nucleic acid comprises SEQ ID NO:16 hybridized to SEQ ID NO:17.

In embodiments of the methods of delivering to a myeloid cell, the myeloid cell is a monocyte, a dendritic cell, a macrophage, or a granulocyte. In embodiments, the myeloid cell is a monocyte. In embodiments, the myeloid cell is a dendritic cell. In embodiments, the myeloid cell is a macrophage. In embodiments, the myeloid cell is a granulocyte. In embodiments, the myeloid cell is within a tumor. In embodiments, the myeloid cell is within a cancer tumor. In embodiments, the myeloid cell is within a cancer tumor, wherein the cancer is lymphoma or leukemia. In embodiments, the myeloid cell is within a cancer tumor, wherein the cancer is B cell lymphoma, myeloid leukemia, glioma, head and neck cancer, or prostate cancer. In embodiments, the myeloid cell is within a cancer tumor, wherein the cancer is acute myeloid leukemia. In embodiments, the myeloid cell is within a cancer tumor, wherein the cancer is glioma, head and neck cancer, or prostate cancer. In embodiments, the myeloid cell is within a lymphoid organ. In embodiments, the monocyte is within a lymphoid organ. In embodiments, the dendritic cell is within a lymphoid organ. In embodiments, the macrophage is within a lymphoid organ. In embodiments, the granulocyte is within a lymphoid organ. In embodiments, the lymphoid organ is a lymph node, spleen, thymus, or bone marrow. In embodiments, the lymphoid organ is a spleen. In embodiments, the lymphoid organ is a thymus. In embodiments, the lymphoid organ is bone marrow. In embodiments, the lymphoid organ is red bone marrow. In embodiments, the lymphoid organ is a lymph node. In embodiments, the lymph node is a peripheral lymph node. In embodiments, the lymph node is a cervical lymph node, an axillary lymph node, or an inguinal lymph node. In embodiments, the lymph node is a cervical lymph node. In embodiments, the lymph node is an axillary lymph node. In embodiments, the lymph node is an inguinal lymph node.

In embodiments, the disclosure provides methods of delivering a lipid nanoparticle to a lymphoid organ in a patient in need thereof by administering to the subject an effective amount of a lipid nanoparticle comprising a nucleic acid (e.g., RNA, mRNA, miRNA, siRNA, DNA, CpG ODN, CpG-ODN linked to siRNA) encapsulated therein. In embodiments, the disclosure provides methods of deliverying a plurality of lipid nanoparticles to a lymphoid organ in a patient in need thereof by administering to the subject an effective amount of a plurality of lipid nanoparticles comprising a nucleic acid (e.g., RNA, mRNA, miRNA, siRNA, DNA, CpG ODN, CpG-ODN linked to siRNA) encapsulated therein. In embodiments, the disclosure provides methods of delivering lipid nanoparticles to a lymphoid organ in a subject in need thereof by administering to the subject an effective amount of a pharmaceutical compositions which comprise the lipid nanoparticles comprising a nucleic acid (e.g., RNA, mRNA, miRNA, siRNA, DNA, CpG ODN, CpG-ODN linked to siRNA) encapsulated therein, and a pharmaceutically acceptable excipient. In embodiments, the lymphoid organ is a lymph node, spleen, thymus, or bone marrow. In embodiments, the lymphoid organ is a spleen. In embodiments, the lymphoid organ is a thymus. In embodiments, the lymphoid organ is bone marrow. In embodiments, the lymphoid organ is red bone marrow. In embodiments, the lymphoid organ is a lymph node. In embodiments, the lymph node is a peripheral lymph node. In embodiments, the lymph node is a cervical lymph node, an axillary lymph node, or an inguinal lymph node. In embodiments, the lymph node is a cervical lymph node. In embodiments, the lymph node is an axillary lymph node. In embodiments, the lymph node is an inguinal lymph node. In embodiments, the nucleic acid is RNA or DNA. In embodiments, the nucleic acid is DNA. In embodiments, the nucleic acid is RNA. In embodiments, the RNA is mRNA, miRNA, or siRNA. In embodiments, the nucleic acid is mRNA. In embodiments, the nucleic acid is miRNA. In embodiments, the nucleic acid is siRNA. In embodiments, the nucleic acid is CpG-ODN linked to RNA. In embodiments, the nucleic acid is CpG-ODN linked to siRNA. In embodiments, the nucleic acid is CpG-ODN linked to miRNA. In embodiments, the nucleic acid comprises any one of SEQ ID NOS: 9-12. In embodiments, the nucleic acid comprises SEQ ID NO: 13 bonded to SEQ ID NO:9. In embodiments, the nucleic acid comprises SEQ ID NO: 13 bonded to SEQ ID NO:9, wherein SEQ ID NO:9 is hybridized to SEQ ID NO:10. In embodiments, the nucleic acid comprises SEQ ID NO: 13 bonded to SEQ ID NO:11. In embodiments, the nucleic acid comprises SEQ ID NO: 13 bonded to SEQ ID NO: 11, wherein SEQ ID NO: 11 is hybridized to SEQ ID NO: 12. In embodiments, the nucleic acid comprises SEQ ID NO:15 or 16. In embodiments, the nucleic acid comprises SEQ ID NO:15. In embodiments, the nucleic acid comprises SEQ ID NO:15 hybridized to SEQ ID NO:10. In embodiments, the nucleic acid comprises SEQ ID NO:6. In embodiments, the nucleic acid comprises SEQ ID NO:6 hybridized to SEQ ID NO: 12. In embodiments, the nucleic acid comprises SEQ ID NO:16, SEQ ID NO: 17, or SEQ ID NO:16 hybridized to SEQ ID NO:17. In embodiments, the nucleic acid comprises SEQ ID NO:16. In embodiments, the nucleic acid comprises SEQ ID NO:17. In embodiments, the nucleic acid comprises SEQ ID NO: 16 hybridized to SEQ ID NO:17.

In embodiments, the disclosure provides methods of delivering a lipid nanoparticle to a tumor in a patient in need thereof by administering to the subject an effective amount of a lipid nanoparticle comprising a nucleic acid (e.g., RNA, mRNA, miRNA, siRNA, DNA, CpG ODN, CpG-ODN linked to siRNA) encapsulated therein. In embodiments, the disclosure provides methods of deliverying a plurality of lipid nanoparticles to a tumor in a patient in need thereof by administering to the subject an effective amount of a plurality of lipid nanoparticles comprising a nucleic acid (e.g., RNA, mRNA, miRNA, siRNA, DNA, CpG ODN, CpG-ODN linked to siRNA) encapsulated therein. In embodiments, the disclosure provides methods of delivering lipid nanoparticles to a tumor in a subject in need thereof by administering to the subject an effective amount of a pharmaceutical compositions which comprise the lipid nanoparticles comprising a nucleic acid (e.g., RNA, mRNA, miRNA, siRNA, DNA, CpG ODN, CpG-ODN linked to siRNA) encapsulated therein, and a pharmaceutically acceptable excipient. In embodiments, the tumor is a cancer tumor. In embodiments, the tumor is a cancer tumor, wherein the cancer is a solid cancer. In embodiments, the cancer tumor is B cell lymphoma, myeloid leukemia, glioma, head and neck cancer, or prostate cancer. In embodiments, the cancer tumor is acute myeloid leukemia. In embodiments, the cancer tumor is glioma, head and neck cancer, or prostate cancer. In embodiments, the tumor is in a lymphoid organ. In embodiments, the tumor is in a lymph node, spleen, thymus, or bone marrow. In embodiments, the tumor is in a spleen. In embodiments, the tumor is in a thymus. In embodiments, the tumor is in bone marrow. In embodiments, the tumor is in is red bone marrow. In embodiments, the tumor is in is a lymph node. In embodiments, the nucleic acid is RNA or DNA. In embodiments, the nucleic acid is DNA. In embodiments, the nucleic acid is RNA. In embodiments, the RNA is mRNA, miRNA, or siRNA. In embodiments, the nucleic acid is mRNA. In embodiments, the nucleic acid is miRNA. In embodiments, the nucleic acid is siRNA. In embodiments, the nucleic acid is CpG-ODN linked to RNA. In embodiments, the nucleic acid is CpG-ODN linked to siRNA. In embodiments, the nucleic acid is CpG-ODN linked to miRNA. In embodiments, the nucleic acid comprises any one of SEQ ID NOS: 9-12. In embodiments, the nucleic acid comprises SEQ ID NO: 13 bonded to SEQ ID NO:9. In embodiments, the nucleic acid comprises SEQ ID NO:13 bonded to SEQ ID NO:9, wherein SEQ ID NO:9 is hybridized to SEQ ID NO:10. In embodiments, the nucleic acid comprises SEQ ID NO: 13 bonded to SEQ ID NO: 11. In embodiments, the nucleic acid comprises SEQ ID NO: 13 bonded to SEQ ID NO:11, wherein SEQ ID NO:11 is hybridized to SEQ ID NO:12. In embodiments, the nucleic acid comprises SEQ ID NO:15 or 16. In embodiments, the nucleic acid comprises SEQ ID NO: 15. In embodiments, the nucleic acid comprises SEQ ID NO:15 hybridized to SEQ ID NO:10. In embodiments, the nucleic acid comprises SEQ ID NO:6. In embodiments, the nucleic acid comprises SEQ ID NO:6 hybridized to SEQ ID NO: 12. In embodiments, the nucleic acid comprises SEQ ID NO: 16, SEQ ID NO:17, or SEQ ID NO: 16 hybridized to SEQ ID NO: 17. In embodiments, the nucleic acid comprises SEQ ID NO: 16. In embodiments, the nucleic acid comprises SEQ ID NO: 17. In embodiments, the nucleic acid comprises SEQ ID NO:16 hybridized to SEQ ID NO:17.

### Dose and Dosing Regimens

The dosage and frequency (single or multiple doses) of the nucleic acids (e.g., DNA, RNA, mRNA, miRNA, siRNA, DNA, CpG ODN, CpG-ODN linked to siRNA), lipid nanoparticles having nucleic acids encapsulated therein, or pharmaceutical compositions lipid nanoparticles having nucleic acids encapsulated therein administered to a subject can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and nucleic acids (e.g., DNA, RNA, mRNA, miRNA, siRNA, DNA, CpG ODN, CpG-ODN linked to siRNA), lipid nanoparticles having nucleic acids encapsulated therein, or pharmaceutical compositions lipid nanoparticles having nucleic acids encapsulated therein described herein. Adjustment and manipulation of established dosages (e.g., frequency and duration) are within the ability of the skilled artisan.

For any nucleic acid (e.g., DNA, RNA, mRNA, miRNA, siRNA, CpG ODN, CpG-ODN linked to siRNA), lipid nanoparticles having nucleic acids encapsulated therein, or pharmaceutical compositions lipid nanoparticles having nucleic acids encapsulated therein, as described herein, the effective amount can be initially determined from cell culture assays. Target concentrations will be those concentrations of nucleic acids (e.g., DNA, RNA, mRNA, miRNA, siRNA, DNA, CpG ODN, CpG-ODN linked to siRNA), lipid nanoparticles having nucleic acids encapsulated therein, or pharmaceutical compositions lipid nanoparticles having nucleic acids encapsulated therein that are capable of achieving the methods described herein, as measured using the methods described herein or known in the art. As is known in the art, effective amounts of nucleic acids (e.g., DNA, RNA, mRNA, miRNA, siRNA, DNA, CpG ODN, CpG-ODN linked to siRNA), lipid nanoparticles having nucleic acids encapsulated therein, or pharmaceutical compositions lipid nanoparticles having nucleic acids encapsulated therein for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the nucleic acids (e.g., DNA RNA, mRNA, miRNA, siRNA, DNA, CpG ODN, CpG-ODN linked to siRNA), lipid nanoparticles having nucleic acids encapsulated therein, or pharmaceutical compositions lipid nanoparticles having nucleic acids encapsulated therein may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Dosage amounts and intervals can be adjusted individually to provide levels of the nucleic acids (e.g., DNA, RNA, mRNA, miRNA, siRNA, DNA, CpG ODN, CpG-ODN linked to siRNA), lipid nanoparticles having nucleic acids encapsulated therein, or pharmaceutical compositions lipid nanoparticles having nucleic acids encapsulated therein effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical disease or symptoms demonstrated by the particular patient. This planning should involve the careful choice of nucleic acids (e.g., DNA, RNA, sense RNA, antisense RNA, siRNA mRNA, miRNA, DNA, plasmid, minigene), lipid nanoparticles having nucleic acids encapsulated therein, or pharmaceutical compositions lipid nanoparticles having nucleic acids encapsulated therein by considering factors such as compound potency, relative bioavailability, patient body weight, presence and severity of adverse side effects.

In embodiments, the nucleic acid (e.g., DNA, RNA, mRNA, miRNA, siRNA, DNA, CpG ODN, CpG-ODN linked to siRNA), lipid nanoparticles having nucleic acids encapsulated therein, or pharmaceutical compositions lipid nanoparticles having nucleic acids encapsulated therein is administered to a patient at an amount of about 0.001 mg/kg to about 500 mg/kg. In embodiments, the nucleic acids is administered to a patient in an amount of about 0.01 mg/kg, 0.1 mg/kg, 0.5 mg/kg, 1 mg/kg, 2 mg/kg, 3 mg/kg, 4 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, or 300 mg/kg. It is understood that where the amount is referred to as “mg/kg,” the amount is milligram per kilogram body weight of the subject being administered with the nucleic acid. In embodiments, the nucleic acid is administered to a patient in an amount from about 0.001 mg to about 500 mg per day, as a single dose, or in a dose administered two or three times per day.

### Embodiments 1-96

Embodiment 1. A lipid nanoparticle comprising about: (i) 40 mole % to about 60 mole % of a cationic lipid; (ii) about 5 mole % to about 15 mole % of a phospholipid; (iii) about 28 mole % to 50 mole % of a sterol; and (iv) about 0.1 mole % to about 4 mole % of a polyethylene glycol-lipid conjugate.

Embodiment 2. The lipid nanoparticle of Embodiment 1 comprising: (i) about 48 mole % to about 52 mole % of the cationic lipid; (ii) about 8 mole % to about 12 mole % of the phospholipid; (iii) about 35 mole % to 42 mole % of the sterol; and (iv) about 0.1 mole % to about 2 mole % of the polyethylene glycol-lipid conjugate.

Embodiment 3. The lipid nanoparticle of Embodiment 1 comprising: (i) about 50 mole % of the cationic lipid; (ii) about 10 mole % of the phospholipid; (iii) about 38.5 mole % of the sterol; and (iv) about 1.5 mole % of the polyethylene glycol-lipid conjugate.

Embodiment 4. The lipid nanoparticle of Embodiment 1 comprising: (i) about 50 mole % of the cationic lipid; (ii) about 10 mole % of the phospholipid; (iii) about 39.5 mole % of the sterol; and (iv) about 0.5 mole % of the polyethylene glycol-lipid conjugate.

Embodiment 5. The lipid nanoparticle of any one of Embodiments 1 to 4, wherein the cationic lipid is a dilinoleic cationic lipid.

Embodiment 6. The lipid nanoparticle of Embodiment 5, wherein the dilinoleic cationic lipid is MC3, an MC3 derivative, DLinDMA, DLin-K-C2-DMA, DLin-K-C3-DMA, DLin-K-C4-DMA, DLin-K6-DMA, DLin-K-MPZ, DLin-K-DMA, DLin-C-DAP, DLin-DAC, DLin-MA, DLinDAP, DLin-S-DMA, DLin-2-DMAP, DLin-TMA, DLin-TAP, DLin-MPZ, DLinAP, DLin-EG-DMA, DLincarbDAP, or a mixture of two or more thereof.

Embodiment 7. The lipid nanoparticle of Embodiment 5, wherein the dilinoleic cationic lipid is MC3.

Embodiment 8. The lipid nanoparticle of any one of Embodiments 1 to 7, wherein the phospholipid is DSPC, DPPC, DOPE, POPC, POPE, POPG, DPPE, DMPE, DSPE, MMPE, DMPE, DEPE, SOPE, EPC, HSPC, or a mixture of two or more thereof.

Embodiment 9. The lipid nanoparticle of any one of Embodiments 1 to 7, wherein the phospholipid is DSPC, DPPC, DOPE, POPC, POPE, POPG, DPPE, DMPE, DSPE, MMPE, DMPE, DEPE, SOPE, EPC, HSPC, DPPG, or a mixture of two or more thereof.

Embodiment 10. The lipid nanoparticle of Embodiment 8 or 9, wherein the phospholipid is HSPC.

Embodiment 11. The lipid nanoparticle of Embodiment 9, wherein the phospholipid is DPPG.

Embodiment 12. The lipid nanoparticle of any one of Embodiments 1 to 11, wherein the sterol is cholesterol, cholestanol, cholestanone, cholestenone, coprostanol, cholesteryl-2′-hydroxyethyl ether, cholesteryl-4′-hydroxybutyl ether, or a mixture of two or more thereof.

Embodiment 13. The lipid nanoparticle of any one of Embodiments 1 to 11, wherein the sterol is cholesterol, cholestanol, cholestanone, cholestenone, coprostanol, cholesteryl-2′-hydroxyethyl ether, cholesteryl-4′-hydroxybutyl ether, cholesteryl hemisuccinate, or a mixture of two or more thereof.

Embodiment 14. The lipid nanoparticle of Embodiment 12 or 13, wherein the sterol is cholesterol.

Embodiment 15. The lipid nanoparticle of Embodiment 13, wherein the sterol is a mixture of cholesterol and cholesteryl hemisuccinate.

Embodiment 16. The lipid nanoparticle of any one of Embodiments 1 to 15, wherein the polyethylene glycol-lipid conjugate is a polyethylene glycol having a molecular weight from about 1,000 Daltons to about 6,000 Daltons conjugated to a C₁₂-C₂₂ fatty acid lipid.

Embodiment 17. The lipid nanoparticle of Embodiment 16, wherein the polyethylene glycol has a molecular weight of about 2,000 Daltons.

Embodiment 18. The lipid nanoparticle of Embodiment 16 or 17, wherein the polyethylene glycol-lipid conjugate is DMG-PEG, DPPE-PEG, DPG-PEG, DSG-PEG, DSPE-PEG, or a mixture of two or more thereof.

Embodiment 19. The lipid nanoparticle of Embodiment 16 or 17, wherein the polyethylene glycol-lipid conjugate is DMG-PEG.

Embodiment 20. The lipid nanoparticle of any one of Embodiments 1 to 4, wherein (i) the cationic lipid is MC3; (ii) the phospholipid is HSPC; (iii) the sterol is cholesterol; and (iv) the polyethylene glycol-conjugated lipid is DMG-PEG, wherein the polyethylene glycol has a molecular weight of about 2,000 Daltons.

Embodiment 21. The lipid nanoparticle of any one of Embodiments 1 to 4, wherein (i) the cationic lipid is MC3; (ii) the phospholipid is DPPG; (iii) the sterol is cholesterol; and (iv) the polyethylene glycol-conjugated lipid is DMG-PEG, wherein the polyethylene glycol has a molecular weight of about 2,000 Daltons.

Embodiment 22. The lipid nanoparticle of any one of Embodiments 1 to 4, wherein (i) the cationic lipid is MC3; (ii) the phospholipid is HSPC; (iii) the sterol is cholesterol and cholesteryl hemisuccinate; and (iv) the polyethylene glycol-conjugated lipid is DMG-PEG, wherein the polyethylene glycol has a molecular weight of about 2,000 Daltons.

Embodiment 23. The lipid nanoparticle of Embodiment 22, wherein the molar ratio of cholesterol to cholesteryl hemisuccinate is 2:1 to 1:2.

Embodiment 24. The lipid nanoparticle of Embodiment 22, wherein the sterol is 18.5 mole % cholesterol and 20 mol % cholesteryl hemisuccinate.

Embodiment 25. The lipid nanoparticle of any one of Embodiments 1 to 24, wherein a plurality of the lipid nanoparticles has an average size from about 50 nm to about 150 nm.

Embodiment 26. The lipid nanoparticle of any one of Embodiments 1 to 25, wherein a plurality of the lipid nanoparticles has a zeta potential from about −5 mV to about −35 mV.

Embodiment 27. The lipid nanoparticle of any one of Embodiments 1 to 25, wherein a plurality of the lipid nanoparticles has a zeta potential from about −5 mV to about −20 mV.

Embodiment 28. The lipid nanoparticle of any one of Embodiments 1 to 27, further comprising a nucleic acid encapsulated within the lipid nanoparticle.

Embodiment 29. The lipid nanoparticle of Embodiment 28, wherein the nucleic acid is DNA, RNA, or a combination thereof.

Embodiment 30. The lipid nanoparticle of Embodiment 28, wherein the nucleic acid is RNA.

Embodiment 31. The lipid nanoparticle of Embodiment 28, wherein the nucleic acid is siRNA.

Embodiment 32. The lipid nanoparticle of Embodiment 28, wherein the nucleic acid is STAT3 siRNA.

Embodiment 33. The lipid nanoparticle of Embodiment 28, wherein the nucleic acid comprises SEQ ID NO:7.

Embodiment 34. The lipid nanoparticle of Embodiment 33, wherein SEQ ID NO:7 is hybridized to SEQ ID NO:8.

Embodiment 35. The lipid nanoparticle of Embodiment 28, wherein the nucleic acid comprises SEQ ID NO:7, SEQ ID NO:8, SEQ ID NO:9, SEQ ID NO: 10, SEQ ID NO:11, SEQ ID NO: 12, or SEQ ID NO:13.

Embodiment 36. The lipid nanoparticle of Embodiment 28, wherein the nucleic acid comprises SEQ ID NO: 7 hybridized to SEQ ID NO:8; SEQ ID NO:9 hybridized to SEQ ID NO: 10; or SEQ ID NO: 11 hybridized to SEQ ID NO:12.

Embodiment 37. The lipid nanoparticle of Embodiment 28, wherein the nucleic acid is a CpG ODN attached to an antisense strand of STAT3 siRNA.

Embodiment 38. The lipid nanoparticle of Embodiment 37, wherein the antisense strand of STAT3 siRNA is hybridized to a complementary sense strand of siRNA.

Embodiment 39. The lipid nanoparticle of Embodiment 28, wherein the nucleic acid is a CpG ODN attached to a sense strand of STAT3 siRNA.

Embodiment 40. The lipid nanoparticle of Embodiment 39, wherein the sense strand of STAT3 siRNA is hybridized to a complementary antisense strand of siRNA.

Embodiment 41. The lipid nanoparticle of Embodiment 28, wherein the nucleic acid is SEQ ID NO:14.

Embodiment 42. The lipid nanoparticle of Embodiment 41, wherein SEQ ID NO:14 is hybridized to SEQ ID NO:8.

Embodiment 43. The lipid nanoparticle of Embodiment 28, wherein the nucleic acid is SEQ ID NO:15.

Embodiment 44. The lipid nanoparticle of Embodiment 43, wherein SEQ ID NO: 15 is hybridized to SEQ ID NO:10.

Embodiment 45. The lipid nanoparticle of Embodiment 28, wherein the nucleic acid is SEQ ID NO:6.

Embodiment 46. The lipid nanoparticle of Embodiment 45, wherein SEQ ID NO:6 is hybridized to SEQ ID NO:12.

Embodiment 47. The lipid nanoparticle of Embodiment 28, wherein the nucleic acid is miRNA.

Embodiment 48. The lipid nanoparticle of Embodiment 47, wherein the miRNA comprises SEQ ID NO: 16 or SEQ ID NO:17.

Embodiment 49. The lipid nanoparticle of Embodiment 47, wherein the miRNA comprises SEQ ID NO: 16 hybridized to SEQ ID NO:17.

Embodiment 50. The lipid nanoparticle of Embodiment 28, wherein the nucleic acid is mRNA.

Embodiment 51. The lipid nanoparticle of any one of Embodiments 28 to 50, wherein the N/P ratio is from about 2:1 to about 8:1.

Embodiment 52. The lipid nanoparticle of Embodiment 51, wherein the N/P ratio is about 3:1.

Embodiment 53. The lipid nanoparticle of Embodiment 51, wherein the N/P ratio is about 4:1 or about 5:1.

Embodiment 54. The lipid nanoparticle of Embodiment 51, wherein the N/P ratio is about 6:1.

Embodiment 55. A pharmaceutical composition comprising the lipid nanoparticle of any one of Embodiments 1 to 54 and a pharmaceutically acceptable excipient.

Embodiment 56. A method for treating cancer in a patient in need thereof, the method comprising administering to the patient the lipid nanoparticle of any one of Embodiments 1 to 54 or the pharmaceutical composition of Embodiment 55.

Embodiment 57. The method of Embodiment 56, wherein the cancer is lymphoma or leukemia.

Embodiment 58. The method of Embodiment 56, wherein the cancer is B cell lymphoma, myeloid leukemia, glioma, head and neck cancer, or prostate cancer.

Embodiment 59. The method of Embodiment 56, wherein the cancer is acute myeloid leukemia.

Embodiment 60. The method of any one of Embodiments 56 to 59, comprising parenterally administering to the patient the lipid nanoparticle, the plurality of the lipid nanoparticles, or the pharmaceutical composition.

Embodiment 61. The method of Embodiment 60, wherein parenterally administering is intravenously administering, subcutaneously administering, or intranodally administering.

Embodiment 62. A method of delivering a lipid nanoparticle to a myeloid cell in a patient in need thereof, the method comprising administering to the patient the lipid nanoparticle of any one of Embodiments 1 to 54 or the pharmaceutical composition of Embodiment 55.

Embodiment 63. The method of Embodiment 62, wherein the myeloid cell is a monocyte, a dendritic cell, a macrophage, or a granulocyte.

Embodiment 64. The method of Embodiment 62 or 63, wherein the myeloid cell is within a tumor.

Embodiment 65. The method of Embodiment 62 or 63, wherein the myeloid cell is within a lymphoid organ.

Embodiment 66. A method of delivering a lipid nanoparticle to a lymphoid organ in a patient in need thereof, the method comprising administering to the patient the lipid nanoparticle of any one of Embodiments 1 to 54 or the pharmaceutical composition of Embodiment 55.

Embodiment 67. The method of Embodiment 65 or 66, wherein the lymphoid organ is a lymph node, spleen, thymus, or bone marrow.

Embodiment 68. The method of Embodiment 67, wherein the lymphoid organ is the spleen.

Embodiment 69. The method of Embodiment 67, wherein the lymphoid organ is bone marrow.

Embodiment 70. The method of Embodiment 67, wherein the lymphoid organ is the lymph node.

Embodiment 71. The method of Embodiment 70, wherein the lymph node is a peripheral lymph node.

Embodiment 72. A method of delivering a lipid nanoparticle to a tumor in a patient in need thereof, the method comprising administering to the patient the lipid nanoparticle of any one of Embodiments 1 to 54 or the pharmaceutical composition of Embodiment 55.

Embodiment 73. A nucleic acid comprising a CpG ODN and SEQ ID NO:9.

Embodiment 74. The nucleic acid of Embodiment 73, wherein the CpG ODN is bonded to SEQ ID NO:9 via a substituted or unsubstituted 6 to 60 membered heteroalkylene.

Embodiment 75. The nucleic acid of Embodiment 73 or 74, wherein SEQ ID NO:9 is hybridized to SEQ ID NO:10.

Embodiment 76. A nucleic acid comprising a CpG ODN and SEQ ID NO:11.

Embodiment 77. The nucleic acid of Embodiment 76, wherein the CpG ODN is bonded to SEQ ID NO: 11 via a substituted or unsubstituted 6 to 60 membered heteroalkylene.

Embodiment 78. The nucleic acid of Embodiment 76 or 77, wherein SEQ ID NO:11 is hybridized to SEQ ID NO:12.

Embodiment 79. The nucleic acid of any one of Embodiments 73 to 78, wherein the CpG ODN comprises SEQ ID NO: 13.

Embodiment 80. A nucleic acid comprising SEQ ID NO: 15.

Embodiment 81. The nucleic acid of Embodiment 80, wherein SEQ ID NO: 15 is hybridized to SEQ ID NO:10.

Embodiment 82. A nucleic acid comprising SEQ ID NO:6.

Embodiment 83. The nucleic acid of Embodiment 82, wherein SEQ ID NO:6 is hybridized to SEQ ID NO:12.

Embodiment 84. A nucleic acid comprising SEQ ID NO:9, SEQ ID NO: 10, SEQ ID NO: 11, or SEQ ID NO: 12.

Embodiment 85. A nucleic acid comprising SEQ ID NO:7.

Embodiment 86. The nucleic acid of Embodiment 85, wherein SEQ ID NO: 7 is hybridized to SEQ ID NO:8.

Embodiment 87. A nucleic acid comprising SEQ ID NO: 14.

Embodiment 88. The nucleic acid of Embodiment 87, wherein SEQ ID NO: 14 is hybridized to SEQ ID NO:8.

Embodiment 89. The nucleic acid of any one of Embodiments 85 to 88, wherein the nucleic acid is bonded to a CpG ODN.

Embodiment 90. A pharmaceutical composition comprising the nucleic acid of any one of Embodiments 73 to 89.

Embodiment 91. A method for treating cancer in a patient in need thereof, the method comprising administering to the patient the nucleic acid of any one of Embodiments 73 to 89 or the pharmaceutical composition of Embodiment 90.

Embodiment 92. The method of Embodiment 91, wherein the cancer is lymphoma or leukemia.

Embodiment 93. The method of Embodiment 91, wherein the cancer is B cell lymphoma, myeloid leukemia, glioma, head and neck cancer, or prostate cancer.

Embodiment 94. The method of Embodiment 91, wherein the cancer is acute myeloid leukemia.

Embodiment 95. The method of any one of Embodiments 91 to 94, comprising parenterally administering the nucleic acid to the patient.

Embodiment 96. The method of Embodiment 95, wherein parenterally administering is intravenously administering, subcutaneously administering, or intranodally administering.

### Embodiments A1 to A29

Embodiment A1. A lipid nanoparticle comprising: (i) about 40 mole % to about 60 mole % of a cationic lipid; (ii) about 5 mole % to about 15 mole % of a phospholipid; (iii) about 28 mole % to 50 mole % of a sterol; and (iv) about 0.1 mole % to about 4 mole % of a polyethylene glycol-lipid conjugate

Embodiment A2. The lipid nanoparticle of Embodiment A1 comprising: (i) about 48 mole % to about 52 mole % of the cationic lipid; (ii) about 8 mole % to about 12 mole % of the phospholipid; (iii) about 35 mole % to 42 mole % of the sterol; and (iv) about 0.1 mole % to about 2 mole % of the polyethylene glycol-lipid conjugate

Embodiment A3. The lipid nanoparticle of Embodiment A1 comprising: (i) about 50 mole % of the cationic lipid; (ii) about 10 mole % of the phospholipid; (iii) about 38.5 mole % of the sterol; and (iv) about 1.5 mole % of the polyethylene glycol-lipid conjugate

Embodiment A4. The lipid nanoparticle of Embodiment A1 comprising: (i) about 50 mole % of the cationic lipid; (ii) about 10 mole % of the phospholipid; (iii) about 39.5 mole % of the sterol; and (iv) about 0.5 mole % of the polyethylene glycol-lipid conjugate

Embodiment A5. The lipid nanoparticle of any one of Embodiments A1 to A4, wherein (i) the cationic lipid is a dilinoleic cationic lipid; and wherein the dilinoleic cationic lipid is MC3, an MC3 derivative, DLinDMA, DLin-K-C2-DMA, DLin-K-C3-DMA, DLin-K-C4-DMA, DLin-K6-DMA, DLin-K-MPZ, DLin-K-DMA, DLin-C-DAP, DLin-DAC, DLin-MA, DLinDAP, DLin-S-DMA, DLin-2-DMAP, DLin-TMA, DLin-TAP, DLin-MPZ, DLinAP, DLin-EG-DMA, DLincarbDAP, or a mixture of two or more thereof; (ii) the phospholipid is DSPC, DPPC, DOPE, POPC, POPE, POPG, DPPE, DMPE, DSPE, MMPE, DMPE, DEPE, SOPE, EPC, HSPC, DPPG, or a mixture of two or more thereof; (iii) the sterol is cholesterol, cholestanol, cholestanone, cholestenone, coprostanol, cholesteryl-2′-hydroxyethyl ether, cholesteryl-4′-hydroxybutyl ether, cholesteryl hemisuccinate, or a mixture of two or more thereof; and (iv) the polyethylene glycol-lipid conjugate is a polyethylene glycol having a molecular weight from about 1,000 Daltons to about 6,000 Daltons conjugated to a C₁₂-C₂₂ fatty acid lipid

Embodiment A6. The lipid nanoparticle of Embodiment A5, wherein the dilinoleic cationic lipid is MC3.

Embodiment A7. The lipid nanoparticle of Embodiment A5 or A6, wherein the phospholipid is HSPC or DPPG.

Embodiment A8. The lipid nanoparticle of any one of Embodiments A5 to 7A, wherein the sterol is cholesterol.

Embodiment A9. The lipid nanoparticle of any one of Embodiments A5 to A7, wherein the sterol is a mixture of cholesterol and cholesteryl hemisuccinate.

Embodiment A10. The lipid nanoparticle of any one of Embodiments A5 to A9, wherein the polyethylene glycol has a molecular weight of about 2,000 Daltons.

Embodiment A11. The lipid nanoparticle of any one of Embodiments A5 to A10, wherein the polyethylene glycol-lipid conjugate is DMG-PEG, DPPE-PEG, DPG-PEG, DSG-PEG, DSPE-PEG, or a mixture of two or more thereof.

Embodiment A12. The lipid nanoparticle of Embodiment A11, wherein the polyethylene glycol-lipid conjugate is DMG-PEG.

Embodiment A13. The lipid nanoparticle of any one of Embodiments A1 to A4, wherein: (i) the cationic lipid is MC3; (iii) the phospholipid is HSPC; (iv) the sterol is cholesterol; and (v) the polyethylene glycol-conjugated lipid is DMG-PEG, wherein the polyethylene glycol has a molecular weight of about 2,000 Daltons

Embodiment A14. The lipid nanoparticle of any one of Embodiments A1 to A4, wherein: (i) the cationic lipid is MC3; (iii) the phospholipid is DPPG; (iv) the sterol is cholesterol; and (v) the polyethylene glycol-conjugated lipid is DMG-PEG, wherein the polyethylene glycol has a molecular weight of about 2,000 Daltons

Embodiment A15. The lipid nanoparticle of any one of Embodiments A1 to A4, wherein: (i) the cationic lipid is MC3; (iii) the phospholipid is HSPC; (iv) the sterol is cholesterol and cholesteryl hemisuccinate; and (v) the polyethylene glycol-conjugated lipid is DMG-PEG, wherein the polyethylene glycol has a molecular weight of about 2,000 Daltons

Embodiment A16. The lipid nanoparticle of Embodiment A15, wherein the molar ratio of cholesterol to cholesteryl hemisuccinate is 2:1 to 1:2.

Embodiment A17. The lipid nanoparticle of Embodiment A15, wherein the sterol is 18.5 mole % cholesterol and 20 mol % cholesteryl hemisuccinate.

Embodiment A18. The lipid nanoparticle of any one of Embodiments A1 to A17, wherein a plurality of the lipid nanoparticles have an average size from about 50 nm to about 150 nm.

Embodiment A19. The lipid nanoparticle of any one of Embodiments A1 to A18, further comprising a nucleic acid encapsulated within the lipid nanoparticle.

Embodiment A20. The lipid nanoparticle of Embodiment A19, wherein the nucleic acid is DNA or RNA.

Embodiment A21. The lipid nanoparticle of Embodiment A19, wherein the nucleic acid is siRNA, miRNA, or mRNA.

Embodiment A22. The lipid nanoparticle of Embodiment A19, wherein the nucleic acid is STAT3 siRNA.

Embodiment A23. The lipid nanoparticle of Embodiment A19, wherein the nucleic acid comprises SEQ ID NO:7; SEQ ID NO:7 hybridized to SEQ ID NO:8; SEQ ID NO:8; SEQ ID NO: 9; SEQ ID NO: 10; SEQ ID NO: 11; SEQ ID NO: 12; SEQ ID NO: 13; SEQ ID NO:7 hybridized to SEQ ID NO:8; SEQ ID NO:9 hybridized to SEQ ID NO: 10; SEQ ID NO: 11 hybridized to SEQ ID NO:12; SEQ ID NO:14; SEQ ID NO: 14 hybridized to SEQ ID NO:8; SEQ ID NO:15; SEQ ID NO:15 hybridized to SEQ ID NO:10; SEQ ID NO:6; SEQ ID NO:6 hybridized to SEQ ID NO:12; SEQ ID NO:16; SEQ ID NO: 17; or SEQ ID NO: 16 hybridized to SEQ ID NO:17.

Embodiment A24. The lipid nanoparticle of Embodiment A19, wherein the nucleic acid is: (i) a CpG ODN attached to an antisense strand of STAT3 siRNA; (ii) a CpG ODN attached to an antisense strand of STAT3 siRNA, wherein the antisense strand of STAT3 siRNA is hybridized to a complementary sense strand of siRNA; (iii) a CpG ODN attached to a sense strand of STAT3 siRNA; or (iv) a CpG ODN attached to a sense strand of STAT3 siRNA, wherein the sense strand of STAT3 siRNA is hybridized to a complementary antisense strand of siRNA.

Embodiment A25. The lipid nanoparticle of any one of Embodiments A19 to A24, wherein the N/P ratio is from about 2:1 to about 8:1.

Embodiment A26. A pharmaceutical composition comprising the lipid nanoparticle of any one of Embodiments A1 to A25 and a pharmaceutically acceptable excipient.

Embodiment A27. A method for treating cancer in a patient in need thereof, the method comprising administering to the patient the lipid nanoparticle of any one of Embodiments A1 to A25 or the pharmaceutical composition of Embodiment A26.

Embodiment A28. A method of delivering a lipid nanoparticle to a myeloid cell, a lymphoid organ, or a tumor in a patient in need thereof, the method comprising administering to the patient the lipid nanoparticle of any one of Embodiments A1 to A25 or the pharmaceutical composition of Embodiment A26.

Embodiment A29. A nucleic acid comprising: (i) a CpG ODN bonded to SEQ ID NO:9 via a substituted or unsubstituted 6 to 60 membered heteroalkylene; (ii) SEQ ID NO:9 is hybridized to SEQ ID NO:10; (iii) a CpG ODN is bonded to SEQ ID NO: 11 via a substituted or unsubstituted 6 to 60 membered heteroalkylene; (iv) SEQ ID NO:11 is hybridized to SEQ ID NO: 12; (v) SEQ ID NO:15; (vi) SEQ ID NO:15 is hybridized to SEQ ID NO:10; (vii) SEQ ID NO: 6; (viii) SEQ ID NO:6 is hybridized to SEQ ID NO:12; (ix) SEQ ID NO:9; (x) SEQ ID NO: 10; (xi) SEQ ID NO: 11; (xii) SEQ ID NO: 12: (xiii) SEQ ID NO:7; (xiv) SEQ ID NO:7 is hybridized to SEQ ID NO:8; (xv) SEQ ID NO:14; or (xvi) SEQ ID NO:14 is hybridized to SEQ ID NO: 8.

### EXAMPLES

### Example 1

Lipid nanoparticles having the components shown in Table 5 were prepared and characterized by methods known in the art. Lipid nanoparticle formulations containing the CpG ODN-STAT3 siRNA encapsulated therein are shown in Table 6.

TABLE 5

| Formulation | | | | |
|---|---|---|---|---|
| Name | MC3 | HSPC | Cholesterol | DMG-PEG2000 |
| Formulation 1 | 50 | 10 | 38.5 | 1.5 |
| Formulation 2 | 50 | 10 | 39.5 | 0.5 |

TABLE 6

| Formulation | | | | DMG- | | Nucleic |
|---|---|---|---|---|---|---|
| Name | MC3 | HSPC | Cholesterol | PEG2000 | N/P | Acid |
| LNP-1 | 50 | 10 | 38.5 | 1.5 | 3 | CSI-1A |
| LNP-2 | 50 | 10 | 38.5 | 1.5 | 6 | CSI-1A |
| LNP-3 | 50 | 10 | 39.5 | 0.5 | 3 | CSI-1A |
| LNP-4 | 50 | 10 | 39.5 | 0.5 | 6 | CSI-1A |
| A | 50 | 10 | 38.5 | 1.5 | 6 | #3.3 |
| B | 50 | 10 | 38.5 | 1.5 | 6 | #3.4 |

Reagents needed: 1×sterile PBS, ethanol (absolute), pre-aliquoted MC-3 dissolved in ethanol (10 mg/ml), citrate buffer (50 mM), ultrafiltration tubes (Ultra-15), 1 ml syringe and 3 ml syringe, and 15 ml conical tubes

The lipids were dissolved in ethanol as 10 mg/ml. The lipid mixture was prepared using pre-calculated volume. The oligo aqueous phase was prepared using citrate buffer (50 mM) depends on the N/P ratio (i.e., 0.2 mg for N/P=3 and 0.1 mg for N/P=6). After preparation, the microfluidic chip was primed with exact same setting/volume of ethanol and citrate buffer. The aqueous phase (containing oligonucleotides) was loaded onto the left inlay of Nanoassmblr and the organic phase (lipid mix) was loaded onto the right inlay of Nanoassmblr. For the NanoAssmblr program, the total flow rate was 9 ml/min; flow rate ratio is 3:1 and 0.2 ml start waste, 0.05 ml end waste. After the lipid nanoparticle was produced (in 15 ml conical collection tube), it was diluted with 1×PBS immediately up to 15 ml. The lipid nanoparticles were purified (from excess ethanol and oligo) using Ultra-15 centrifuge tubes at 2000×g for 30 min; the flow through was discarded, and then the lipid nanoparticles were diluted up to 15 ml again, and centrifuged at 2000×g for 25-30 min depends on desired final volume. After the final centrifugation, the product was diluted to original designed volume (about 2.5 mg lipid/ml) and push through 0.22 μm filter and store at 4° C.

Standard Characterization Procedure are described below.

Loading/Encapsulation efficiency. Purified LNPs and 4% Triton-X was incubated at 37° C. for about 20 min, released nucleic acid content was determined through Quant it Ribogreen Assay Kit (R11490) using original nucleic acid as standard. Final readout was measured using Cytation3 with fluorescent measurement at Ex/Em 485/528 nm. The final nucleic acid concentration was kept under 100 ng/ml with proper dilutions.

Zeta potential to determine surface charge of CpG-siSTAT3 encapsulated LNPs was measured by 1:1000 dilution of LNPs with 10 mM NaCl and performing 10 runs at 30 cycles per run.

Transmission electron microscopy: 1:10 diluted LNPs (1×PBS) were absorbed to carbon coated 200 mesh EM grids. Samples were prepared by conventional negative staining with 2% (w/v) uranyl acetate and EM images were obtained on PEI Tecnai 12 transmission electron microscope equipped with Gatan OneView CMOS camera.

Nanoparticle Tracking Analysis: size and concentration of LNPs were measured using Nanosight (Malvern NS300). Samples were first diluted with PBS (300×) and measurements were obtained by performing 3 runs of 60 sec, flow rate (30).

### Example 2

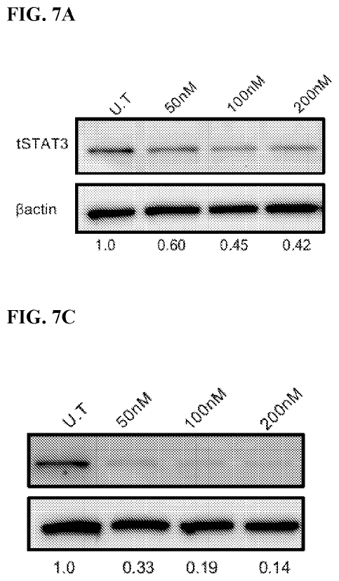

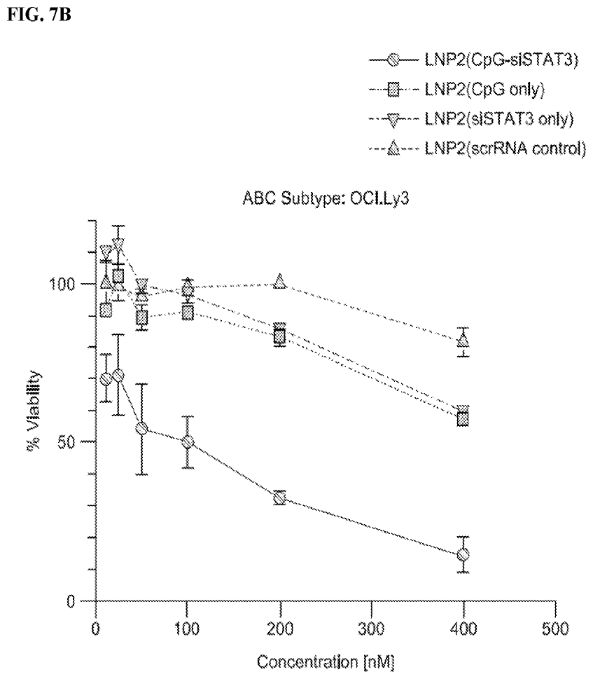

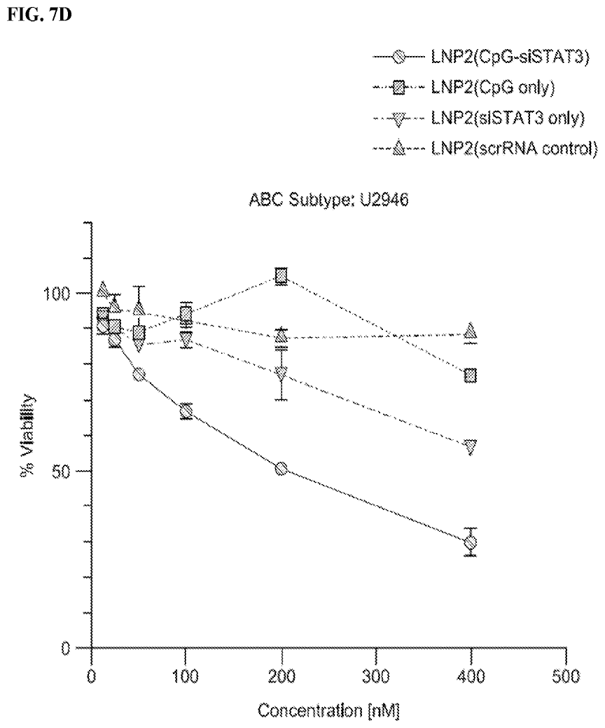

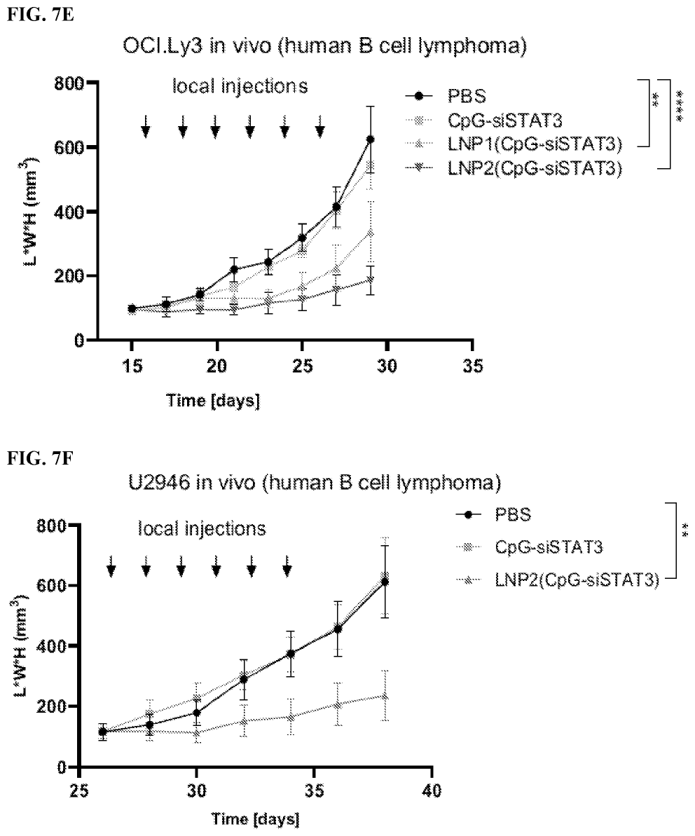

Recent reports showcased the potential to target oligonucleotides to certain organs through the incorporation of various charged helper lipid (Cheng et al, Nat Nanotechnol, 15(4): 313-320 (2020); Dilliard et al, PNAS USA, 118(52) (2021)). The anionic lipid incorporation into LNPs facilitated the delivery of encapsulated cargo such as mRNA to the spleen (LoPresti et al. Journal of Controlled Release, 345:819-831 (2022)). The studies described herein (e.g., FIGS. 7-10) focused on the generation and selection of new LNP formulations shown in Table 7, were designed to target not only lymphoid organs, such as spleen or lymph nodes, but also specific immune cell subsets within these locations. These LNP are myeloid cell-selective to deliver various DNA or RNA therapeutics to dendritic cells, macrophages or other myeloid cells, including acute myeloid leukemia. This approach is a new tool for cancer immunotherapy or for alleviating pathogenic immune activation.

Lipid nanoparticles having the components shown in Table 7 were prepared and characterized by methods known in the art. LNP2^{DPPG} is alternatively referred to as LNP2^{PG} or LNP2^{PPG}, where there is an optional dash between “P” and “2.”

TABLE 7

| Formulation | | | | | Cholesteryl | |
|---|---|---|---|---|---|---|
| Name | MC3 | HSPC | DPPG | Cholesterol | Hemisuccinate | DMG-PEG2000 |
| LNP2^{DPPG} | 50 | 0 | 10 | 38.5 | 0 | 1.5 |
| LNP2^{CHEM} | 50 | 10 | 0 | 18.5 | 20 | 1.5 |

With reference to FIGS. 7A-7D: Western blot analyses (left panels): 2×10⁵ OCI.Ly3 or U2946 cells were plated in 6-well culture plate and indicated treatments of 50, 100, 200 nM of LNP2 encapsulated CpG-STAT3siRNA were added to culture medium. Treated cells were collected after 72 h of incubation at 37° C. with 5% CO₂. Treated cells were lysed using RIPA buffer to analyze protein levels of total STAT3 and β-actin as loading control. Cell viability assessment (right panels): 2×10⁴ OCI.Ly3 or U2946 cells were plated in 96-well culture plate and indicated treatment using LNP2 loaded with CpG-STAT3siRNA, CpG ODN only, STAT3siRNA only, or scrambled RNA control added to culture medium at various dosing (referring to oligonucleotide concentrations). Treated cells viability were analyzed using a colorimetric dye relative to untreated cells. Shown are representative images and analyzed graphs of two independently repeated experiments (n=3) for MTT assay; means±SD

With reference to FIGS. 7E-7F: In vivo local administration of LNP2 loaded with CpG-STAT3siRNA results in tumor growth arrest for two different ABC-subtype of human B cell lymphoma. 5×10⁶ cells (OCI.Ly3 or U2946) were subcutaneously implanted to NSG (8-12 weeks old). After tumor volume exceeded 100 mm³ (L×W×H), mice were treated using local administration of 100 μl of the indicated treatments (PBS, CpG-STAT3siRNA, LNP1-encapsulated CpG-STAT3siRNA or LNP2-encapsulated CpG-STAT3siRNA) given every other day. Tumor volume was monitored using caliper measurements every other day until 1000 mm³. Shown are representative tumor growth curves of two independently repeated experiments (n=7 and n=5); means±SEM

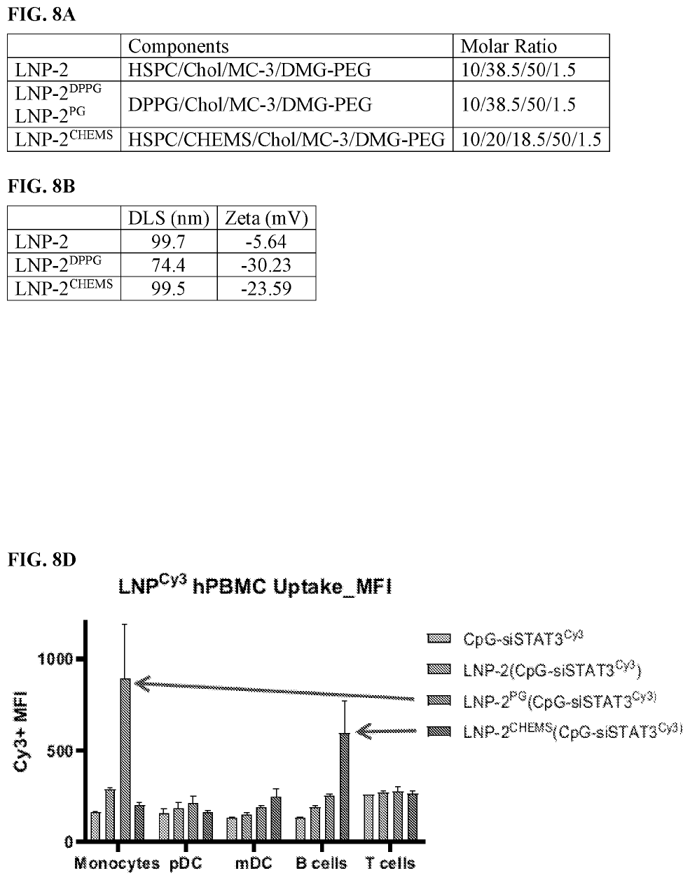

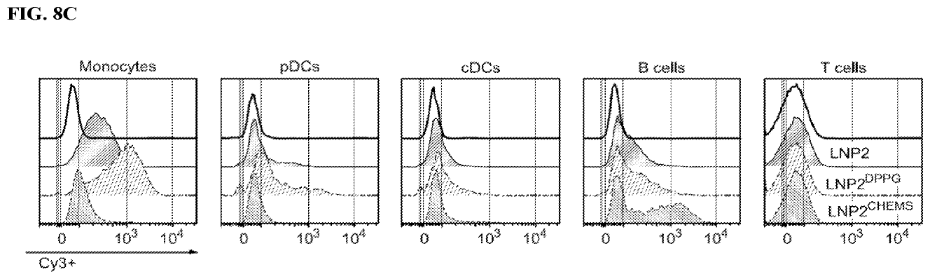

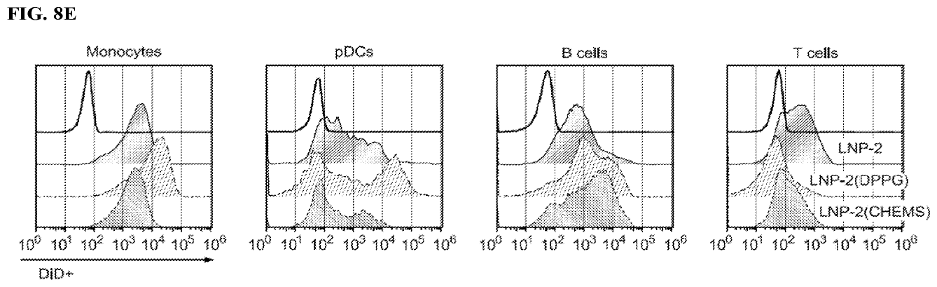

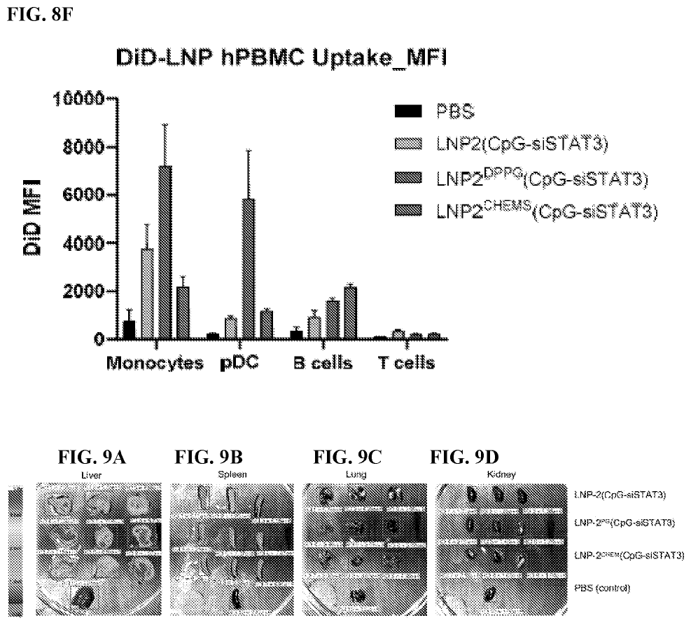

FIG. 8A is a table of molar ratios of individual lipid components for various LNP formulations, the sum of all components equal to 100. The LNP variants such as LNP2^{DPPG} or LNP2^{CHEMS} were derivatives of the original LNP2 formulation with the N to P ratio of 6 with 1.5% of DMG-PEG. FIG. 8B is a table of physiochemical characteristics of cell selective formulations. Each formulation was characterized on their size, morphology, dispersity, and surface charge. Shown are dynamic light scattering measurements (DLS) and zeta potential of each formulation. Phosphatidyl-glycerol (DPPG) and cholesteryl hemisuccinate (CHEMS) have anionic polar headgroup and should lead to negative surface charge if incorporated in the LNPs.

With reference to FIGS. 8C-8F: For ex-vivo uptake experiments, two variants of fluorescently labeled LNP formulations were prepared. Fluorescently labeled CpG-STAT3siRNA were loaded in LNPs with the indicated lipid formulation. Fluorescently labeled lipophilic dye (DiD) were incorporated into LNPs with indicated lipid formulation. After loading quantification, equal oligo molar concentration (100 nM) of LNPs (LNP2, LNP2^{DPPG}, LNP2^{CHEMS}) were added to 2×10⁶ freshly isolated human PBMC from healthy donors (n=3). Cells were collected after 4 h of incubation and stained for specific immune cell populations (monocytes: CD14+, pDC: CD303a+, mDC: CD1c+, B cells: CD19+, T cells: CD3+). Summary plots for the mean fluorescent intensity of LNP+ immune cell population were shown on the right; means±SD. Note: Similar uptake pattern were observed after labeling LNP oligonucleotide cargo or directly LNPs indicating successful delivery of intact cargo.

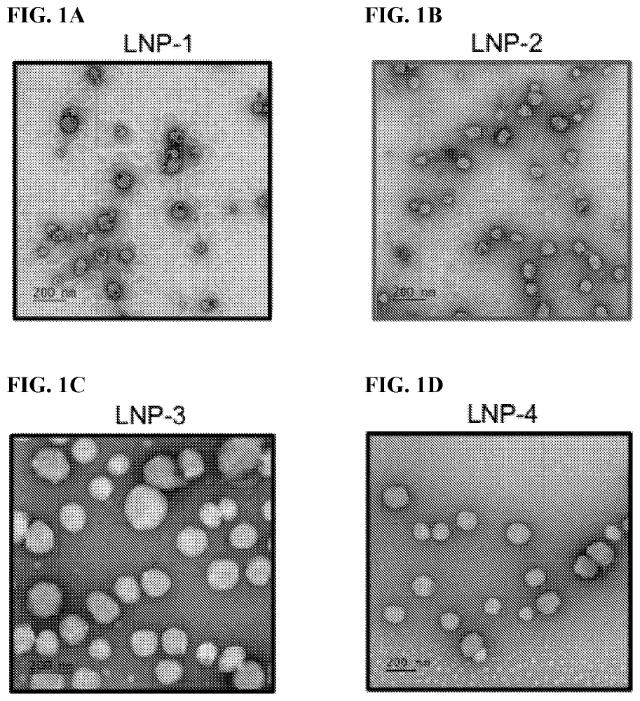

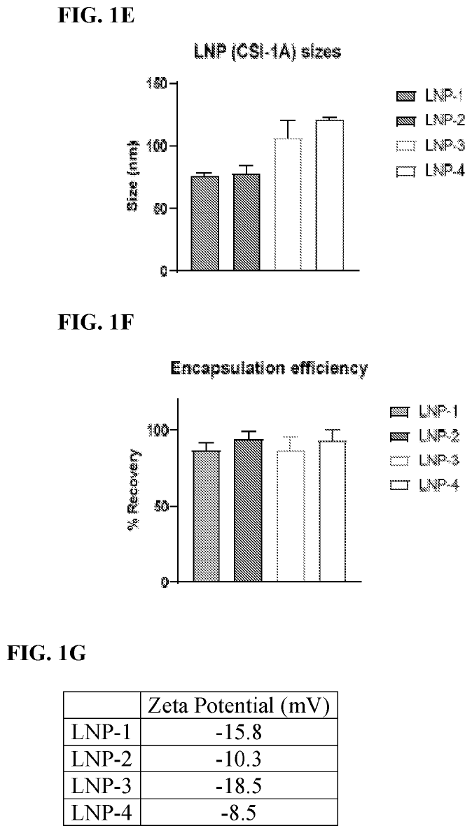

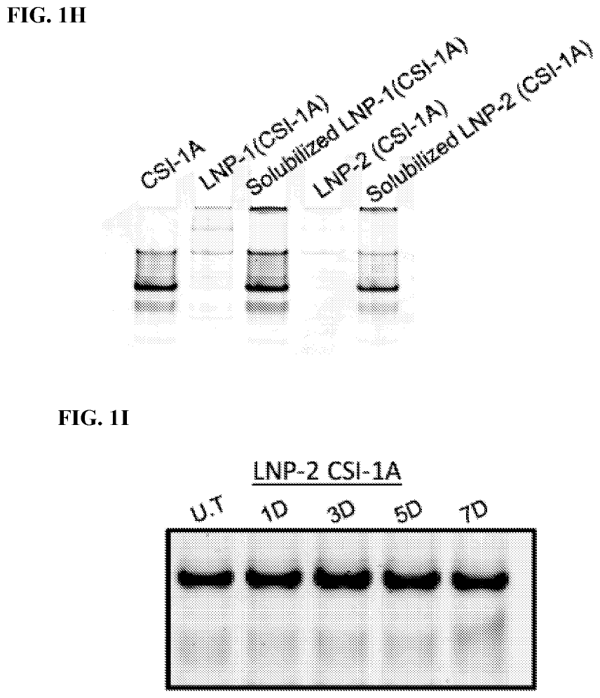

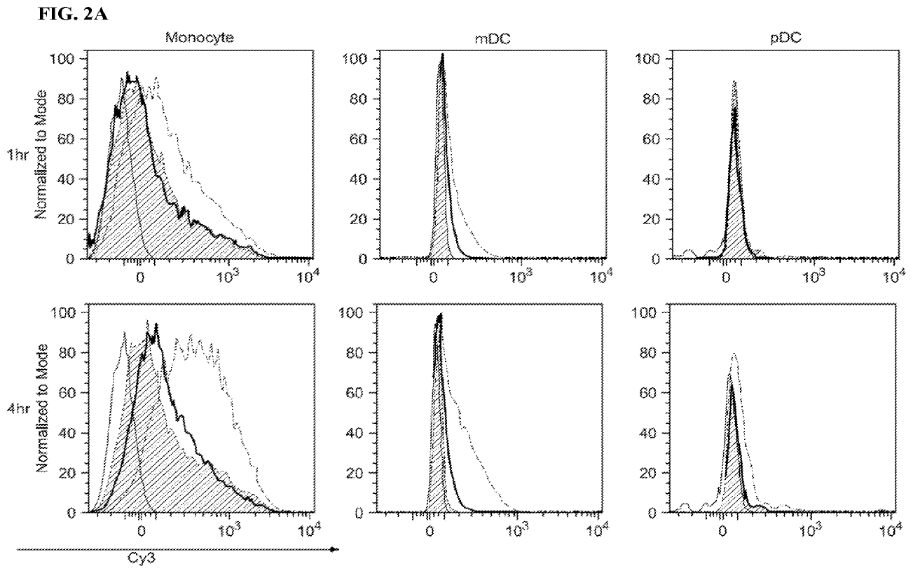

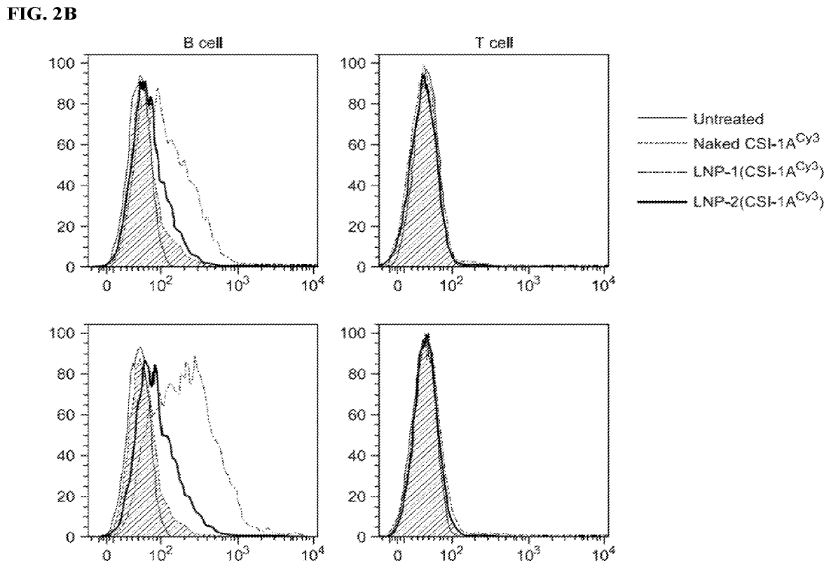

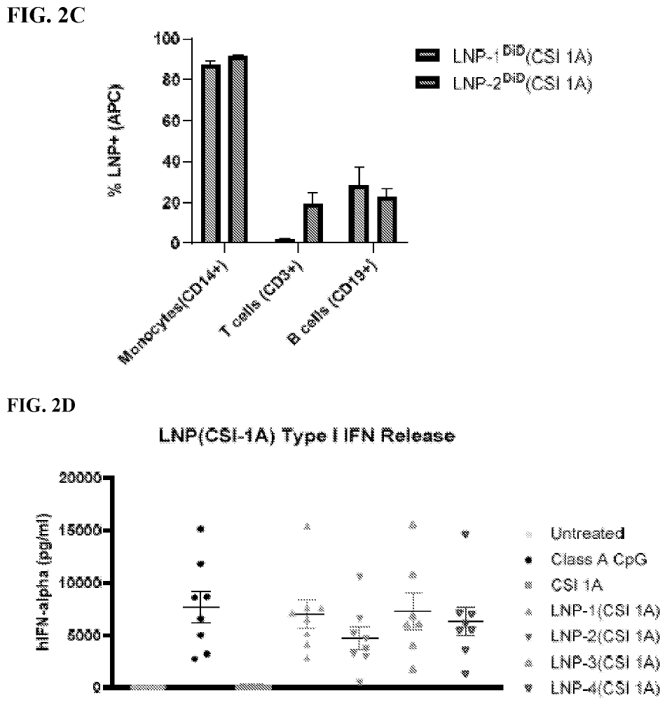

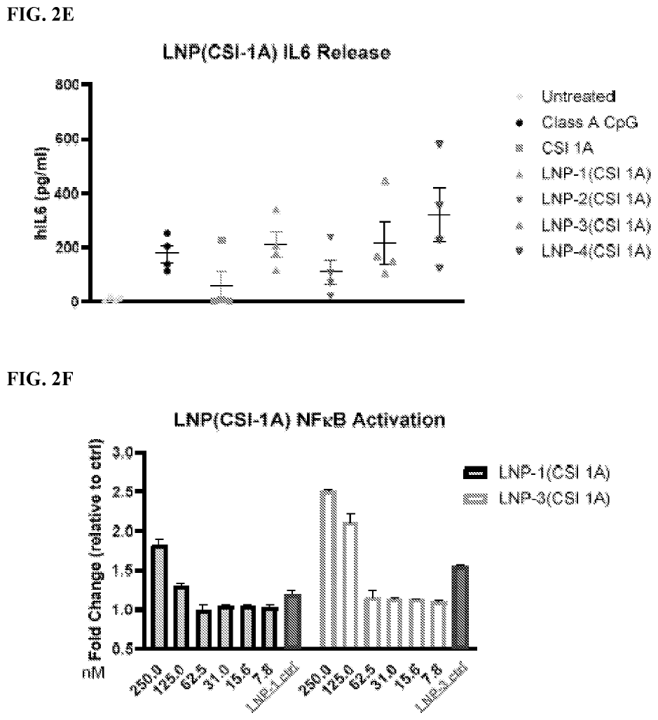

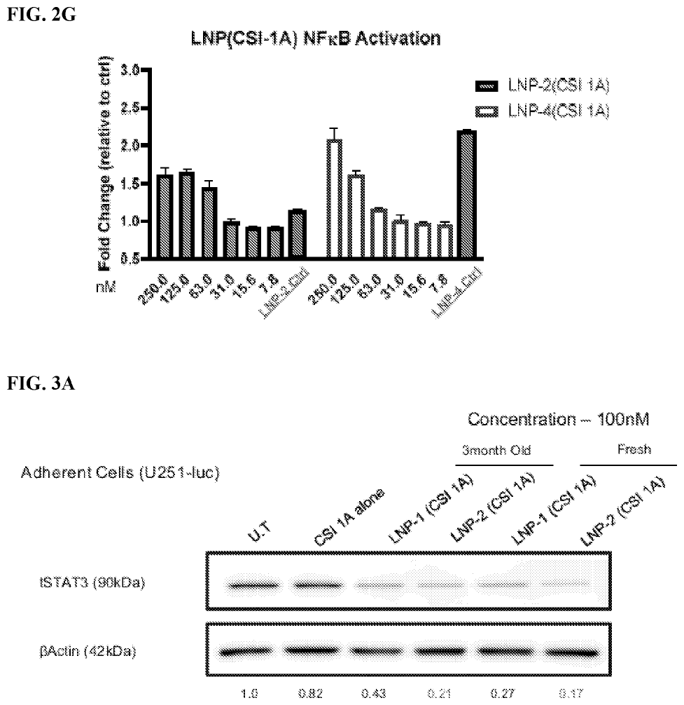

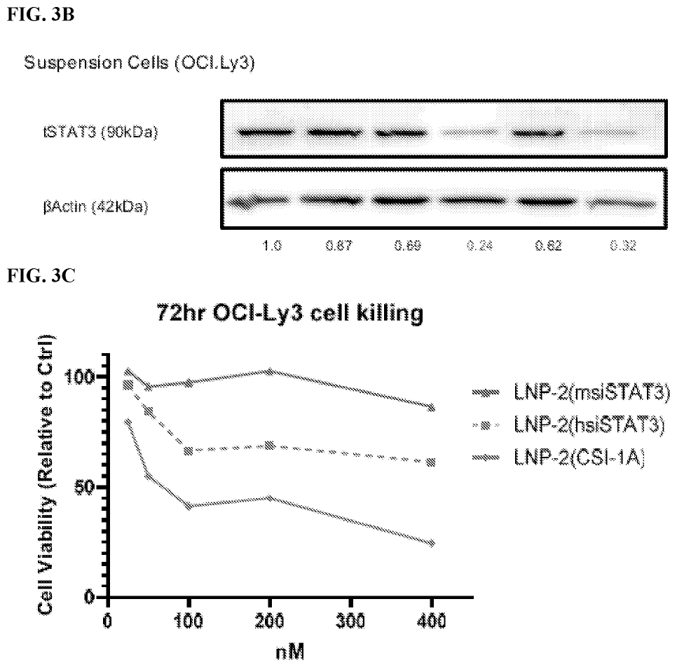

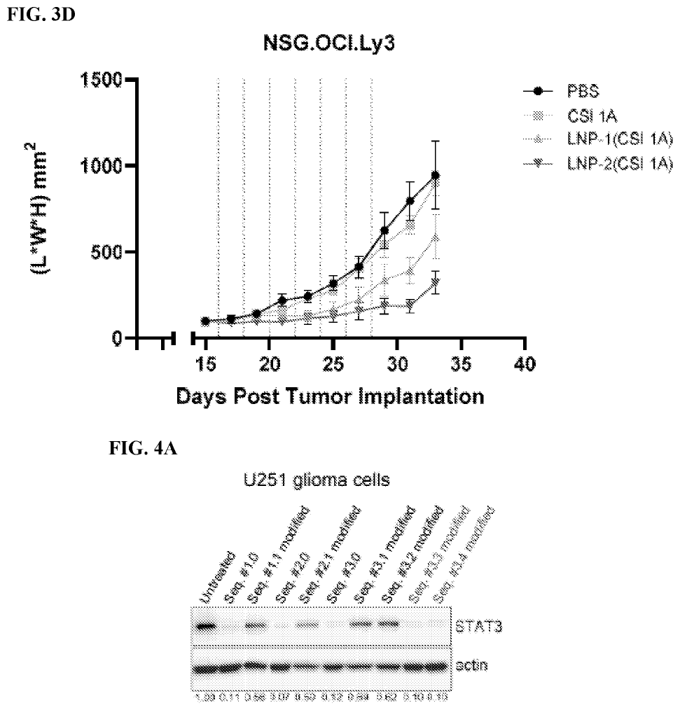

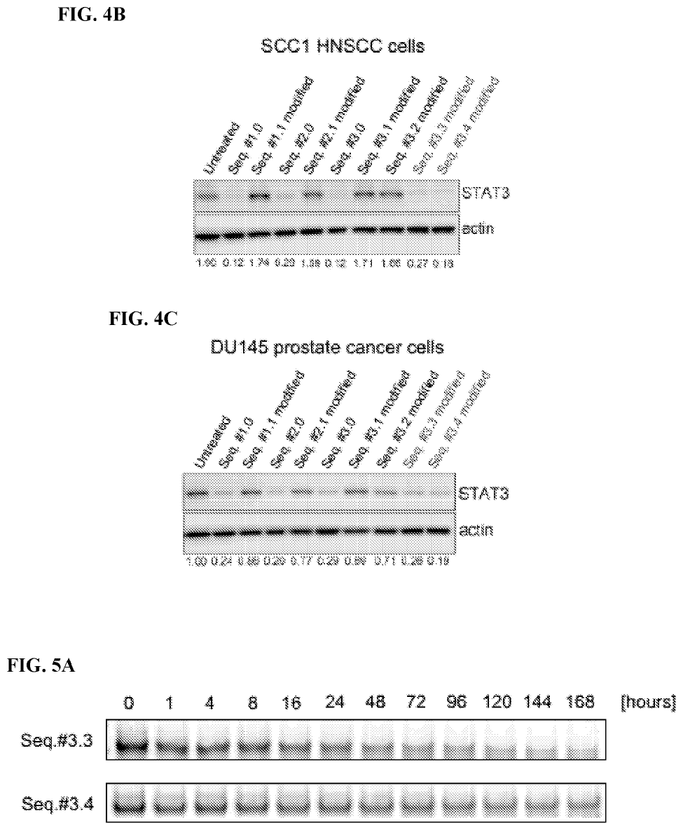

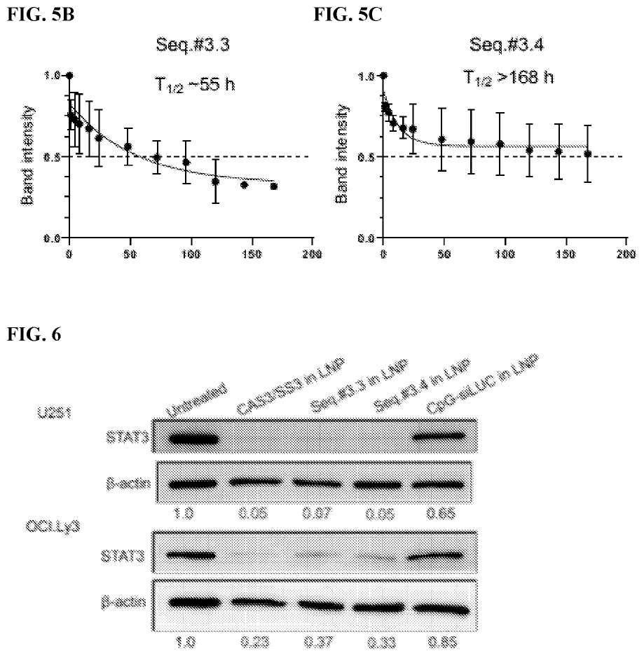

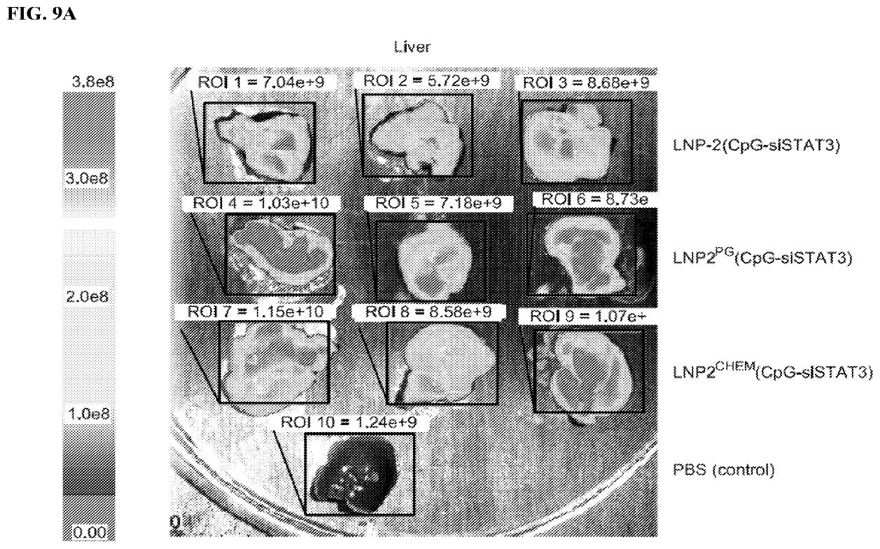

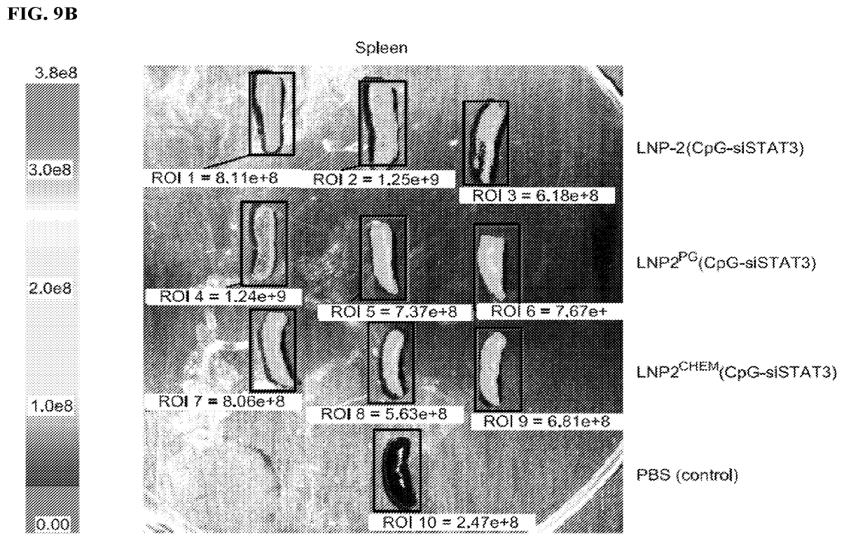

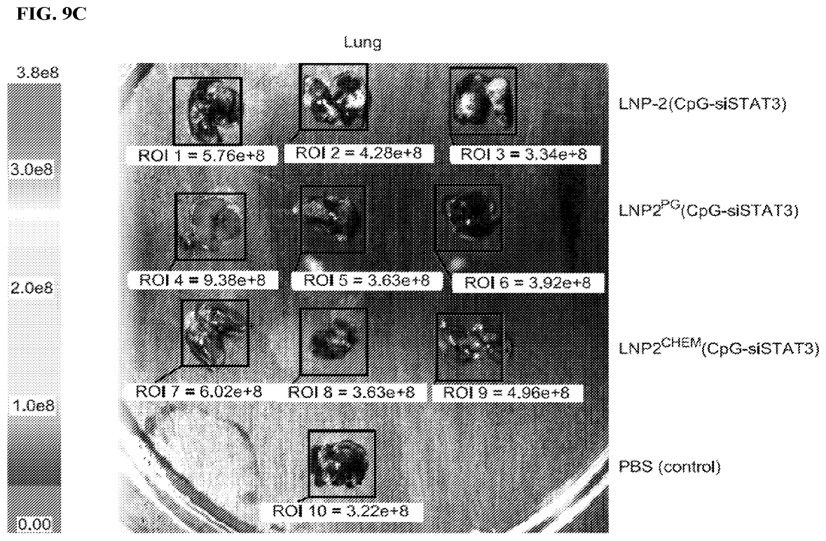

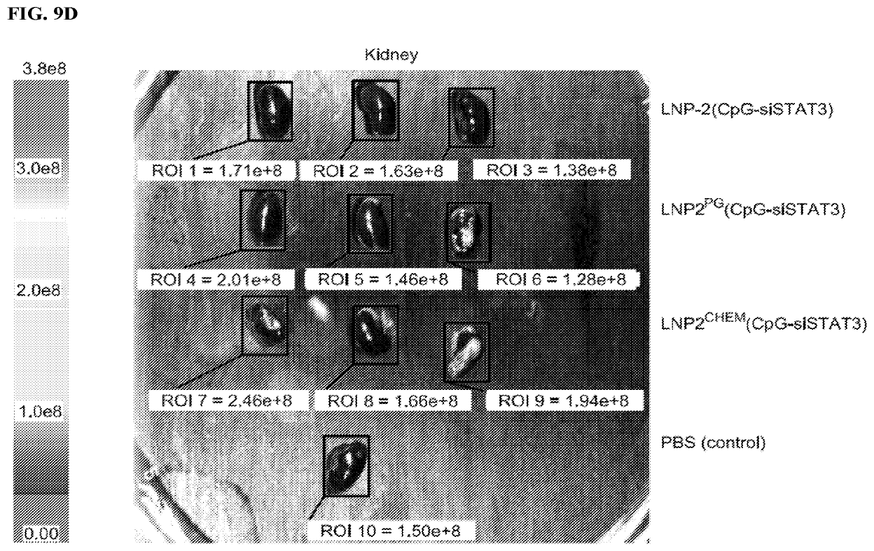

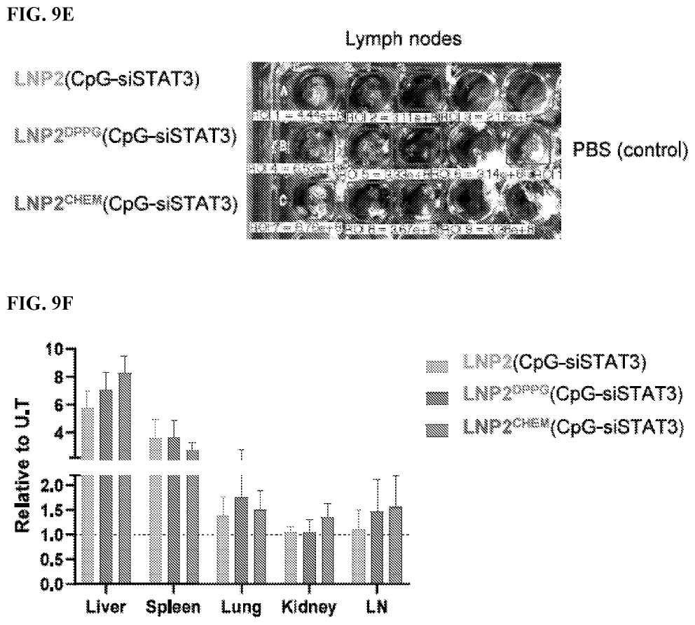

With reference to FIGS. 9A-9F, 1×10⁶ AML cells were implanted to C₅₇BL6 mice (8-12 weeks old) and various LNP formulations (refer to FIG. 2. A, and all LNP formulations were loaded with CpG-siSTAT3) labeled with DiD (0.15% mol/mol) lipophilic dye (1 mg/kg) were administered retro-orbitally/intravenously. After 3 h, mice were sacrificed, and major organs were collected for fluorescent imaging (excitation 605 nm/emission 690 nm with 5 s exposure at 20% power). Shown are fluorescently images adjusted equally for each organ collected (Liver; Spleen; Lung; Kidney; Peripheral lymph nodes). Each individual organ from various treatment groups were circled/analyzed for total fluorescent signal. FIG. 9F: the summarized fold increase of fluorescent signaling relative to untreated organs. Shown are means±SD (n=3). DiD labeled LNPs (CpG-siSTAT3) were found distribute similarly to organ level with exception of distribution to lymph nodes for LNP2^{DPPG}.

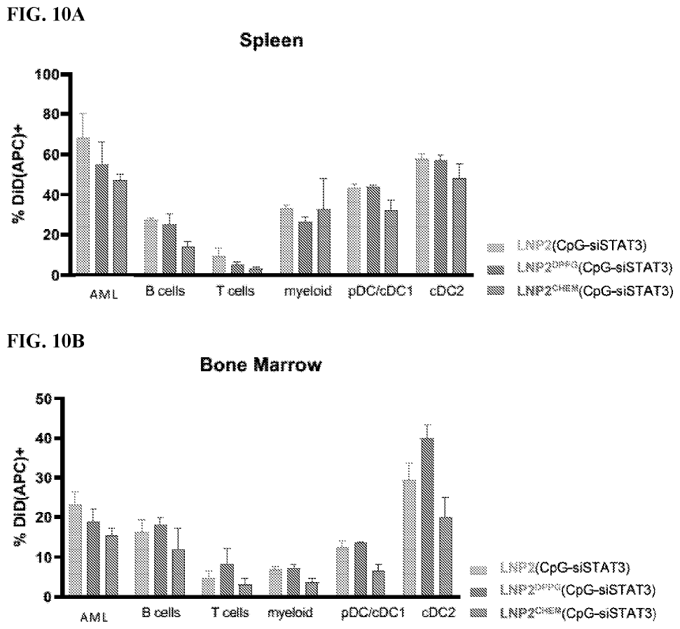

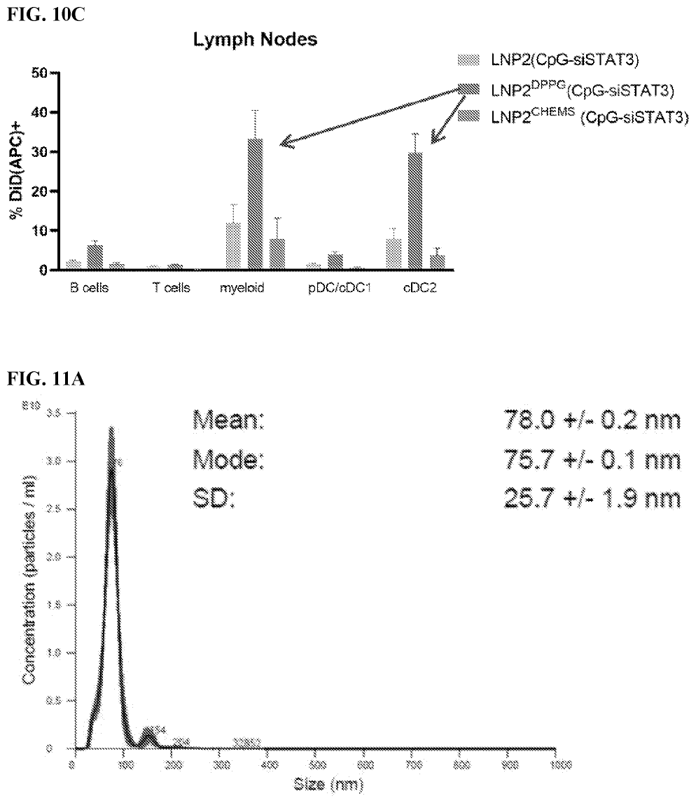

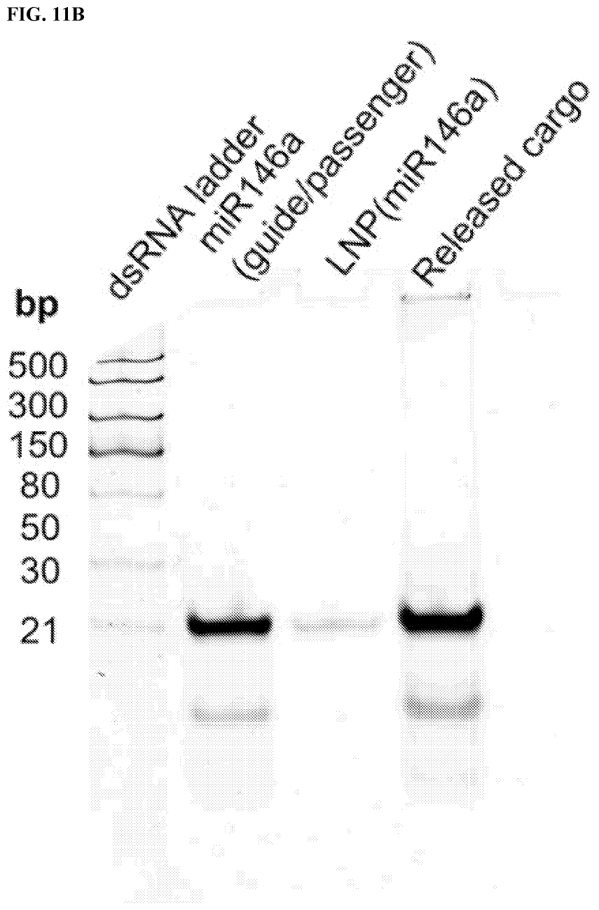

With reference to FIGS. 10A-10C, lymphoid organs were collected and processed to single cell suspensions for flow cytometry analysis. Briefly, total white blood cells were isolated and stained for immune cell populations (AML: eGFP+, B cells: CD19+, T cells: CD3+, monocytes/macrophages: CD11b+/CD11c−, pDC/cDC1: CD11b−/CD11c+, cDC2: CD11b+/CD11c+, shown are means±SD (n=3).

### Example 3

MicroRNA (miRNA) dysregulation is known to be associated with a variety of human diseases, including cancers and immune disorders. MiR146a represents one of the best characterized regulators of the immune response, as well as cellular survival through the negative feedback inhibition of nuclear factor-kappa B (NF-κB) signaling in myeloid cells. Restoration of miR146a levels would be an attractive therapeutic strategy for reducing exaggerated immune responses or to prevent certain types of blood cancers. However, delivery of synthetic miRNA mimics to target myeloid cells remains challenging. Here, we describe an optimized lipid nanoparticle (LNP) strategy for the delivery of miRNA mimics to myeloid immune cells and provide detailed protocols for characterization of LNP complexes and their biological activity. The encapsulation of miR146a within a lipid complex protects the nucleic acid from nuclease degradation, while allowing for rapid uptake by target myeloid immune cells. The strategy results in an efficient inhibition of target interleukin (IL) 1 receptor associated kinase 1 (IRAK1) and tumor necrosis factor receptor associated factor 6 (TRAF6) protein expression, thereby resulting in reduced NF-κB activity in mouse macrophages in vitro. The LNP-encapsulated miR146a effectively inhibits expression of IL-6, a major proinflammatory mediator downstream from NF-κB. This LNP-based strategy is suitable for testing of other miRNAs or RNA therapeutics targeting in myeloid immune cells.

MicroRNAs (miRNA) are endogenous, small non-coding RNAs that are responsible for regulating expression of multiple target genes. These miRNAs regulate gene expression through different modes of action, including sequence specific binding to 3′ untranslated regions of target gene mRNA to repress target protein expression, or by upregulating target gene expression through increased mRNA stability. Over 2,000 miRNAs have been discovered, and they are involved in the regulation of various biological pathways, including inflammation. Moreover, dysregulation of miRNAs has been associated with many immune disorders and with tumorigenesis. miR146a is one of the most well characterized miRNAs, and its dysregulation can lead to various severe consequences such as autoimmune disorders. However, the delivery of miR146a to its target cells posts a challenge. Su et al, Blood. 135(3):67-180 (2020) demonstrated that direct conjugation of CpG oligonucleotides with miRNAs can enhance the delivery to myeloid cell populations and resolve the acute inflammation response in mice. Here, we describe an attractive and enhanced strategy to deliver miR146a mimic to immune cells through encapsulation in lipid nanoparticles (LNPs).

LNPs have emerged as an attractive tool for gene therapy as underscored by recent rapid approvals of COVID-19 mRNA vaccines and by an earlier approval of ONPATTRO® (patisiran by Alnylam), which was the first LNP (siRNA) product approved in the market. LNPs are generally composed of four different components, which are helper lipids, polyethylene glycol, cholesterol and an ionizable/cationic lipid. Ionizable lipid forms complex with negatively charged oligonucleotides and alongside with other components to forms a stable, monodisperse lipid complex. These lipid complexes have been characterized and studied in pre-clinical and clinical settings. Here, we demonstrate that miR146a encapsulated LNPs can be readily taken up by myeloid cells, and induce a cargo specific response in macrophage cell lines as well as in splenocytes.

Encapsulation of miRNA-146a mimic (miR146a) into Lipid Nanoparticles with Quantification and Validation. Commercially available lipids (Avani, MedChemExpress): L-a-phosphatidylcholine, hydrogenated (Soy) (HSPC), cholesterol, D-Lin-MC3-DMA (MC3), and 1,2-dimyristoyl-rac-glycero-3-methoxypolyethylene glycol-2000 (DMG-PEG). NanoAssmblr benchtop unit (Precision Nanosystem). MiR146a sequence miR146a-mimic (guide) SEQ ID NO: 16; miR146a-mimic (passenger) SEQ ID NO: 17. DN/RNase free H₂O. Ethanol 200 proof. Amicon ultracentrifugation tubes with 10 kDa cutoff (EMD Millipore). Quantification of the encapsulated oligonucleotide using Quant-iT Ribogreen RNA Assay kit (ThermoFisher). Nanoparticle tracking analysis using NS300 (Malvern).

Polyacrylamide (PAGE) Gel Reagents: 10× Tris-Borate-EDTA buffer, BioReagent, 40% Acrylamide/Bis-acrylamide solution (19:1), Gel red dye (Biotum), tetramethylethylene-diamine, Double-stranded RNA molecular weight marker, RNA loading dye (2×), DNA loading dye (6×).

Western Blotting Reagents: Western blot assembly unit, Sample protein concentrations were determined using Pierce BCA protein analysis kit, PageRuler prestained protein ladder, Pierce PVDF transfer membrane, Immobilon blotting filter paper, Rabbit IRAK1-specific antibody (e.g, Cat. No. D51G7, Cell Signaling Technology), Rabbit TRAF6-specific antibody (e.g, Cat. No D21G3, Abcam), Goat anti-rabbit IgG H&L, Monoclonal 8-actin-specific and peroxidase-conjugated antibody, SuperSignal West Femto Maximum ECL detection reagent, Oligofectamine™ transfection reagent, Proteinase inhibitor (complete), Phosphatase inhibitors (PhosSTOP), Wash buffer: tris-buffered saline containing 1% Tween 20 (TBST), Blocking solution: 5% milk in TBST.

NF-KB Reporter Assay Using RAW-Blue™ Cells. Quanti-Blue solution for the colorimetric enzyme assay. Cytation 3 Micro-Plate Reader (BioTek). Clear bottom 96-well plate. GibCO™ Dulbecco's Modified Eagle's Medium (DMEM), high glucose. Fetal Bovine Serum (FBS). Antibiotic-Antimycotic (100×). GlutaMAX™ supplement.

LNP(miR146a^{Cy3}) Uptake by Primary Mouse Splenocytes. Mice: 8-12 weeks old C₅₇BL/6J mice (e.g., Jackson Laboratory). DNAse I grad II, from bovine pancreas. Collagenase D. 70 pm cell strainer. 1 mL syringe plunger. Red blood cell lysis buffer: NH₄Cl (150 mM), KHCO₃ (10 mM), Na₂EDTA dissolved in H₂O (pH 7.4). Single-cell suspension of fresh splenocytes in 10% FBS, 1% glutamine, 1% antibiotic-antimycotic supplemented DMEM at 37° C. Collection tube: round-bottom polystyrene tubes. Staining buffer: 1× phosphate buffered solution (PBS), 2% FBS, 0.1% NaN₃. CD16/CD32 monoclonal antibody. Fixable viability dye (aqua), mouse CD11b, CD11c, CD3, CD19. Flow Cytometer (e.g., Attune, Thermo Fisher).

The Assessment of IL-6 Splenocyte Cytokine Levels. MiR146KO mice-B6.Cg-Mir146tm1.1Bal/J (Jackson Laboratory). Lipopolysaccharide (LPS) from Salmonella enterica serotype. IL-6 mouse uncoated ELISA kit. STOP buffer: add 1 mL concentrated sulphuric acid (H₂SO₄) to 17.2 mL of double-distillated water. Cytation 3 Micro-Plate Reader (BioTek).

Formulation of miR146 into Lipid Nanoparticles. Reconstitute lyophilized pellet of miR146a with DN-/RNase-free water to make 1 mM stock solution, mix well at 37° C. for 5 min. Hybridize the miR146a (guide/passenger duplex) by mixing equal molar amount of each oligo and incubate at 80° C. using dry bath for 5 min, then let the solution cool down to room temperature. Quantify the oligo concentration using Cytation-3 multi-plate reader. Prepare each component of lipids by dissolving measured lipids (>5 mg) in ethanol and heat at 50° C. for 15 min, pipette up and down in between to allow better heat distribution and solubilization. Prepare the combined lipid formulation (Table 8) by mixing all lipid components in ethanol (prepare extra based on the oligonucleotide amount desired to encapsulate). Prepare the oligonucleotide solution by diluting it to 50 mM in citrate buffer at 0.133 mg/mL (250 μL lipid mixture and 750 μL oligonucleotide in citrate buffer for total of 1 mL of LNPs). Prepare the LNP formulation using Nanoassmblr bench top unit with total flow rate 9 mL/min, total flow rate ratio 3:1, aqueous phase at 1.5 mL and organic phase at 0.5 mL, initial waste is 0.25 mL and end waste is 0.05 mL. Upon completion of mixing of lipid with oligonucleotide components, add an excess amount of 1×PBS to the collection tube (top up to 15 mL). Transfer the LNP(miR146) product into a 10 kDa Amicon microtube and centrifuge at 2,000×g for 20-30 min at 16° C. Discard the flow through and add 14 mL PBS to the top unit, repeat the centrifuge process. Collect the product, LNP(miR146a) into a 1.5 mL Eppendorf tube and store at 4° C. The protocol focuses on the use of NanoAssmblr benchtop equipment for standardized LNP preparation. However, LNPs can also be prepared using more labor-intensive, manual methods if a microfluidics device isn't available as described by others.

TABLE 8
LNP composition (for 250 μL total volume)

| | MW | Concentration (mg/mL) | Mol % | Volume (μL) |
|---|---|---|---|---|
| HSPC | 783 | 10 | 10 | 28.4 |
| Cholesterol | 386.66 | 10 | 38.5 | 53.6 |
| MC3 | 642 | 10 | 50 | 115.5 |
| PEG-DMG | 2805 | 10 | 1.5 | 15.1 |
| Ethanol | | | | 37.25 |

Characterization of LNP Formulation. Extract the miR146a from LNPs using 10 μL of 2% Triton-X for each 10 μL of LNP(miR146a) and incubate at 37° C. for 15 min. The released oligonucleotides should be diluted 10× using DN-/RNase-free water and then diluted 200× in TE buffer. Prepare standards with naked miR146a by first diluting the oligo stock to 200 μg/mL, then add 10 μL of 2% Triton-X together with 10 μL miR146a. Dilute the mixture to 50-100 ng/mL with 1× TE buffer. Pipette 100 μL standards (serial diluted) and testing sample into a black well/clear bottom plate and add 100 μL Quant-it solution (1:2,000 in TE buffer) to all wells. Incubate in the dark for up to 5 min then read using excitation/emission 480/520 nm. Calculate concentration of the LNP-encapsulated oligonucleotide based on a standard curve. Prepare sample by diluting stock LNP (approx. 2.5 mg/mL of lipid) 400× with PBS. Analyze the LNP size and particle concentration were obtained by performing three 60 sec runs at a continuous flow rate of syringe pump speed (30) NTA analysis (NS300 Malvern) (FIG. 11A).

Validation of miR146 Encapsulation in LNPs Using Gel Electrophoresis. Assemble the gel plates in the gel casting chamber, prepare 15% PAGE gel (see Table 9) and pour quickly between gel plates using pipette, insert comb and leave to polymerize for about 30 min. Place the gel in the electrophoresis apparatus according to manufacturer's instruction and fill the chamber with 1×TBE. Extract the miR146a from LNPs using 10 μL of 2% Triton-X for each 10 μL of LNP(miR146a) and incubate at 37° C. for 15 min. The released oligonucleotides should be diluted 10× using DN-/RNase-free water and then diluted 200× in TE buffer. Then add 20 μL of 2×RNA loading buffer (released cargo). Dilute miR146a to 200 μg/mL and mix with an equal amount of 2×RNA loading buffer. Mix 10 μL of LNP(miR146a) with 10 μL DNA loading dye (FIG. 11B). Load 2 μL of each testing sample together with double-stranded RNA molecular marker and run the gel at 110 V for 1 hour. Remove the gel and transfer into a dish containing 1×TBE with Gel red staining solution (diluted 1:1,000 in TBE buffer), incubate for 10-15 min, then examine the gel using ChemiDoc MP imaging system (FIG. 11B). Freshly-prepared LNPs can be filtered through a 0.2 μm filter to sterilize the stock solution before use or storage. Generally, LNP formulations generated using the described method can be stored at 4° C. for up to 3 months without significant loss of miRNA functionality. Small size LNPs (<200 nm) are usually transparent in solution with a slight blueish tint. If the solution turns opaque or aggregates are visible, it's an indication of destabilized particles.

TABLE 9
15% PAGE gel

| | Volume | |
|---|---|---|
| Acrylamide/Bis-acrylamide 40% | 5.6 | mL |
| H₂O | 7.7 | mL |
| 10 × TBE | 1.5 | mL |
| 10% APS | 200 | μL |
| TEMED | 10 | μL |

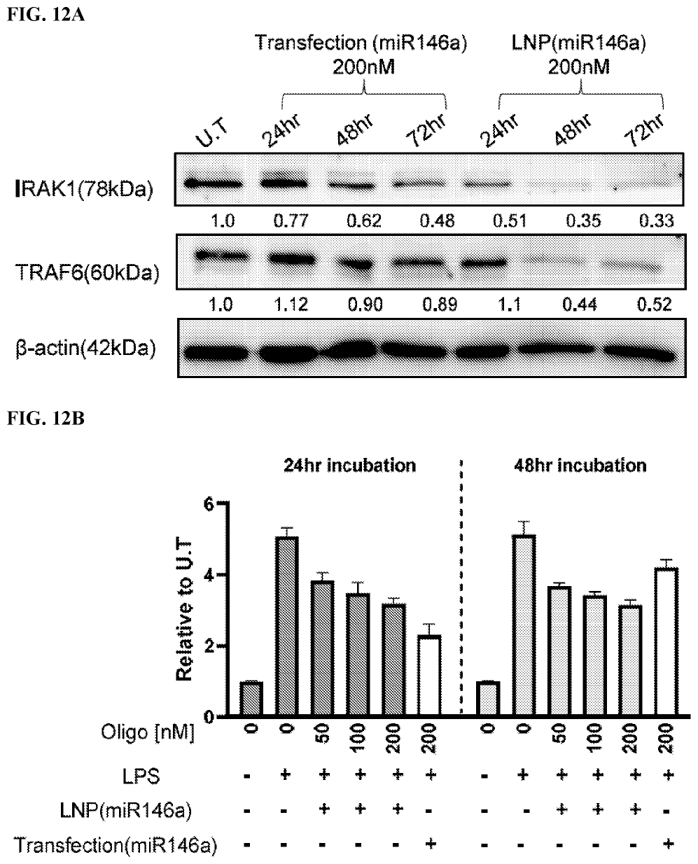

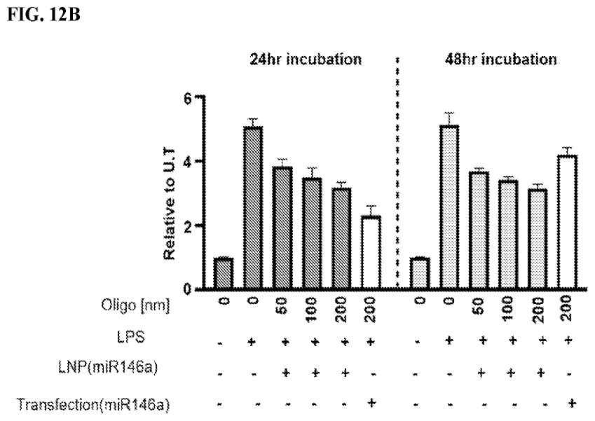

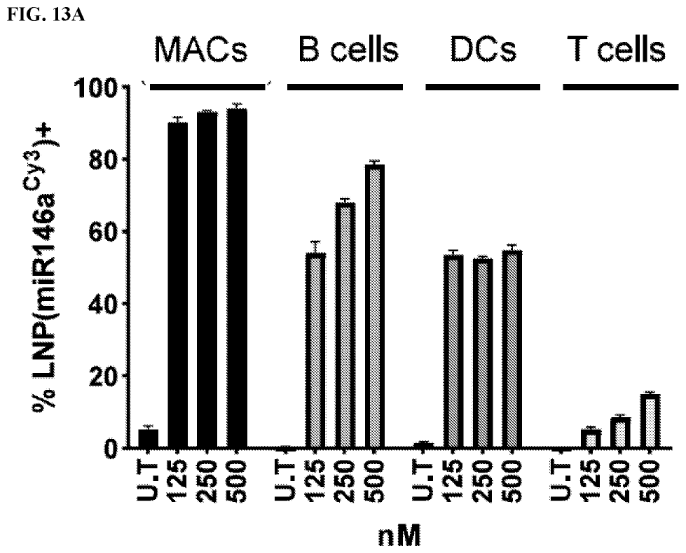

Functional Verification of LNP(miR146) in Mouse Macrophages by Western Blot. Plate and culture RAW264.7 macrophages on a 6-well plate overnight at a desired density (10⁵ cells/well) in DMEM supplemented with 10% FBS, 1% glutamine, 1% antibiotics-antimycotics. Next day, add pre-complexed oligofectamine/miR146a to cultured wells for 6 hours and replace with fresh medium. Add a desired concentration of LNP(miR146a) (usually 50-200 nM). On the day of collection, carefully collect the cells after incubation on ice for 10 min to allow cell detachment, and pipette the medium to facilitate cell detachment. Spin the cells down at 500×g for 3 min, wash the cell pellet with 1×PBS and repeat centrifugation. Resuspend cell pellet in RIPA lysis buffer supplemented with proteinase (complete) and phosphatase inhibitors (PhosSTOP) (Table 10) at a desired volume (e.g., 100 μL), and allow for complete lysis of the cells by incubating on ice for 30 min. Remove the DNA pellet by centrifugation at maximal speed for 25 min at 4° C. and remove the pellet using a pipette. Read protein concentration by loading 5 μL of standards (e.g., Pierce BCA protein analysis kit) or samples in duplicate to a 96-well plate and add 195 μL of reading solution. Incubate at 37° C. for 20 min and read absorbance at 560 nm. Assemble the gel plates in the gel casting chamber, and prepare SDS-PAGE gels (Table 11). Fill the gel chamber with Tris running buffer, load 5 μg of protein sample (denatured with 20% (3-mercaptoethanol in 4× loading dye) to each well. Run the gel at 90 V for 30 min, then 110 V for 90 min. Prepare transfer membrane by activate in methanol for 30 sec then soak in transfer buffer. Prepare transfer cassette and carefully layer gel, a blotting membrane, and a blotting paper. Run the transfer with 110 V for 1 hour at 4° C. Add blocking solution to the blotting membrane for 30 min. Cut the gel based on protein ladder position, and incubate with primary antibodies (anti-IRAK1 1:3000; anti-β-actin 1:100,000 in TBST with 2.5% milk) overnight at 4° C. Add wash buffer to the blotting membrane with rinsing over 5 times, then incubate with secondary antibodies for 40 min. Repeat washing steps and image the membranes. Analyze the target bands relative to control ((3-actin) using ImageLab software (see FIG. 12A).

TABLE 10
RIPA Buffer

| | Stock Solution | For 100 mL | |
|---|---|---|---|
| 20 mM Tris pH 8.2 | 2M | 1 | mL |
| 158 mM NaCl | 5M | 3.16 | mL |
| 1% Triton X-100 | 100% | 1 | mL |
| 1% sodium deoxycholate | | 1 | g |
| 0.1% SDS | 20% | 0.5 | mL |
| 5 mM EDTA | 0.5M | 1 | mL |

TABLE 11
8% SDS-PAGE gel for western blots

| | For Two Gels (μL) |
|---|---|
| Acrylamide/Bis-acrylamide 40% | 2,000 |
| Tris pH 8.8 | 2,500 |
| SDS 20% | 50 |
| H₂O | 5,400 |
| APS (10%) | 100 |
| TEMED | 5 |
| Total Volume | 10,000 |

TABLE 12
Concentrating Gel (5%); remove isopropanol
and wash with H2O twice

| | For Two Gels (μL) |
|---|---|
| Acrylamide/Bis-acrylamide 40% | 750 |
| Tris pH 6.8 | 760 |
| SDS 20% | 30 |
| H₂O | 4,400 |
| APS (10%) | 60 |
| TEMED | 5 |
| Total Volume | 6,000 |

In Vitro RAW-Blue™ Assay to Verify On-Target Activity of LNP(miR146a). Plate 1-2×10⁴ RAW-Blue™ cells in 96 well plates with appropriate medium. On the next day, carefully remove the supernatant and replace with treatment medium. After desired incubation time (24-48 hours), remove the supernatant and replace with LPS containing medium (100 ng/mL) at 200 μL per well. After desired stimulation time (4-8 hours), remove 20 μL of supernatant to a new 96-well plate, add 180 μL of QuantiBlue solution. Use untreated cell supernatant as negative control, and LPS treated only cell supernatant as positive control. Incubate for 1-3 hours until visible color differences (purple to deep blue color transition). Read the plate at 625 nm and analyze as fold activation compared to untreated control (see FIG. 12B). RAW-Blue™ cells secrete enzyme SEAP that accumulates in the cell culture supernatant to be analyzed using a colorimetric reaction. It is important to optimize conditions of the experiment for the specific type of stimulation and miRNA type. For example, LPS rapidly activates RAW-Blue™ cells but it takes 4-8 h to accumulate detectable amounts of SEAP in the medium.

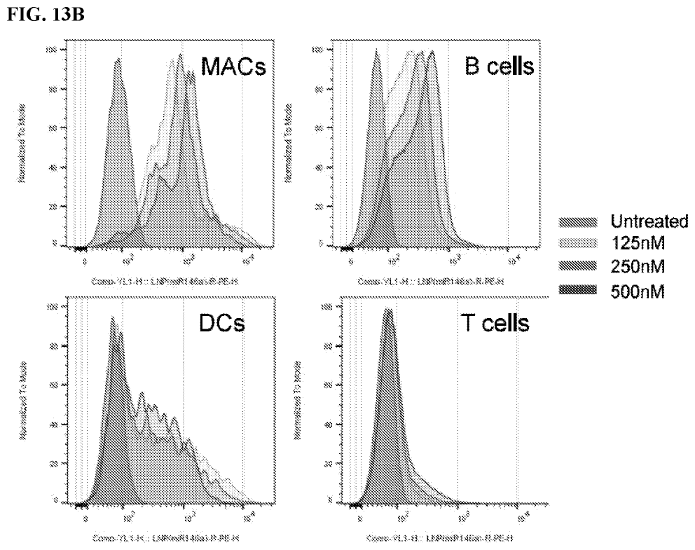

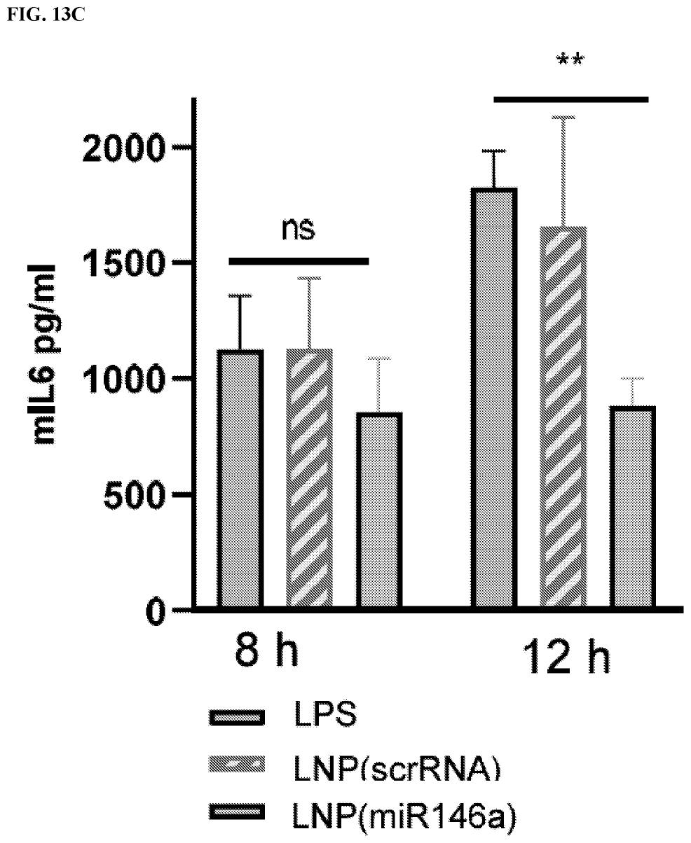

Cell-Selective Uptake of LNP(miR146a) by Primary Mouse Immune Cells. Harvest spleens from C57BL/6 mice, and transfer to ice-cold culture medium. Prepare 70 μm cell strainer and place on top of a 50 mL conical tube. Transfer harvested spleen into Petri dishes and wash twice with Hank's Balanced Salt Solution. Add 5-10 mL of Collagenase D (400 U/mL) and DNase I (1 mg/mL) and cut spleen into small cubes (−1-2 mm in diameter) using surgical scissors, and incubate for 15-30 min at 37° C. Neutralize the reaction by adding excess medium, carefully push through the digested tissue through cell strainer with a syringe plunger. Centrifuge the splenocytes at 400×g for 5 min at 4° C. Resuspend the pellet in 3-5 mL of red blood cell lysis buffer and incubate at room temperature for 5 min. Quench the reaction by adding the same volume of medium. Remove red blood cell lysis buffer from splenocytes by centrifugation at 400×g for 5 min at 4° C. twice. Resuspend the pellet with 5 mL of DMEM culture medium supplemented with 10% FBS, 1% L-glutamine. Count splenocytes and plate 2-3×10⁶ cells per well (48-well plate) in 800 μL medium. Add labeled oligonucleotide formulations at desired concentrations LNP(miR146a^{Cy3}) (200-500 nM). Incubate 4-8 h. Collect cells, resuspend each sample in 100 μL of PBS containing 1 pL fixable aqua (or other viability dye). Stain cells for 15-30 min on ice, wash once with 2 mL PBS and resuspend in the staining buffer containing Fey Block (CD16/32) and specific antibody cocktail. Stain cells for 30 min on ice, wash twice with 2 mL staining buffer. Analyze cell uptake using flow cytometry (as in FIGS. 13A-13B).

Cytokine Assay to Verify Biological Activity of LNP(miR146a). Harvest spleens from miR146a KO mice, and transfer to ice-cold culture medium. Acquire single cell suspension splenocytes. Count the splenocytes and plate 2-3×10⁶ cells per well (48-well plate) in 800 pL medium. Add oligonucleotide formulations at desired concentrations LNP(miRNA) 50-200 nM. Incubate overnight. Stimulate the splenocytes by directly addition of LPS (100 ng/ml). Collect culture supernatant (50 μL) at 8, 12 or 24 h after stimulation and immediately store at −80° C. until ready to run ELISA (FIG. 13C). Refer to manufacture instruction to perform ELISA, read absorbance at 450 nm (IL-6 levels) and 570 nm (background subtraction). An excess of ionizable lipids can decrease viability of target cells. If this becomes an issue, the percentage of MC3 lipids can be reduced or replaced by alternative ionizable lipids. It is also important to provide respective negative controls such as LNP formulation of a scrambled RNA or an unrelated miRNA oligonucleotide. The nuclease resistance of miRNA and siRNA oligonucleotides can be greatly enhanced by sequence modification using chemical modification of riboses in RNA nucleotides (e.g. 2′-O-methyl and 2′-fluoro modified nucleotides) in various patterns as described by others.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1-33. (canceled)

34. A lipid nanoparticle comprising:
(i) about 40 mole % to about 60 mole % of a cationic lipid:
(ii) about 5 mole % to about 15 mole % of a phospholipid;
(iii) about 28 mole % to 50 mole % of a sterol; and
(iv) about 0.1 mole % to about 4 mole % of a polyethylene glycol-lipid conjugate.

35. The lipid nanoparticle of claim 34 comprising:
(i) about 48 mole % to about 52 mole % of the cationic lipid;
(ii) about 8 mole % to about 12 mole % of the phospholipid;
(iii) about 35 mole % to 42 mole % of the sterol; and
(iv) about 0.1 mole % to about 2 mole % of the polyethylene glycol-lipid conjugate.

36. The lipid nanoparticle of claim 35 comprising:
(i) about 50 mole % of the cationic lipid;
(ii) about 10 mole % of the phospholipid;
(iii) about 38.5 mole % of the sterol; and
(iv) about 1.5 mole % of the polyethylene glycol-lipid conjugate.

37. The lipid nanoparticle of claim 35 comprising:
(i) about 50 mole % of the cationic lipid;
(ii) about 10 mole % of the phospholipid;
(iii) about 39.5 mole % of the sterol; and
(iv) about 0.5 mole % of the polyethylene glycol-lipid conjugate.

38. The lipid nanoparticle of claim 34, wherein the cationic lipid is a dilinoleic cationic lipid; wherein the dilinoleic cationic lipid is MC3, an MC3 derivative, DLinDMA, DLin-K-C2-DMA, DLin-K-C3-DMA, DLin-K-C4-DMA, DLin-K6-DMA, DLin-K-MPZ, DLin-K-DMA, DLin-C-DAP, DLin-DAC, DLin-MA, DLinDAP, DLin-S-DMA, DLin-2-DMAP, DLin-TMA, DLin-TAP, DLin-MPZ, DLinAP, DLin-EG-DMA, DLincarbDAP, or a mixture of two or more thereof.

39. The lipid nanoparticle of claim 34, wherein the phospholipid is DSPC, DPPC, DOPE, POPC, POPE, POPG, DPPE, DMPE, DSPE, MMPE, DMPE, DEPE, SOPE, EPC, HSPC, DPPG, or a mixture of two or more thereof.

40. The lipid nanoparticle of claim 34, wherein the sterol is cholesterol, cholestanol, cholestanone, cholestenone, coprostanol, cholesteryl-2′-hydroxyethyl ether, cholesteryl-4′-hydroxybutyl ether, cholesteryl hemisuccinate, or a mixture of two or more thereof.

41. The lipid nanoparticle of claim 34, wherein the polyethylene glycol-lipid conjugate is a polyethylene glycol having a molecular weight from about 1,000 Daltons to about 6,000 Daltons conjugated to a C₁₂-C₂₂ fatty acid lipid.

42. The lipid nanoparticle of claim 41, wherein the polyethylene glycol-lipid conjugate is DMG-PEG, DPPE-PEG, DPG-PEG, DSG-PEG, DSPE-PEG, or a mixture of two or more thereof.

43. The lipid nanoparticle of claim 34, wherein
(i) the cationic lipid is MC3;
(iii) the phospholipid is HSPC or DPPG;
(iv) the sterol is cholesterol or a mixture of cholesterol and cholesteryl hemisuccinate; and
(v) the polyethylene glycol-conjugated lipid is DMG-PEG, wherein the polyethylene glycol has a molecular weight of about 2,000 Daltons.

44. The lipid nanoparticle of claim 43, wherein the sterol is the mixture of cholesterol and cholesteryl hemisuccinate, and wherein the molar ratio of cholesterol to cholesteryl hemisuccinate is 2:1 to 1:2.

45. The lipid nanoparticle of claim 44, wherein the sterol is 18.5 mole % cholesterol and 20 mol % cholesteryl hemisuccinate.

46. The lipid nanoparticle of claim 34, wherein a plurality of the lipid nanoparticles have an average size from about 50 nm to about 150 nm.

47. The lipid nanoparticle of claim 34, further comprising a nucleic acid encapsulated within the lipid nanoparticle.

48. The lipid nanoparticle of claim 47, wherein the nucleic acid comprises SEQ ID NO: 7; SEQ ID NO:7 hybridized to SEQ ID NO:8; SEQ ID NO:8; SEQ ID NO:9; SEQ ID NO: 10; SEQ ID NO:11; SEQ ID NO:12; SEQ ID NO:13; SEQ ID NO:7 hybridized to SEQ ID NO: 8; SEQ ID NO:9 hybridized to SEQ ID NO:10; SEQ ID NO:11 hybridized to SEQ ID NO: 12; SEQ ID NO: 14; SEQ ID NO: 14 hybridized to SEQ ID NO:8; SEQ ID NO: 15; SEQ ID NO: 15 hybridized to SEQ ID NO:10; SEQ ID NO:6; SEQ ID NO:6 hybridized to SEQ ID NO: 12; SEQ ID NO:16; SEQ ID NO:17; or SEQ ID NO:16 hybridized to SEQ ID NO:17.

49. The lipid nanoparticle of claim 47, wherein the N/P ratio is from about 2:1 to about 8:1.

50. A pharmaceutical composition comprising the lipid nanoparticle of claim 34 and a pharmaceutically acceptable excipient.

51. A method for treating cancer in a patient in need thereof, the method comprising administering to the patient the lipid nanoparticle of claim 34.

52. A method of delivering a lipid nanoparticle to a myeloid cell, a lymphoid organ, or a tumor in a patient in need thereof, the method comprising administering to the patient the lipid nanoparticle of claim 34.

53. A nucleic acid comprising: (i) a CpG oligodeoxynucleotide bound to SEQ ID NO: 9; (ii) SEQ ID NO:9 hybridized to SEQ ID NO:10; (iii) a CpG oligodeoxynucleotide bound to SEQ ID NO:11; (iv) SEQ ID NO:11 hybridized to SEQ ID NO:12; (v) SEQ ID NO:15; (vi) SEQ ID NO:15 hybridized to SEQ ID NO:10; (vii) SEQ ID NO:6; (viii) SEQ ID NO:6 hybridized to SEQ ID NO:12; (ix) SEQ ID NO:9; (x) SEQ ID NO:10; (xi) SEQ ID NO:11; (xii) SEQ ID NO:12: (xiii) SEQ ID NO:7; (xiv) SEQ ID NO:7 hybridized to SEQ ID NO:8; (xv) SEQ ID NO: 14; or (xvi) SEQ ID NO:14 hybridized to SEQ ID NO:8.


SEQUENCE LISTING (17 sequences)

SEQ ID NO: 1 - RNA, 27 nt, Synthetic construct
  misc_feature 1..27: note = synthetic Polynucleotide
  source 1..27: mol_type = other RNA
  modified_base 1: mod_base = OTHER; note = uracil
  modified_base 5: mod_base = OTHER; note = uracil
  modified_base 8: mod_base = OTHER; note = uracil
  modified_base 10: mod_base = OTHER; note = uracil
  modified_base 12: mod_base = OTHER; note = uracil
  modified_base 13: mod_base = OTHER; note = uracil
  modified_base 14: mod_base = OTHER; note = uracil
  modified_base 16: mod_base = OTHER; note = uracil
  modified_base 22: mod_base = OTHER; note = uracil
  modified_base 23: mod_base = OTHER; note = uracil
  modified_base 27: mod_base = OTHER; note = uracil
tcagtcgtatctttctgcagcttccgt

SEQ ID NO: 2 - RNA, 25 nt, Synthetic construct
  misc_feature 1..25: note = synthetic Polynucleotide
  source 1..25: mol_type = other RNA
  modified_base 7: mod_base = OTHER; note = uracil
  modified_base 17: mod_base = OTHER; note = uracil
  modified_base 23: mod_base = OTHER; note = uracil
ggaagctgcagaaagatacgactga

SEQ ID NO: 3 - RNA, 21 nt, Synthetic construct
  misc_feature 1..21: note = synthetic Polynucleotide
  source 1..21: mol_type = other RNA
  modified_base 5: mod_base = OTHER; note = uracil
  modified_base 9: mod_base = OTHER; note = uracil
  modified_base 10: mod_base = OTHER; note = uracil
  modified_base 11: mod_base = OTHER; note = uracil
  modified_base 14: mod_base = OTHER; note = uracil
  modified_base 15: mod_base = OTHER; note = uracil
  modified_base 16: mod_base = OTHER; note = uracil
  modified_base 21: mod_base = OTHER; note = uracil
aggatgactttgatttcaact

SEQ ID NO: 4 - RNA, 23 nt, Synthetic construct
  misc_feature 1..23: note = synthetic Polynucleotide
  source 1..23: mol_type = other RNA
  modified_base 3: mod_base = OTHER; note = uracil
  modified_base 4: mod_base = OTHER; note = uracil
  modified_base 9: mod_base = OTHER; note = uracil
  modified_base 15: mod_base = OTHER; note = uracil
  modified_base 18: mod_base = OTHER; note = uracil
  modified_base 21: mod_base = OTHER; note = uracil
  modified_base 22: mod_base = OTHER; note = uracil
  modified_base 23: mod_base = OTHER; note = uracil
agttgaaatcaaagtcatccttt

SEQ ID NO: 5 - DNA, 20 bp, Synthetic construct
  misc_feature 1..20: note = synthetic Polynucleotide
  source 1..20: mol_type = other DNA
ggtgcatcgatgcagggggg

SEQ ID NO: 6 - DNA, 69 bp, Synthetic construct
  source 1..69: mol_type = other DNA
  modified_base 2: mod_base = OTHER; note = n appears after the nucleotide having the phosphorothioate modification
  modified_base 4: mod_base = OTHER; note = n appears after the nucleotide having the phosphorothioate modification
  modified_base 19: mod_base = OTHER; note = n appears after the nucleotide having the phosphorothioate modification
  modified_base 21: mod_base = OTHER; note = n appears after the nucleotide having the phosphorothioate modification
  modified_base 23: mod_base = OTHER; note = n appears after the nucleotide having the phosphorothioate modification
  modified_base 25: mod_base = OTHER; note = n appears after the nucleotide having the phosphorothioate modification
  misc_difference 27..31: note = n is 3-methoxypropyl hydrogen methylphosphonate
  modified_base 32: mod_base = OTHER; note = mA is adenosine ribonucleotide with a 2-O-methyl modification
  modified_base 33: mod_base = OTHER; note = n appears after the nucleotide having the phosphorothioate modification
  modified_base 34: mod_base = OTHER; note = mG is RNA methylated guanosine with a 2-O-methyl modification
  modified_base 35: mod_base = OTHER; note = n appears after the nucleotide having the phosphorothioate modification
  modified_base 36: mod_base = OTHER; note = mG is RNA methylated guanosine with a 2-O-methyl modification
  modified_base 37: mod_base = OTHER; note = mA is adenosine ribonucleotide with a 2-O-methyl modification
  modified_base 38: mod_base = OTHER; note = mT is RNA methylated uridine with a 2-O-methyl modification
  modified_base 39: mod_base = OTHER; note = mG is RNA methylated guanosine with a 2-O-methyl modification
  modified_base 40: mod_base = OTHER; note = n is adenosine ribonucleotide with a 2-fluoro modification
  modified_base 42: mod_base = OTHER; note = n is uridine ribonucleotide with a 2-fluoro modification
  modified_base 43: mod_base = OTHER; note = n is uridine ribonucleotide with a 2-fluoro modification
  modified_base 44: mod_base = OTHER; note = n is uridine ribonucleotide with a 2-fluoro modification
  modified_base 45: mod_base = OTHER; note = mG is RNA methylated guanosine with a 2-O-methyl modification
  modified_base 47..49: mod_base = OTHER; note = mT is RNA methylated uridine with a 2-O-methyl modification
  modified_base 51..52: mod_base = OTHER; note = mA is adenosine ribonucleotide with a 2-O-methyl modification
  modified_base 54: mod_base = OTHER; note = mT is RNA methylated uridine with a 2-O-methyl modification
  modified_base 50: mod_base = OTHER; note = mC is cytidine ribonucleotide with a 2-O-methyl modification
gngntgcatcgatgcaggngngngngnnnnnmanmgnmgmamtmgncnnnmgamtmtmtmcmamamcmt

SEQ ID NO: 7 - RNA, 28 nt, Synthetic construct
  misc_feature 1..28: note = synthetic Polynucleotide
  source 1..28: mol_type = other RNA
  modified_base 1: mod_base = OTHER; note = uracil
  modified_base 5: mod_base = OTHER; note = uracil
  modified_base 8: mod_base = OTHER; note = uracil
  modified_base 10: mod_base = OTHER; note = uracil
  modified_base 12: mod_base = OTHER; note = uracil
  modified_base 13: mod_base = OTHER; note = uracil
  modified_base 14: mod_base = OTHER; note = uracil
  modified_base 16: mod_base = OTHER; note = uracil
  modified_base 22: mod_base = OTHER; note = uracil
  modified_base 23: mod_base = OTHER; note = uracil
  modified_base 27: mod_base = OTHER; note = uracil
  modified_base 26: mod_base = OTHER; note = mG is RNA methylated guanosine with a 2-O-methyl modification
tcagtcgtatctttctgcagcttccmgt

SEQ ID NO: 8 - RNA, 27 nt, Synthetic construct
  misc_feature 1..27: note = synthetic Polynucleotide
  source 1..27: mol_type = other RNA
  modified_base 7: mod_base = OTHER; note = uracil
  modified_base 17: mod_base = OTHER; note = uracil
  modified_base 23: mod_base = OTHER; note = uracil
  modified_base 25: mod_base = OTHER; note = dA is adenosine having deoxyribose sugar
  modified_base 24: mod_base = OTHER; note = dG is guanosine having deoxyribose sugar
ggaagctgcagaaagatacgactdgda

SEQ ID NO: 9 - RNA, 31 nt, Synthetic construct
  source 1..31: mol_type = other RNA
  modified_base 1: mod_base = OTHER; note = mA is adenosine ribonucleotide with a 2-O-methyl modification
  modified_base 2: mod_base = OTHER; note = n appears after the nucleotide having the phosphorothioate modification
  modified_base 3: mod_base = OTHER; note = mG is RNA methylated guanosine with a 2-O-methyl modification
  modified_base 4: mod_base = OTHER; note = n appears after the nucleotide having the phosphorothioate modification
  modified_base 6: mod_base = OTHER; note = mA is adenosine ribonucleotide with a 2-O-methyl modification
  modified_base 7: mod_base = OTHER; note = Uracil
  modified_base 8: mod_base = OTHER; note = mG is RNA methylated guanosine with a 2-O-methyl modification
  modified_base 11..13: mod_base = OTHER; note = Uracil
  modified_base 14: mod_base = OTHER; note = mG is RNA methylated guanosine with a 2-O-methyl modification
  modified_base 16: mod_base = OTHER; note = mT is RNA methylated uridine with a 2-O-methyl modification
  modified_base 17: mod_base = OTHER; note = Uracil
  modified_base 18: mod_base = OTHER; note = mT is RNA methylated uridine with a 2-O-methyl modification
  modified_base 20: mod_base = OTHER; note = mA is adenosine ribonucleotide with a 2-O-methyl modification
  modified_base 23: mod_base = OTHER; note = Uracil
manmgngmatmgactttmgamttmtcmaact

SEQ ID NO: 10 - RNA, 34 nt, Synthetic construct
  source 1..34: mol_type = other RNA
  modified_base 2: mod_base = OTHER; note = n appears after the nucleotide having the phosphorothioate modification
  modified_base 4: mod_base = OTHER; note = n appears after the nucleotide having the phosphorothioate modification
  modified_base 5..6: mod_base = OTHER; note = Uracil
  modified_base 7: mod_base = OTHER; note = mG is RNA methylated guanosine with a 2-O-methyl modification
  modified_base 9: mod_base = OTHER; note = mA is adenosine ribonucleotide with a 2-O-methyl modification
  modified_base 11: mod_base = OTHER; note = mT is RNA methylated uridine with a 2-O-methyl modification
  modified_base 17: mod_base = OTHER; note = Uracil
  modified_base 20: mod_base = OTHER; note = Uracil
  modified_base 21: mod_base = OTHER; note = mC is cytidine ribonucleotide with a 2-O-methyl modification
  modified_base 23: mod_base = OTHER; note = mT is RNA methylated uridine with a 2-O-methyl modification
  modified_base 24: mod_base = OTHER; note = n appears after the nucleotide having the phosphorothioate modification
  modified_base 25: mod_base = OTHER; note = mT is RNA methylated uridine with a 2-O-methyl modification
  modified_base 26: mod_base = OTHER; note = n appears after the nucleotide having the phosphorothioate modification
  modified_base 27: mod_base = OTHER; note = mT is RNA methylated uridine with a 2-O-methyl modification
angnttmgamaamtcaaagtcatmccmtnmtnmt

SEQ ID NO: 11 - RNA, 38 nt, Synthetic construct
  source 1..38: mol_type = other RNA
  modified_base 1: mod_base = OTHER; note = mA is adenosine ribonucleotide with a 2-O-methyl modification
  modified_base 2: mod_base = OTHER; note = n appears after the nucleotide having the phosphorothioate modification
  modified_base 3: mod_base = OTHER; note = mG is RNA methylated guanosine with a 2-O-methyl modification
  modified_base 4: mod_base = OTHER; note = n appears after the nucleotide having the phosphorothioate modification
  modified_base 5: mod_base = OTHER; note = mG is RNA methylated guanosine with a 2-O-methyl modification
  modified_base 6: mod_base = OTHER; note = mA is adenosine ribonucleotide with a 2-O-methyl modification
  modified_base 7: mod_base = OTHER; note = mT is RNA methylated uridine with a 2-O-methyl modification
  modified_base 8: mod_base = OTHER; note = mG is RNA methylated guanosine with a 2-O-methyl modification
  modified_base 9: mod_base = OTHER; note = n is adenosine ribonucleotide with a 2-fluoro modification
  modified_base 11..13: mod_base = OTHER; note = n is uridine ribonucleotide with a 2-fluoro modification
  modified_base 14: mod_base = OTHER; note = mG is RNA methylated guanosine with a 2-O-methyl modification
  modified_base 16..18: mod_base = OTHER; note = mT is RNA methylated uridine with a 2-O-methyl modification
  modified_base 19: mod_base = OTHER; note = mC is cytidine ribonucleotide with a 2-O-methyl modification
  modified_base 20..21: mod_base = OTHER; note = mA is adenosine ribonucleotide with a 2-O-methyl modification
  modified_base 22: mod_base = OTHER; note = mC is cytidine ribonucleotide with a 2-O-methyl modification
  modified_base 23: mod_base = OTHER; note = mT is RNA methylated uridine with a 2-O-methyl modification
manmgnmgmamtmgncnnnmgamtmtmtmcmamamcmt

SEQ ID NO: 12 - RNA, 46 nt, Synthetic construct
  source 1..46: mol_type = other RNA
  modified_base 1: mod_base = OTHER; note = mA is adenosine ribonucleotide with a 2-O-methyl modification
  modified_base 2: mod_base = OTHER; note = n appears after the nucleotide having the phosphorothioate modification
  modified_base 3: mod_base = OTHER; note = fG is guanosine ribonucleotide with a 2-fluoro modification
  modified_base 4: mod_base = OTHER; note = n appears after the nucleotide having the phosphorothioate modification
  modified_base 5..6: mod_base = OTHER; note = mT is RNA methylated uridine with a 2-O-methyl modification
  modified_base 7: mod_base = OTHER; note = mG is RNA methylated guanosine with a 2-O-methyl modification
  modified_base 8: mod_base = OTHER; note = fA is adenosine ribonucleotide with a 2-fluoro modification
  modified_base 9..10: mod_base = OTHER; note = mA is adenosine ribonucleotide with a 2-O-methyl modification
  modified_base 11: mod_base = OTHER; note = mT is RNA methylated uridine with a 2-O-methyl modification
  modified_base 12: mod_base = OTHER; note = mC is cytidine ribonucleotide with a 2-O-methyl modification
  modified_base 13..15: mod_base = OTHER; note = mA is adenosine ribonucleotide with a 2-O-methyl modification
  modified_base 16: mod_base = OTHER; note = fG is guanosine ribonucleotide with a 2-fluoro modification
  modified_base 17: mod_base = OTHER; note = mT is RNA methylated uridine with a 2-O-methyl modification
  modified_base 18: mod_base = OTHER; note = fC is guanosine ribonucleotide with a 2-fluoro modification
  modified_base 19: mod_base = OTHER; note = mA is adenosine ribonucleotide with a 2-O-methyl modification
  modified_base 20: mod_base = OTHER; note = mT is RNA methylated uridine with a 2-O-methyl modification
  modified_base 21..22: mod_base = OTHER; note = mC is cytidine ribonucleotide with a 2-O-methyl modification
  modified_base 23: mod_base = OTHER; note = mT is RNA methylated uridine with a 2-O-methyl modification
  modified_base 24: mod_base = OTHER; note = n appears after the nucleotide having the phosphorothioate modification
  modified_base 25: mod_base = OTHER; note = mT is RNA methylated uridine with a 2-O-methyl modification
  modified_base 26: mod_base = OTHER; note = n appears after the nucleotide having the phosphorothioate modification
  modified_base 27: mod_base = OTHER; note = mT is RNA methylated uridine with a 2-O-methyl modification
mannnmtmtmgnmamamtmcmamamanmtnmamtmcmcmtnmtnmt

SEQ ID NO: 13 - DNA, 26 bp, synthetic construct
  source 1..26: mol_type = other DNA
  modified_base 2: mod_base = OTHER; note = n appears after the nucleotide having the phosphorothioate
  modified_base 4: mod_base = OTHER; note = n appears after the nucleotide having the phosphorothioate
  modified_base 19: mod_base = OTHER; note = n appears after the nucleotide having the phosphorothioate
  modified_base 21: mod_base = OTHER; note = n appears after the nucleotide having the phosphorothioate
  modified_base 23: mod_base = OTHER; note = n appears after the nucleotide having the phosphorothioate
  modified_base 25: mod_base = OTHER; note = n appears after the nucleotide having the phosphorothioate
gngntgcatcgatgcaggngngngng

SEQ ID NO: 14 - DNA, 59 bp, synthetic construct
  source 1..59: mol_type = other DNA
  modified_base 2: mod_base = OTHER; note = n appears after the nucleotide having the phosphorothioate modification
  modified_base 4: mod_base = OTHER; note = n appears after the nucleotide having the phosphorothioate modification
  modified_base 19: mod_base = OTHER; note = n appears after the nucleotide having the phosphorothioate modification
  modified_base 23: mod_base = OTHER; note = n appears after the nucleotide having the phosphorothioate modification
  modified_base 25: mod_base = OTHER; note = n appears after the nucleotide having the phosphorothioate modification
  misc_difference 27..31: note = n is 3-methoxypropyl hydrogen methylphosphonate
  modified_base 32: mod_base = OTHER; note = uracil
  modified_base 36: mod_base = OTHER; note = Uracil
  modified_base 39: mod_base = OTHER; note = uracil
  modified_base 41: mod_base = OTHER; note = uracil
  modified_base 43..45: mod_base = OTHER; note = uracil
  modified_base 47: mod_base = OTHER; note = uracil
  modified_base 53..54: mod_base = OTHER; note = uracil
  modified_base 57: mod_base = OTHER; note = mG is RNA methylated guanosine with a 2-O-methyl modification
  modified_base 58: mod_base = OTHER; note = uracil
gngntgcatcgatgcaggngngngngnnnnntcagtcgtatctttctgcagcttccmgt

SEQ ID NO: 15 - DNA, 62 bp, synthetic construct
  source 1..62: mol_type = other DNA
  modified_base 2: mod_base = OTHER; note = n appears after the nucleotide having the phosphorothioate modification
  modified_base 4: mod_base = OTHER; note = n appears after the nucleotide having the phosphorothioate modification
  modified_base 19: mod_base = OTHER; note = n appears after the nucleotide having the phosphorothioate modification
  modified_base 21: mod_base = OTHER; note = n appears after the nucleotide having the phosphorothioate modification
  modified_base 23: mod_base = OTHER; note = n appears after the nucleotide having the phosphorothioate modification
  modified_base 25: mod_base = OTHER; note = n appears after the nucleotide having the phosphorothioate modification
  modified_base 27..31: mod_base = OTHER; note = n is 3-methoxypropyl hydrogen methylphosphonate
  modified_base 32: mod_base = OTHER; note = mA is adenosine ribonucleotide with a 2-O-methyl modification
  modified_base 33: mod_base = OTHER; note = n appears after the nucleotide having the phosphorothioate modification
  modified_base 34: mod_base = OTHER; note = RNA methylated guanosine with a 2-O-methyl modification
  modified_base 35: mod_base = OTHER; note = n is n appears after the nucleotide having the phosphorothioate modification
  modified_base 37: mod_base = OTHER; note = mA is adenosine ribonucleotide with a 2-O-methyl modification
  modified_base 38: mod_base = OTHER; note = Uracil
  modified_base 39: mod_base = OTHER; note = mG is RNA methylated guanosine with a 2-O-methyl modification
  modified_base 42..44: mod_base = OTHER; note = Uracil
  modified_base 45: mod_base = OTHER; note = mG is RNA methylated guanosine with a 2-O-methyl modification
  modified_base 47: mod_base = OTHER; note = mT is RNA methylated uridine with a 2-O-methyl modification
  modified_base 48: mod_base = OTHER; note = Uracil
  modified_base 49: mod_base = OTHER; note = mT is RNA methylated uridine with a 2-O-methyl modification
  modified_base 51: mod_base = OTHER; note = mA is adenosine ribonucleotide with a 2-O-methyl modification
  modified_base 54: mod_base = OTHER; note = Uracil
gngntgcatcgatgcaggngngngngnnnnnmanmgngmatmgactttmgamttmtcmaact

SEQ ID NO: 16 - RNA, 22 nt, synthetic construct
  misc_feature 1..22: note = synthetic Polynucleotide
  source 1..22: mol_type = other RNA
  modified_base 1: mod_base = OTHER; note = uracil
  modified_base 8: mod_base = OTHER; note = uracil
  modified_base 12: mod_base = OTHER; note = uracil
  modified_base 13: mod_base = OTHER; note = uracil
  modified_base 17: mod_base = OTHER; note = uracil
  modified_base 21: mod_base = OTHER; note = uracil
  modified_base 22: mod_base = OTHER; note = uracil
tgagaactgaattccatgggtt

SEQ ID NO: 17 - RNA, 22 nt, synthetic construct
  misc_feature 1..22: note = synthetic Polynucleotide
  source 1..22: mol_type = other RNA
  modified_base 5: mod_base = OTHER; note = uracil
  modified_base 10: mod_base = OTHER; note = uracil
  modified_base 11: mod_base = OTHER; note = uracil
  modified_base 15: mod_base = OTHER; note = uracil
  modified_base 16: mod_base = OTHER; note = uracil
  modified_base 18: mod_base = OTHER; note = uracil
cccatggaattcagttctcaaa